(19) United States
(12) Patent Application Publication
(10) Pub. No.: US 2003/0203915 A1
(43) Pub. Date: Oct. 30, 2003

(54) Nitric oxide donors, compositions and methods of use related applications

(76) Inventors: Xinqin Fang, Lexington, MA (US); David S. Garvey, Dover, MA (US); Ricky D. Gaston, Malden, MA (US); Chia-En Lin, Burlington, MA (US); Ramani R. Ranatunga, Lexington, MA (US); Stewart K. Richardson, Tolland, CT (US); Tiansheng Wang, Concord, MA (US); Weiheng Wang, Bedford, MA (US); Shiow-Jyi Wey, Woburn, MA (US)

Correspondence Address: EDWARD D GRIEFF, HALE & DORR LLP, 1455 PENNSYLVANIA AVE, NW, WASHINGTON, DC, 20004, US

(21) Appl. No.: 10/407,420

(22) Filed: Apr. 7, 2003

Related U.S. Application Data
(60) Provisional application No. 60/369,873, filed on Apr. 5, 2002.

(51) Int. Cl. A61K 31/496; A61K 31/505; A61K 31/325; A61K 31/215

(52) U.S. Cl. 514/253.01; 514/305; 514/275; 514/508; 514/484; 544/360; 544/322; 558/480

(57) ABSTRACT

The invention describes novel nitric oxide donors and novel compositions comprising at least one nitric oxide donor. The invention also provides novel compositions comprising at least one nitric oxide donor, and, optionally, at least one therapeutic agent. The compounds and compositions of the invention can also be bound to a matrix. The invention also provides methods for treating cardiovascular diseases, for the inhibition of platelet aggregation and platelet adhesion caused by the exposure of blood to a medical device, for treating pathological conditions resulting from abnormal cell proliferation; transplantation rejections, autoimmune, inflammatory, proliferative, hyperproliferative, vascular diseases; for reducing scar tissue or for inhibiting wound contraction, particularly the prophylactic and/or therapeutic treatment of restenosis by administering the nitric oxide donor optionally in combination with at least one therapeutic agent. The invention also provides methods for treating inflammation, pain, fever, gastrointestinal disorders, respiratory disorders and sexual dysfunctions. The nitric oxide donors donate, transfer or release nitric oxide, and/or elevate endogenous levels of endothelium-derived relaxing factor, and/or stimulate endogenous synthesis of nitric oxide and/or are substrates for nitric oxide synthase and are capable of releasing nitric oxide or indirectly delivering or transferring nitric oxide to targeted sites under physiological conditions. The therapeutic agent can optionally be substituted with at least one NO and/or NO₂ group (i.e., nitrosylated and/or nitrosated). The invention also provides novel compositions and kits comprising at least one nitric oxide donor and/or at least one therapeutic agent.

### RELATED APPLICATIONS

This application claims priority to U.S. application No. 60/369,873 filed Apr. 5, 2002.

### FIELD OF THE INVENTION

The invention describes novel nitric oxide donors and novel compositions comprising at least one nitric oxide donor. The invention also provides novel compositions comprising at least one nitric oxide donor, and, optionally, at least one therapeutic agent. The compounds and compositions of the invention can also be bound to a matrix. The invention also provides methods for treating cardiovascular diseases, for the inhibition of platelet aggregation and platelet adhesion caused by the exposure of blood to a medical device, for treating pathological conditions resulting from abnormal cell proliferation; transplantation rejections, autoimmune, inflammatory, proliferative, hyperproliferative, vascular diseases; for reducing scar tissue or for inhibiting wound contraction, particularly the prophylactic and/or therapeutic treatment of restenosis by administering the nitric oxide donor optionally in combination with at least one therapeutic agent. The invention also provides methods for treating inflammation, pain, fever, gastrointestinal disorders, respiratory disorders and sexual dysfunctions. The nitric oxide donors donate, transfer or release nitric oxide, and/or elevate endogenous levels of endothelium-derived relaxing factor, and/or stimulate endogenous synthesis of nitric oxide and/or are substrates for nitric oxide synthase and are capable of releasing nitric oxide or indirectly delivering or transferring nitric oxide to targeted sites under physiological conditions. The therapeutic agent can optionally be substituted with at least one NO and/or NO₂ group (i.e., nitrosylated and/or nitrosated). The invention also provides novel compositions and kits comprising at least one nitric oxide donor and/or at least one therapeutic agent.

### BACKGROUND OF THE INVENTION

Endothelium-derived relaxing factor (EDRF) is a vascular relaxing factor secreted by the endothelium and is important in the control of vascular tone, blood pressure, inhibition of platelet aggregation, inhibition of platelet adhesion, inhibition of mitogenesis, inhibition of proliferation of cultured vascular smooth muscle, inhibition of leukocyte adherence and prevention of thrombosis. EDRF has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

Removal of the endothelium is a potent stimulus for neointimal proliferation, a common mechanism underlying the restenosis of atherosclerotic vessels after balloon angioplasty (Liu et al., Circulation, 79:1374-1387 (1989); Fems et al., Science, 253:1129-1132 (1991)). Balloon arterial injury results in endothelial denudation and subsequent regrowth of dysfunctional endothelium (Saville, Analyst, 83:670-672 (1958)) that may contribute to the local smooth muscle cell proliferation and extracellular matrix production that result in reocclusion of the arterial lumen. Nitric oxide dilates blood vessels (Vallance et al., Lancet, 2:997-1000 (1989)), inhibits platelet activation and adhesion (Radomski et al., Br. J Pharmacol, 92:181-187 (1987)), and nitric oxide limits the proliferation of vascular smooth muscle cells in vitro (Garg et al., J. Clin. Invest., 83:1774-1777 (1986)). Similarly, in animal models, suppression of platelet-derived mitogens decreases intimal proliferation (Fems et al., Science, 253:1129-1132 (1991)). The potential importance of endothelium-derived nitric oxide in the control of arterial remodeling after injury is further supported by recent preliminary reports in humans suggesting that systemic nitric oxide donors reduce angiographic restenosis six months after balloon angioplasty (The ACCORD Study Investigators, J. Am. Coil Cardiol. 23:59A. (Abstr.) (1994)).

Another aspect of restenosis may simply be mechanical, e.g., caused by the elastic rebound of the arterial wall and/or by dissections in the vessel wall caused by the angioplasty procedure. These mechanical problems have been successfully addressed by the use of stents to tack-up dissections and prevent elastic rebound of the vessel thereby reducing the level of re-occlusion for many patients. The stent is typically inserted by catheter into a vascular lumen and expanded into contact with the diseased portion of the arterial wall, thereby providing internal support for the lumen. No material has, however, been developed that matches the blood-compatible surface of the endothelium. In fact, in the presence of blood and plasma proteins, artificial surfaces are an ideal setting for platelet deposition (Salzman et al, Phil. Trans. R. Soc. Lond., B294:389-398 (1981)). Exposure of blood to an artificial surface initiates reactions that lead to clotting or platelet adhesion and aggregation. Within seconds of blood contact, the artificial surface becomes coated with a layer of plasma proteins which serves as a new surface to which platelets readily adhere, become activated, and greatly accelerate thrombus formation (Forbes et al, Brit. Med. Bull., 34(2):201-207 (1978)).

Despite considerable efforts to develop nonthrombogenic materials, no synthetic material has been created that is free from this effect. In addition, the use of anticoagulant and platelet inhibition agents has been less than satisfactory in preventing adverse consequences resulting from the interaction between blood and artificial surfaces. Consequently, a significant need exists for the development of additional methods for inhibiting platelet deposition and thrombus formation on artificial surfaces.

There is a need in the art for effective methods for treating cardiovascular diseases and disorders, particularly, restenosis and atherosclerosis. The invention is directed to these, as well as other, important ends.

### SUMMARY OF THE INVENTION

The invention describes novel nitric oxide donors and methods for treating cardiovascular diseases and disorders by administering one or more nitric oxide donors that are capable of releasing a therapeutically effective amount of nitric oxide to a targeted site affected by a cardiovascular disease or disorder. Preferably, the methods of the invention are used for treating restenosis and atherosclerosis.

One embodiment of the invention provides novel nitric oxide donors. The nitric oxide donors are compounds that are nitrosated and/or nitrosylated through one or more sites such as oxygen (hydroxyl condensation), sulfur (sulfhydryl condensation) and/or nitrogen. The nitric oxide donors donate, transfer or release nitrogen monoxide as a charged species, i.e., nitrosonium (NO⁺) or nitroxyl (NO−), or as the neutral species, nitric oxide (NO•), and/or stimulate endogenous production of nitric oxide or EDRF in vivo and/or is a substrate for nitric oxide synthase. The invention also provides compositions comprising a therapeutically effective amount of such compounds in a pharmaceutically acceptable carrier.

Another embodiment of the invention provides compositions comprising a therapeutically effective amount of at least one nitric oxide donor, and, optionally, at least one therapeutic agent that is optionally substituted with at least one NO and/or NO₂ group (i.e., nitrosylated and/or nitrosated). The nitric oxide donor can donate, transfer or release nitrogen monoxide as a charged species, i.e., nitrosonium (NO⁺) or nitroxyl (NO−), or as the neutral species, nitric oxide (NO•), and/or stimulate endogenous production of nitric oxide or EDRF in vivo and/or is a substrate for nitric oxide synthase. The invention also provides for such compositions in a pharmaceutically acceptable carrier.

Yet another embodiment of the invention describes compositions and methods for making compositions comprising at least nitric oxide donor, and, optionally at least one therapeutic agent, that is optionally substituted with at least one NO and/or NO₂ group (i.e., nitrosylated and/or nitrosated), that are bound to a natural or synthetic matrix, which can be applied with specificity to a biological site of interest. For example, the matrix containing the nitric oxide donor can be used to coat the surface of a medical device that comes into contact with blood (including blood components, blood products and the like), vascular or non-vascular tissue.

Yet another embodiment of the invention provides methods for treating cardiovascular diseases and disorders, by administering to a patient in need thereof a therapeutically effective amount of at least one nitric oxide donor that donates, transfers or releases nitric oxide as a charged species, i.e., nitrosonium (NO⁺) or nitroxyl (NO−), or as the neutral species, nitric oxide (NO•), and/or stimulates endogenous production of nitric oxide or EDRF in vivo and/or is a substrate for nitric oxide synthase. The methods can further comprise administering a therapeutically effective amount of at least one therapeutic agent that is optionally substituted with at least one NO and/or NO₂ group (i.e., nitrosylated and/or nitrosated). The nitric oxide donors and therapeutic agents, that are optionally nitrosated and/or nitrosylated can be administered separately or as components of the same composition in one or more pharmaceutically acceptable carriers.

Yet another embodiment of the invention describes methods for the inhibition of platelet aggregation and platelet adhesion caused by the exposure of blood to a medical device by incorporating at least one nitric oxide donor that is capable of releasing a therapeutically effective amount of nitric oxide into and/or on the portion(s) of the medical device that come into contact with blood (including blood components and blood products) vascular or non-vascular tissue. The methods can further comprise incorporating at least one therapeutic agent into and/or on the portion(s) of the medical device that come into contact with blood, vascular or non-vascular tissue. Alternatively the methods can comprise incorporating at least one therapeutic agent substituted with at least one NO and/or NO₂ group (i.e., nitrosylated and/or nitrosated).

Another embodiment of the invention relates to the local administration of at least one nitric oxide donor, and, optionally, at least one therapeutic agent optionally substituted with at least one NO and/or NO₂ group (i.e., nitrosylated and/or nitrosated), to treat injured tissue, such as damaged blood vessels.

The invention also provides methods using the compounds and compositions described herein to treat pathological conditions resulting from abnormal cell proliferation; transplantation rejections, autoimmune, inflammatory, proliferative, hyperproliferative or vascular diseases; for reducing scar tissue or for inhibiting wound contraction, by administering to a patient in need thereof a therapeutically effective amount of at least one of the compounds and/or compositions described herein. In these methods, the at least one nitric oxide donor and therapeutic agent, that is optionally nitrosated and/or nitrosylated, can be administered separately or as components of the same composition in one or more pharmaceutically acceptable carriers.

The invention also provides methods using the compounds and compositions described herein for treating and/or reducing inflammation, pain, and fever; for decreasing or reversing the gastrointestinal, renal, respiratory and other toxicities resulting from the use of drugs, such as nonsteroidal antiinflammatory compounds; for treating gastrointestinal disorders; for treating inflammatory disease states and disorders; for treating ophthalmic diseases or disorders; for treating and/or improving the gastrointestinal properties of COX-2 inhibitors; for treating disorders resulting from elevated levels of cyclooxygenase-2; for improving the cardiovascular properties of COX-2 inhibitors; for decreasing the recurrence of ulcers; for improving gastroprotective properties, anti-Helicobacter pylori properties or antacid properties of proton pump inhibitors; for treating Helicobacter pylori and viral infections; for improving gastroprotective properties of H₂ receptor antagonists; for treating inflammations and microbial infections, multiple sclerosis, and viral infections; for treating sexual dysfunctions in males and females, for enhancing sexual responses in males and females; for treating benign prostatic hyperplasia, hypertension, congestive heart failure, variant (Printzmetal) angina, glaucoma, neurodegenerative disorders, vasospastic diseases, cognitive disorders, urge incontinence, and overactive bladder; for reversing the state of anesthesia; for treating diseases induced by the increased metabolism of cyclic guanosine 3′,5′-monophosphate (cGMP) and for treating respiratory disorders.

These and other aspects of the invention are described in detail herein.

### DETAILED DESCRIPTION OF THE INVENTION

As used throughout the disclosure, the following terms, unless otherwise indicated, shall be understood to have the following meanings.

“Cardiovascular disease or disorder” refers to any cardiovascular disease or disorder known in the art, including, but not limited to, restenosis, coronary artery disease, atherosclerosis, atherogenesis, cerebrovascular disease, angina, (particularly chronic, stable angina pectoris), ischemic disease, congestive heart failure or pulmonary edema associated with acute myocardial infarction, thrombosis, high or elevated blood pressure in hypertension (especially hypertension associated with cardiovascular surgical procedures), platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neoinitimal hyperplasia following percutaneous transluminal coronary angiograph, and the like. Complications associated with the use of medical devices may occur as a result of increased platelet deposition, activation, thrombus formation or consumption of platelets and coagulation proteins. Such complications, which are within the definition of “cardiovascular disease or disorder,” include, for example, myocardial infarction, pulmonary thromboembolism, cerebral thromboembolism, thrombophlebitis, thrombocytopenia, bleeding disorders and/or any other complications which occur either directly or indirectly as a result of the foregoing disorders.

“Restenosis” is a cardiovascular disease or disorder that refers to the closure of a peripheral or coronary artery following trauma to the artery caused by an injury such as, for example, angioplasty, balloon dilation, atherectomy, laser ablation treatment or stent insertion. Restenosis can also occur following a number of invasive surgical techniques, such as, for example, transplant surgery, vein grafting, coronary artery bypass surgery, endarterectomy, heart transplantation, ballon angioplasty, atherectomy, laser ablation, endovascular stenting, and the like.

“Atherosclerosis” is a form of chronic vascular injury in which some of the normal vascular smooth muscle cells in the artery wall, which ordinarily control vascular tone regulating blood flow, change their nature and develop “cancer-like” behavior. These vascular smooth muscle cells become abnormally proliferative, secreting substances such as growth factors, tissue-degradation enzymes and other proteins, which enable them to invade and spread into the inner vessel lining, blocking blood flow and making that vessel abnormally susceptible to being completely blocked by local blood clotting, resulting in the death of the tissue served by that artery.

“Autoimmune, inflammatory, proliferative, hyperproliferative or vascular diseases” refers to any autoimmune, inflammatory, proliferative or hyperproliferative disease or disorder known in the art whether of a chronic or acute nature, including, but not limited to, rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis; inflammatory skin diseases, such as, for example, psoriasis, dermatitis, contact dermatitis, eczema and seborrhea; surgical adhesion; tuberculosis; inflammatory lung diseases, such as, asthma, pneumoconiosis, chronic obstructive pulmonary disease, emphysema, bronchitis, nasal polyps and pulmonary fibrosis; inflammatory bowel disease, such as, Crohn's disease and ulcerative colitis; graft rejections; inflammatory diseases that affect or cause obstruction of a body passageway, such as, vasculitis, Wegener's granulomatosis and Kawasaki disease; inflammation of the eye, nose or throat, such as, neovascular diseases of the eye including neovascular glaucoma, proliferative diabetic retinopathy, retrolental fibroblasia, macular degeneration, reduction of intraocular pressure, corneal neovascularization, such as, comeal infections; immunological processes, such as, graft rejection and Steven-Johnson's syndrome, alkali bums, trauma and inflammation (of any cause); fungal infections, such as, for example, infections caused by Candida, Trichophyton, Microsporum, Eepidermophyton, Cryptococcus, Aspergillus, Coccidiodes, Paracocciciodes, Histoplasma or Blastomyces spp; food related allergies, such as, for example, migraine, rhinitis and eczema; vascular diseases, such as, arotic aneurysm. A description of inflammatory diseases can also be found in WO 92/05179, WO 98/09972, WO 98/24427, WO 99/62510 and U.S. Pat. No. 5,886,026, the disclosures of each of which are incorporated herein in their entirety.

“Pathological conditions resulting from abnormal cell proliferation” refers to any abnormal cellular proliferation of malignant or non-malignant cells in various tissues and/or organs, including but not limited to, muscle, bone, conjunctive tissues, skin, brain, lungs, sexual organs, lymphatic system, renal system, mammary cells, blood cells, liver, the digestive system, pancreas, thyroid, adrenal glands and the like. These pathological conditions can also include psoriasis; solid tumors; ovarian, breast, brain, prostate, colon, esophageal, lung, stomach, kidney and/or testicular cancer; Karposi's sarcoma, cholangiocarcinoma; choriocarcinoma; neoblastoma; Wilm's tumor; Hodgkin's disease; melanomas; multiple myelomas; chronic lymphocytic leukemias, and acute or chronic granulocytic lymphomas. The treatment of “pathological conditions resulting from abnormal cell proliferation” includes, but is not limited to, reduction of tumor size, inhibition of tumor growth and/or prolongation of the survival time of tumor-bearing patients.

“Transplantation rejection” refers to the transplant of any organ or body part, including but not limited to, heart, kidney, liver, lung, bone marrow, cornea and skin transplants.

“Artificial surface” refers to any natural or synthetic material contained in a device or apparatus that is in contact with blood, vasculature or other tissues.

“Blood” includes blood products, blood components and the like.

“Platelet adhesion” refers to the contact of a platelet with a foreign surface, including any artificial surface, such as a medical device, as well as an injured vascular or non-vascular surfaces, such as collagen. Platelet adhesion does not require platelet activation. Unactivated, circulating platelets will adhere to injured vascular surfaces or artificial surfaces via binding interactions between circulating von Willdebrand factor and platelet surface glycoprotein Ib/IX.

“Platelet aggregation” refers to the binding of one or more platelets to each other. Platelet aggregation is commonly referred to in the context of generalized atherosclerosis, not with respect to platelet adhesion on vasculature damaged as a result of physical injury during a medical procedure. Platelet aggregation requires platelet activation which depends on the interaction between the ligand and its specific platelet surface receptor.

“Platelet activation” refers either to the change in conformation (shape) of a cell, expression of cell surface proteins (e.g., the IIb/IIIa receptor complex, loss of GPIb surface protein), and secretion of platelet derived factors (e.g., serotonin, growth factors).

“Passivation” refers to the coating of a surface which renders the surface non-reactive.

“Inflammatory disease or disorder” refers to reperfusion injury to an ischemic organ, myocardial infarction, inflammatory bowel disease, rheumatoid arthritis, osteoarthritis, hypertension, psoriasis, organ transplant rejection, organ preservation, a female or male sexual dysfunction, radiation-induced injury, asthma, atherosclerosis, thrombosis, platelet aggregation, restenosis, metastasis, influenza, incontinence, stroke, bum, trauma, acute pancreatitis, pyelonephritis, hepatitis, an autoimmune disease, an immunological disorder, senile dementia, insulin-dependent diabetes mellitus, disseminated intravascular coagulation, fatty embolism, Alzheimer's disease, adult or infantile respiratory disease, carcinogenesis or a hemorrhage in a neonate.

“Patient” refers to animals, preferably mammals, more preferably humans, and includes children and adults.

“Therapeutically effective amount” refers to the amount of the compound and/or composition that is effective to achieve its intended purpose.

“Medical device” refers to any intravascular or extravascular medical devices, medical instruments, foreign bodies including implants and the like. Examples of intravascular medical devices and instruments include balloons or catheter tips adapted for insertion, prosthetic heart valves, sutures, surgical staples, synthetic vessel grafts, stents (e.g. Palmaz-Schatz, Wiktor, Crown, Mutlilink, GFX stents), stent grafts, vascular or non-vascular grafts, shunts, aneurysm fillers (including GDC, Guglilmi detachable coils), intraluminal paving systems, guide wires, embolic agents (for example, polymeric particles, spheres and liquid embolics), filters (for example, vena cava filters), drug pumps, arteriovenous shunts, artificial heart valves, artificial implants, foreign bodies introduced surgically into the blood vessels or at vascular or non-vascular sites, leads, pacemakers, implantable pulse generators, implantable cardiac defibrillators, cardioverter defibrillators, defibrillators, spinal stimulators, brain stimulators, sacral nerve stimulators, chemical sensors, breast implants, interventional cardiology devices, catheters, and the like. Examples of extravascular medical devices and instruments include plastic tubing, dialysis bags or membranes whose surfaces come in contact with the blood stream of a patient. The term “medical device” also includes bandages or any external devices that can be applied directed to the skin.

“Gastrointestinal disorder” refers to any disease or disorder of the upper and lower gastrointestinal tract of a patient including, for example, inflammatory bowel disease, Crohn's disease, irritable bowel syndrome, ulcerative colitis, peptic ulcers, stress ulcers, bleeding peptic ulcers, duodenal ulcers, infectious enteritis, colitis, diverticulitis, gastric hyperacidity, dyspepsia, gastroparesis, Zollinger-Ellison syndrome, gastroesophageal reflux disease, Helicobacter Pylori associated disease, short-bowel (anastomosis) syndrome, hypersecretory states associated with systemic mastocytosis or basophilic leukemia and hyperhistaminemia that result, for example, from neurosurgery, head injury, severe body trauma or burns.

“Upper gastrointestinal tract” refers to the esophagus, the stomach, the duodenum and the jejunum.

“Ulcers” refers to lesions of the upper gastrointestinal tract lining that are characterized by loss of tissue. Such ulcers include gastric ulcers, duodenal ulcers and gastritis.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) inhibitor” refers to a compound that selectively inhibits the cyclooxygenase-2 enzyme over the cyclooxygenase-1 enzyme. Preferably, the compound has a cyclooxygenase-2 IC₅₀ of less than about 0.5 μM, and also has a selectivity ratio of cyclooxygenase-2 inhibition over cyclooxygenase-1 inhibition of at least 50, and more preferably of at least 100. Even more preferably, the compound has a cyclooxygenase-1 IC₅₀ of greater than about 1 μM, and more preferably of greater than 20 μM. The compound can also inhibit the enzyme, lipoxygenase and/or phosphodiestase. Such preferred selectivity may indicate an ability to reduce the incidence of common NSAID-induced side effects.

“Therapeutic agent” includes any therapeutic agent that can biologically stent a vessel and/or reduce or inhibit vascular or non-vascular remodeling and/or inhibit or reduce vascular or non-vascular smooth muscle proliferation following a procedural vascular trauma. Therapeutic agent includes the pro-drugs and pharmaceutical derivatives thereof including but not limited to the corresponding nitrosated and/or nitrosylated derivatives. Although nitric oxide donors have therapeutic activity, the term “therapeutic agent” does not include the nitric oxide donors described herein, since nitric oxide donors are separately defined.

“H₂ receptor antagonist” refers to any compound that reversibly or irreversibly blocks the activation of any H₂ receptor.

“Proton pump inhibitor” refers to any compound that reversibly or irreversibly blocks gastric acid secretion by inhibiting the H⁺/K⁺-ATP ase enzyme system at the secretory surface of the gastric parietal cell.

“Viral infection” refers to both RNA and DNA viral infections. The RNA viral infections include, but are not limited to, orthomyxoviridae, paramyxoviridae, picornaviridae, rhabdoviridae, coronavaridae, togaviridae, bunyaviridae, arenaviridae and reteroviridae. The DNA viral infections include, but are not limited to, adenoviridae, proxviridae, papovaviridae, herpetoviridae and herpesviridae. The most preferable viral infections are those of the herpetoviridae family, such as, for example, herpes simplex viruses HSV-1 and HSV-2, cytomegalovirus (CMV), herpes varicella-zoster (VZV), Epstein-Barr (EBV), HHV6, HHV7, pseudorabies and rhinotracheitis, and the like.

“Vasoactive agent” refers to any therapeutic agent capable of relaxing vascular and/or nonvascular smooth muscle. Suitable vasoactive agents include, but are not limited to, potassium channel activators, calcium channel blockers, β-blockers, long and short acting α-adrenergic receptor antagonists, prostaglandins, phosphodiesterase inhibitors, adenosine, ergot alkaloids, vasoactive intestinal peptides, dopamine agonists, opioid antagonists, endothelin antagonists, thromboxane inhibitors and the like.

“Phosphodiesterase inhibitor” or “PDE inhibitor” refers to any compound that inhibits the enzyme phosphodiesterase. The term refers to selective or non-selective inhibitors of cyclic guanosine 3′,5′-monophosphate phosphodiesterases (cGMP-PDE) and cyclic adenosine 3′,5′-monophosphate phosphodiesterases (cAMP-PDE). “α-adrenergic receptor antagonists” refers to any compound that reversibly or irreversibly blocks the activation of any α-adrenergic receptor.

“Thromboxane inhibitor” refers to any compound that reversibly or irreversibly inhibits thromboxane synthesis, and includes compounds which are the so-called thromboxane A₂ receptor antagonists, thromboxane A₂ antagonists, thromboxane A₂/prostaglandin endoperoxide antagonists, thromboxane receptor (TP) antagonists, thromboxane antagonists, thromboxane synthase inhibitors, and dual acting thromboxane synthase inhibitors and thromboxane receptor antagonists.

“Thromboxane A₂ receptor antagonist” refers to any compound that reversibly or irreversibly blocks the activation of any thromboxane A₂ receptor.

“Thromboxane synthase inhibitor” refers to any compound that reversibly or irreversibly inhibits the enzyme thromboxane synthesis thereby reducing the formation of thromboxane A₂.

“Dual acting thromboxane receptor antagonist and thromboxane synthase inhibitor” refers to any compound that simultaneously acts as a thromboxane A₂ receptor antagonist and a thromboxane synthase inhibitor.

“Taxane” refers to any compound that contains the carbon core framework represented by Formula A:

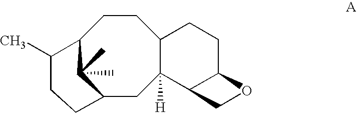

“Sexual dysfunction” refers to any sexual dysfunction in a patient, including, for example, sexual desire disorders, sexual arousal disorders, orgasmic disorders and sexual pain disorders.

“Female sexual dysfunction” refers to any female sexual dysfunction including, for example, sexual desire disorders, sexual arousal dysfunctions, orgasmic dysfunctions, sexual pain disorders, dyspareunia, and vaginismus. The female can be pre-menopausal or menopausal.

“Male sexual dysfunction” refers to any male sexual dysfunctions including, for example, male erectile dysfunction and impotence.

“Respiratory disease or disorder” refers to any pulmonary dysfunction including, for example, acute pulmonary vasoconstriction, pneumonia, traumatic injury, aspiration or inhalation injury, fat embolism in the lung, acidosis, inflammation of the lung, adult respiratory distress syndrome, acute pulmonary edema, acute mountain sickness, asthma, post cardiac surgery acute pulmonary hypertension, persistent pulmonary hypertension of the newborn, perinatal aspiration syndrome, hyaline membrane disease, acute pulmonary thromboembolism, heparin-protamine reactions, sepsis, asthma, status asthmaticus, or hypoxia (including that which may occur during one-lung anesthesia), chronic pulmonary vasoconstriction, chronic pulmonary hypertension, bronchopulmonary dysplasia, chronic pulmonary thromboembolism, idiopathic or primary pulmonary hypertension, or chronic hypoxia.

“Prodrug” refers to a compound that is made more active in vivo.

“Nitric oxide adduct” or “NO adduct” refers to compounds and functional groups which, under physiological conditions, can donate, release and/or directly or indirectly transfer any of the three redox forms of nitrogen monoxide (NO⁺, NO−, NO•), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO−, NO•), such that the biological activity of the nitrogen monoxide species is expressed at the intended site of action.

“Nitric oxide donor” or “NO donor” refers to compounds of the invention of Formulas (I) and (II) that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo, and/or are substrates for nitric oxide synthase.

“Alkyl” refers to a lower alkyl group, a haloalkyl group, a hydroxyalkyl group, an alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimetyl-butyn-1-yl, and the like.

“Bridged cycloalkyl” refers to two or more saturated or unsaturated cycloalkyl groups, saturated or unsaturated heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabycyclo(2.2.1)heptyl, 8-azabicyclo(3,2,1)oct-2-enyl, bicyclo(2.2.1)hept-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur maybe in the thio, sulfinyl or sulfonyl oxidation state. The heterocyclic ring or group can be fused to an aromatic hydrocarbon group. Heterocyclic groups can be unsubstituted or substituted with one, two, three or four substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamido and nitro. Exemplary heterocyclic groups include pyrrolyl, furyl, thienyl, 3-pyrrolinyl,4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrhydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonarnido and nitro. Exemplary substituted aryl groups include tetrafluorophenyl, pentafluorophenyl, sulfonamide, alkylsulfonyl, arylsulfonyl, and the like.

“Cycloalkenyl” refers to an unsaturated cyclic C₂-C₁₀ hydrocarbon (preferably a C₂-C₈ hydrocarbon, more preferably a C₂-C₆ hydrocarbon) which can comprise one or more carbon-carbon triple bonds.

“Arylalkyl” refers to an aryl radical, as defined herein, attached to an alkyl radical, as defined herein. Exemplary arylalkyl groups include benzyl, phenylethyl, 4-hydroxybenzyl, 3-fluorobenzyl, 2-fluorophenylethyl, and the like.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkenyl” refers to an aryl radical, as defined herein, attached to an alkenyl radical, as defined herein. Exemplary arylalkenyl groups include styryl, propenylphenyl, and the like.

“Cycloalkylalkyl” refers to a cycloalkyl radical, as defined herein, attached to an alkyl radical, as defined herein.

“Cycloalkylalkoxy” refers to a cycloalkyl radical, as defined herein, attached to an alkoxy radical, as defined herein.

“Cycloalkylalkylthio” refers to a cycloalkyl radical, as defined herein, attached to an alkylthio radical, as defined herein.

“Heterocyclicalkyl” refers to a heterocyclic ring radical, as defined herein, attached to an alkyl radical, as defined herein.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Alkoxy” refers to R₅₀O—, wherein R₅₀ is an alkyl group, as defined herein (preferably a lower alkyl group or a haloalkyl group, as defined herein). Exemplary alkoxy groups include methoxy, ethoxy, t-butoxy, cyclopentyloxy, trifluoromethoxy, and the like.

“Lower alkoxy” refers to a lower alkyl group, as defined herein, appended to an oxygen atom.

“Aryloxy” refers to R₅₅O—, wherein R₅₅ is an aryl group, as defined herein. Exemplary arylkoxy groups include napthyloxy, quinolyloxy, isoquinolizinyloxy, and the like.

“Alkylthio” refers to R₅₀S—, wherein R₅₀ is an alkyl group, as defined herein.

“Lower alkylthio” refers to a lower alkyl group, as defined herein, appended to a thio group, as defined herein.

“Arylalkoxy” or “alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Haloalkoxy” refers to an alkoxy group, as defined herein, in which one or more of the hydrogen atoms on the alkoxy group are substituted with halogens, as defined herein. Exemplary haloalkoxy groups include 1,1,1-trichloroethoxy, 2-bromobutoxy, and the like.

“Hydroxy” refers to —OH.

“Oxo” refers to ═O.

“Oxy” refers to —O⁻R₇₇⁺wherein R₇₇ is an organic or inorganic cation.

“Oxime” refers to (═N—OR₈₁) wherein R₈₁ is a hydrogen, an alkyl group, an aryl group, an alkylsulfonyl group, an arylsulfonyl group, a carboxylic ester, an alkylcarbonyl group, an arylcarbonyl group, a carboxamido group, an alkoxyalkyl group or an alkoxyaryl group as defined herein.

“Hydrazone refers to (═N—N(R₈₁))(R′₈₁))) wherein R′₈₁ is independently selected from R₈₁, and R₈₁ is as defined herein.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, and the like.

“Hydroxyalkyl” refers to a hydroxy group, as defined herein, appended to an alkyl group, as defined herein.

“Nitrate” refers to —O—NO₂.

“Nitrite” refers to —O—NO.

“Thionitrate” refers to —S—NO₂.

“Thionitrite” and “nitrosothiol” refer to SNO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Amino” refers to —NH₂, an alkylamino group, a dialkylamino group, an arylamino group, a diarylamino group, an alkylarylamino group or a heterocyclic ring, as defined herein.

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₂R₅₃N—, wherein R₅₂ and R₅₃ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino or arylalkylamino” refers to R₅₂R₅₅N—, wherein R₅₂ is an alkyl group, as defined herein, and R₅₅ is an aryl group, as defined herein.

“Alkylarylalkylamino” refers to R₅₂R₇₉N—, wherein R₅₂ is an alkyl group, as defined herein, and R₇₉ is an arylalkyl group, as defined herein.

“Alkylcycloalkylamino” refers to R₅₂R₈₀N—, wherein R₅₂ is an alkyl group, as defined herein, and R₈₀ is an cycloalkyl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an alkyl group, as defined herein. Exemplary aminoalkyl groups include dimethylaminopropyl, diphenylaminocyclopentyl, methylaminomethyl, and the like.

“Aminoaryl” refers to an aryl group to which is appended an alkylamino group, a arylamino group or an arylalkylamino group. Exemplary aminoaryl groups include anilino, N—methylanilino, N-benzylanilino, and the like.

“Thio” refers to —S—.

“Sulfinyl” refers to —S(O)—.

“Methanthial” refers to —C(S)—.

“Thial” refers to ═S.

“Sulfonyl” refers to —S(O)₂⁻.

“Sulfonic acid” refers to —S(O)₂OR₇₆, wherein R₇₆ is a hydrogen, an organic cation or an inorganic cation, as defined herein.

“Alkylsulfonic acid” refers to a sulfonic acid group, as defined herein, appended to an alkyl group, as defined herein.

“Arylsulfonic acid” refers to a sulfonic acid group, as defined herein, appended to an aryl group, as defined herein

“Sulfonic ester” refers to —S(O)₂OR₅₈, wherein R₅₈ is an alkyl group, an aryl group, or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein (preferably a lower alkyl group, as defined herein).

“Arylthio” refers to R₅₅S—, wherein R₅₅ is an aryl group, as defined herein.

“Arylalkylthio” refers to an aryl group, as defined herein, appended to an alkylthio group, as defined herein.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Alkylsulfonyloxy” refers to R₅₀—S(O)₂—O—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Arylsulfonyloxy” refers to R₅₅—S(O)₂—O—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group or an arylheterocyclic ring, as defined herein.

“Ester” refers to R₅,C(O)O— wherein R₅₁ is a hydrogen atom, an alkyl group, an aryl group or an arylheterocyclic ring, as defined herein.

“Carbamoyl” refers to —O—C(O)N(R₅₁)(R₅₇) or —N(R₅₁)C(O)OR₅₇,, wherein R₅₁ and R₅₇ are each independently a hydrogen atom, an alkyl group, an aryl group or an arylheterocyclic ring, as defined herein, or R₅₁ and R₅₇ taken together are a heterocyclic ring or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Arylalkylcarbonyl” refers to R₅₅—R₅₂—C(O)—, wherein R₅₅ is an aryl group, as defined herein, and R₅₂ is an alkyl group, as defined herein.

“Alkylarylcarbonyl” refers to R₅₂—R₅₅—C(O)—, wherein R₅₅ is an aryl group, as defined herein, and R₅₂ is an alkyl group, as defined herein.

“Heterocyclicalkylcarbonyl” refer to R₇₈C(O)— wherein R₇₈ is a heterocyclicalkyl group, as defined herein.

“Carboxylic ester” refers to —C(O)OR₅₈, wherein R₅₈ is an alkyl group, an aryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Carboxarido” refers to —C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group or an arylheterocyclic ring, as defined herein, or R₅₁ and R₅₇ taken together are a; heterocyclic ring or a-bridged cycloalkyl group, as defined herein.

Phosphoryl” refers to —P(R₇₀)(R₇₁)(R₇₂) wherein (R₇₁) and (R₇₂) are independently a lone pair of electrons, thial or oxo and and are independently a covalent bond, a hydrogen, a lower alkyl, an alkoxy, an alkylamino, a hydroxy, an oxy, an aryl or a heterocyclic ring. (R₇₁) and (R₇₂) taken together with the phosphorus to which they are attached are a heterocyclic ring.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

The invention is directed to the treatment of cardiovascular diseases and disorders in patients by administering one or more nitric oxide donors. The nitric oxide donors are compounds that are nitrosated and/or nitrosylated through one or more sites such as oxygen (hydroxyl condensation), sulfur (sulfhydryl condensation) and/or nitrogen. The nitric oxide donors donate, transfer or release nitrogen monoxide as a charged species, i.e., nitrosonium (NO⁺) or nitroxyl (NO−), or as the neutral species, nitric oxide (NO•), and/or stimulate endogenous production of nitric oxide or EDRF in vivo and/or is a substrate for nitric oxide synthase. The one or more nitric oxide donors are administered in the form of a pharmaceutical composition that further comprises a pharmaceutically acceptable carrier or diluent. The novel compounds and novel compositions of the invention are described in more detail herein.

In one embodiment, the invention describes nitric oxide donors and pharmaceutically acceptable salts thereof of Formula (I);

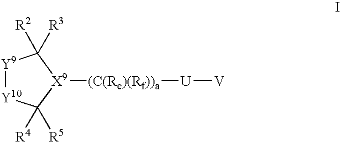

wherein:

X⁹ is CR¹⁰ or nitrogen;

Y⁹ is CR⁶R⁷NRᵢ, NR²⁵, NRᵢ—CR⁶R⁷, CR⁶R⁷—NRᵢ, CR⁶R⁷—CR⁶R⁷ or CR⁶R⁷—CR²R³;

Y¹⁰ is CR⁸R⁹ or CR⁸R⁹CR¹⁷R¹⁸;

R², R³, R⁴, R⁵, R⁶, R⁷, R⁸, R⁹, R¹⁷ and R¹⁸ are each independently a hydrogen or an alkyl group; or

R² and R³, R⁴ and R⁵, R⁶ and R⁷ or R⁸ and R⁹ each independently taken together are an oxo; or

R⁴ and R⁷ taken together with the carbon atoms to which they are attached are a cycloalkyl group; or

R⁶ and R⁹ taken together with the carbon atoms to which they are attached are a cycloalkyl group, a bridged cycloalkyl, a heterocyclic ring or an aryl group with the proviso that R⁷ and R⁸ are not present;

R⁴ and R²⁵ taken together with the carbon and nitrogen atoms to which they are attached are a heterocyclic ring;

R¹⁰ is:

(a) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— U—V;

(b) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— R₃; or

(c) —(C(Rₑ)(R_{f}))ₚ—E_{c}—(C(Rₑ)(R_{f}))ₓ—W_{d}—(C(Rₑ)(R_{f}))_{y}—Wᵢ—E;

a, c, d, g, i and j are each independently an integer from 0 to 3;

p, x, y and z are each independently an integer from 0 to 10;

W at each occurrence is independently —C(O), —C(S), —T, —(C(Rₑ)(R_{f}))ₕ, an alkyl group, an aryl group, a heterocyclic ring, an arylheterocyclic ring, —(CH₂CH₂O)_{q}, a cycloalkyl or a bridged cycloalkyl;

E at each occurrence is independently —T—, an alkyl group, an aryl group,

—(C(Rₑ)(R_{f}))ₕ, a heterocyclic ring, an arylheterocyclic ring, —(CH₂CH₂O)_{q}, a carboxylic acid, a carboxylic ester, a nitrile, an amino, a hydroxy or a phosphoryl;

h is an integer form 1 to 10;

q is an integer from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, an alkylsulfonyl, an alkylsulfonyloxy, an arylsulfonyl, arylsulphonyloxy, a sulfonic ester, a urea, a nitro, Wₕ, —U—V , or —(C(Rₑ)(R_{f}))ₖ—U—V, a phosphoryl; or Rₑ and R_{f} taken together with the carbon atom to which they are attached form a heterocyclic ring, a cycloalkyl group or a bridged cycloalkyl group; or Rₑ and R_{f} taken together are an oxo or a thial;

k is an integer from 1 to 2;

T at each occurrence is independently a covalent bond, a carbonyl, an oxygen, —S(O)ₒ or —N(Rₐ)Rᵢ;

o is an integer from 0 to 2;

U is an oxygen atom, a sulfur atom or —N(Rₐ)(Rᵢ)—;

V is —NO or —NO₂;

Rₐ is a lone pair of electrons, a hydrogen, an alkyl group or an arylalkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an amino alkyl, an amino aryl, —CH₂—C(T-Q)(Rₑ)(R_{f}), a bond to an adjacent atom creating a double bond to that atom, —(N₂O₂—)⁻•M⁺, wherein M⁺is an organic or inorganic cation;

In cases where Rₑ and R_{f} are a heterocyclic ring or Rₑ and R_{f} taken together with the carbon atoms to which they are attached are a heterocyclic ring, then Ri can be a substituent on any disubstituted nitrogen contained within the radical where Rᵢ is as defined herein.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and (C(Rₑ)(R_{f}))₂ denotes —C(Rₑ)(R_{f})—C(Rₑ)(R_{f})—.

Another embodiment of the invention describes nitric oxide donors of Formula (II):

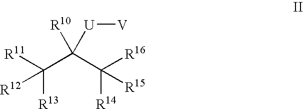

wherein:

R¹¹, R¹², R¹³, R¹⁴, R¹⁵, and R¹⁶ are each independently a hydrogen atom or an alkyl group; or

R¹¹ and R₁₂ taken together with the carbon atom to which they are attached are a cycloalkyl group or a heterocyclic ring; or

R¹³ and R¹⁴ taken together with the carbon atoms to which they are attached are a cycloalkyl group or a heterocyclic ring; or

R¹⁴ and R¹⁵ taken together with the carbon atom to which they are attached are a cycloalkyl group or a heterocyclic ring; or

R¹¹, R¹² and R¹³ taken together with the carbon atom to which they are attached are a bridged cycloalkyl group; or

R¹⁴, R¹⁵ and R¹⁶ taken together with the carbon atom to which they are attached are a bridged cycloalkyl group; or

R¹¹, R¹², R¹³, R¹⁴, R¹⁵, and R¹⁶ taken together with the carbon atoms to which they are attached are a bridged cycloalkyl group; and

R¹⁰, U, and V are as defined herein; and

with the proviso that the compounds of Formulas (I) and (II) do not include 4-aza-4-(2-methyl-2-(nitrosothio)propyl)tricyclo(5.2.1.0<2,6>)dec-8-ene-3,5-dione and the compounds of ACS registry numbers 15459-95-7; 291518-72-4; 159982-34-0; 364590-42-1; 364056-36-0; 364590-41-0; 159982-39-5; 260268-00-6; 364056-69-9; 364057-09-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1, 251369-33-2; and

with the further proviso that the compounds of Formulas (I) and (II) do not contain the following fragments as part of their structure:

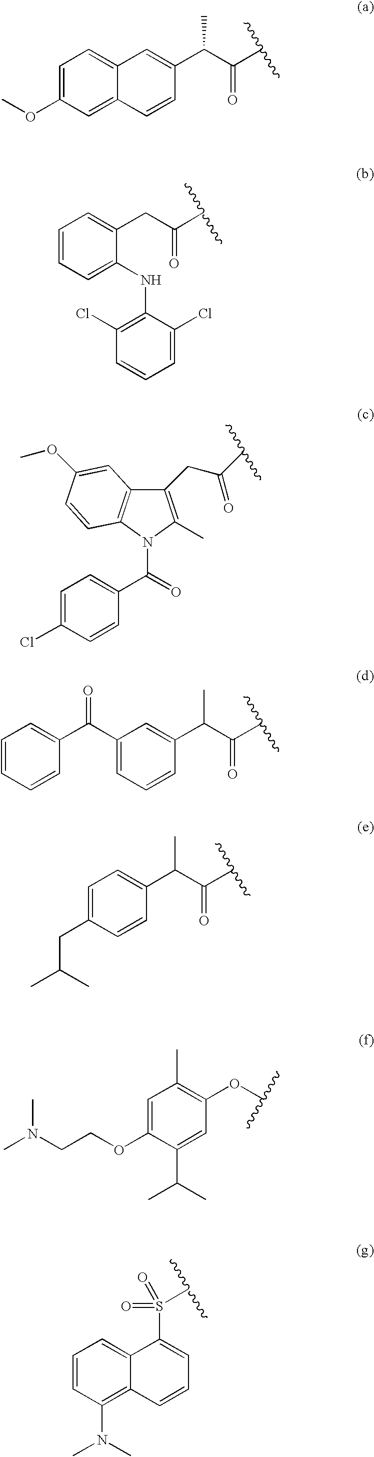

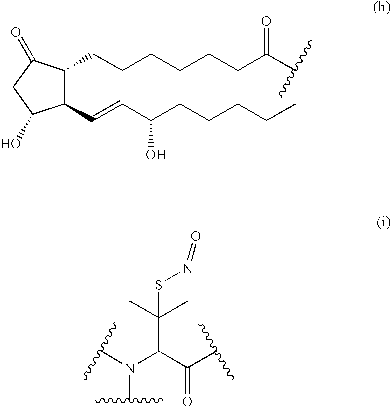

Although the compounds of Formulas (I) and (II) do not include include 4-aza-4-(2-methyl-2-(nitrosothio)propyl)tricyclo(5.2.1.0<2,6>)dec-8-ene-3,5-dione and the compounds of ACS registry numbers 15459-95-7; 291518-72-4; 159982-34-0; 364590-42-1; 364056-36-0; 364590-41-0; 159982-39-5; 260268-00-6; 364056-69-9; 364057-09-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1 and 251369-33-2; the compositions and methods described herein are intended to include compositions and methods that include 4-aza-4-(2-methyl-2-(nitrosothio)propyl)tricyclo(5.2.1.0<2,6>)dec-8-ene-3,5-dione and the compounds of ACS registry numbers 15459-95-7; 291518-72-4; 159982-34-0; 364590-42-1; 364056-36-0; 364590-41-0; 159982-39-5; 260268-00-6; 364056-69-9; 364057-090-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1 and 251369-33-2.

Compounds of the invention which have one or more asymmetric carbon atoms can exist as the optically pure enantiomers, pure diastereomers, mixtures of enantiomers, mixtures of diastereomers, racemic mixtures of enantiomers, diastereomeric racemates or mixtures of diastereomeric racemates. It is to be understood that the invention includes within its scope all such isomers and mixtures thereof.

The preferred compounds of the invention for the compounds of Formula (I) or Formula (II) are:

nitroso(1,1,3,3-tetramethyl-2-prop-2-enylindan-2-yl)thio,

2-(1,1,3,3-tetramethyl-2-(nitrosothio)indan-2-yl)ethan-1-ol,

2-(1,1,3,3-tetramethyl-2-(nitrosothio)indan-2-yl)acetic acid,

2-(1,1,3,3-tetramethyl-2-(nitrosothio)indan-2-yl)ethanenitrile,

2-((N-(2-tethyl-2-(nitrosothio)propyl)carbamoyl)methylthio)acetic acid,

nitrosothio(1,1,3,3-trimethyl-2-prop-2-enylbicyclo(2.2.1)hept-2-yl,

2-(1,3,3-trimethyl-2-(nitrosothio)bicyclo(2.2.1)hept-2-yl)ethan-1-ol,

2-(1,3,3-trimethyl-2-(nitrosothio)bicyclo(2.2.1)hept-2-yl)ethanenitrile,

(4-methoxyphenyl)-N-(2-(1,3,3-trimethyl-2-(nitrosothio)bicyclo(2.2.1)hept-2-yl) ethyl)carboxamide,

nitrosothio(1,7,7-trimethyl-2-prop-2-enylbicyclo(2.2.1)hept-2-yl,

2-(2-(nitrosothio)adamantan-2-yl)acetamide,

(1,1-bis(tert-butyl)but-3-enyl)nitrosothio,

4-(tert-butyl)-5,5-dimethyl-4-(nitrosothio)hexan-1-ol,

3-(tert-butyl)-4,4-dimethyl-3-(nitrosothio)pentanenitrile,

(1,1-diadamantanylbut-3-enyl)nitrosothio,

3-(N-(2-methyl-2-(nitrosothio)propyl)carbamoyl)pyrazine-2-carboxylic acid,

(2-methyl-2-(nitrosothio)propyl)(2-methylthiopyrimidin-4-yl)amine,

4-(N-(2-methyl-2-(nitrosothio)propyl)carbamoyl)butanoic acid,

N-(2-methyl-2-(nitrosothio)propyl)((2-methyl-2-(nitrosothio)propyl)amino) carboxamide,

1-(2-methyl-2-(nitrosothio)propyl)imidazolidine-2,4,5-trione,

3-(5-(1-methyl-1-(nitrosothio)ethyl)-3,6-dioxopiperizin-2-yl)propanoic acid,

2-(acetylamino)-N-((2-(nitrosothio)adamantan-2-yl)methyl)acetamide, adamantanylnitrosothio,

(2-methyladamantan-2-yl)nitrosothio,

phenylmethyl 4-(hydroxymethyl)-4-(nitrosothio)piperidinecarboxylate,

4-methyl-4-(N-(2-methyl-2-(nitrosothio)propyl)carbamoyl)pentanoic acid,

N,N-dimethyl-2-(2-(nitrosothio)adamantan-2-yl)acetamide,

tert-butyl 2-(2-(nitrosothio)adamantan-2-yl)acetate,

1,1-dimethyl-2-(4-(2-pyridyl)piperazinyl)ethyl)nitrosothiol,

2-(2-(nitrosothio)adamantan-2-yl)ethyl 4-methoxybenzoate,

(1,1-dimethyl-2-(2-1,2,3,4-tetrahydroisoquinolyl)ethyl)nitrosothio

4-(N-(((nitrosothiocyclohexyl)methyl)carbamoyl)butanoic acid,

N-(2-hydroxyethyl)-2-(2-(nitrosthio)adamantan-2-yl)acetamide,

N-(2-(2-(nitrosothio)adamantan-2-yl)ethyl)acetarnide,

(3-methylquinudidin-3-yl)nitrosothio hydrochloride,

2,2-bis((nitrooxy)methyl)-3-(nitrooxy)propyl 2-(2-(nitrosothio)adamantan-2-yl)acetate,

2,2-dimethyl-N-(2-methyl-2-(nitrosothio)propyl)-3-(nitrooxy)propanamide,

N-(2-methyl-2-(nitrosothio)propyl)benzamide,

2-(2-methyl-2-(nitrosothio)propyl)isoindoline-1,3-dione,

2-(N-(2-methyl-2-(nitrosothio)propyl)carbamoyl)benzoic acid,

4-(4-(2-methyl-2-(nitrosothio)propyl)piperazinyl)benzcarbonitrile,

N-(2-(dimethylbenzylammonium)ethyl)-2-(2-(nitrosothio)adamantan-2-yl)acetamide chloride,

2-(2-(nitrosothio)adamantan-2-yl)-N-(2-(trimethylammonium)ethyl)-acetamide chloride,

2(1-nitrosomercaptocyclohex-1-yl)-1,3-dioxolane,

2-(1-nitrosomercaptocyclohex-1-yl)-1,3-dioxane,

dimethyl (2,2-dicyclopropyl-2-(nitrosothio)ethyl)phosphonate,

dimethoxy ((2-(nitrosothio)adamantan-2-yl)methyl)phosphino-1-one,

((2-(ditrosothio)adaman-2-yl)methylphosphonic acid,

3-(N-(2-methyl2-(nitrosothio)propyl)carbamoyl)propanoic acid,

3-(N-(2-ethyl-2-(nitrosothio)butyl)carbamoyl)propanoic acid,

3,3-dimethyl-4-(N-(2-methyl-2-(nitrosothio)propyl)carbamoyl)butanoic acid,

3-(N-(2-methyl-2-(nitrosothio)propyl)-N-benzylcarbamoyl)propanoic acid,

2-(((N-(2-methyl-2-(nitrosothio)propyl)carbamoyl)methyl)cyclopentyl)acetic acid,

(1S,2R)-2-(N-(2-methyl-2-(nitrosothio)propyl)carbamoyl)cyclohexanecarboxylic acid,

(1R,2R)-2-(N-(2-methyl-2-(nitrosothio)propyl)carbamoyl)cyclohexanecarboxylic acid,

3-(N-(2-methyl-2-(nitrosothio)propyl)carbamoyl)-7-oxabicyclo(2.2.1)hept-5-ene-2-carboxylic acid,

3-(N-methyl-N-(2-methyl-2—(nitrosothio)propyl)carbamoyl)propanoic acid,

(tert-butoxy)-N-(2-hydroxy-1-(1-methyl-1-(nitrosothil)ethyl)ethyl)carboamide,

3-(N-(2,2-dimethylpropyl)-N-(2-methyl-2-(nitrosothio)propyl)carbamoyl)propanoic acid, or

3-(tert-butyl)-4,4-dimethyl-3-(nitrosthio)pentanenitrile.

The compounds of Formulas (I) and (II) can be synthesized following the methods described herein. The reactions are performed in solvents appropriate to the reagents, and materials used are suitable for the transformations being effected. It is understood by one skilled in the art of organic synthesis that the functionality present in the molecule must be consistent with the chemical transformation proposed. This will, on occasion, necessitate judgment by the routineer as to the order of synthetic steps, protecting groups required, and deprotection conditions. Substituents on the starting materials may be incompatible with some of the reaction conditions required in some of the methods described, but alternative methods and substituents compatible with the reaction conditions will be readily apparent to one skilled in the art. The use of sulfur and oxygen protecting groups is known in the art for protecting thiol and alcohol groups against undesirable reactions during a synthetic procedure and many such-protecting groups are known, e.g., T. H. Greene and P. G. M. Wuts, Protective Groups in Organic Synthesis, John Wiley & Sons, New York (1999), which is incorporated herein in its entirety.

The nitric oxide donors of the invention, including those described herein, which have been nitrosated and/or nitrosylated through one or more sites such as oxygen (hydroxyl condensation), sulfur (sulfhydryl condensation) and/or nitrogen. These nitrosated and/or nitrosylated compounds donate, release or transfer a biologically active form of nitrogen monoxide (nitric oxide),

Nitrogen monoxide can exist in three forms: NO− (nitroxyl), NO− (nitric oxide) and NO+ (nitrosonium). NO• is a highly reactive short-lived species that is potentially toxic to cells. This is critical because the pharmacological efficacy of NO depends upon the form in which it is delivered. In contrast to the nitric oxide radical (NO−), nitrosonium (NO⁺) does not react with O₂ or O₂− species, and functionalities capable of transferring and/or releasing NO⁺ and NO− are also resistant to decomposition in the presence of many redox metals. Consequently, administration of charged NO equivalents (positive and/or negative) does not result in the generation of toxic by-products or the elimination of the active NO moiety.

Nitric oxide donors contemplated for use in the invention are, optionally, used in combination with at least one therapeutic agent, optionally substituted with at least one NO and/or NO₂ group i.e. nitrosylated and/or nitrosated. The nitrosated and/or nitrosylated therapeutic agents can donate, release and/or directly or indirectly transfer a nitrogen monoxide species (nitric oxide), and/or stimulate the endogenous production of nitric oxide or endothelium-derived relaxing factor (EDRF) in vivo and/or elevate endogenous levels of nitric oxide or EDRF in vivo, and/or are substrates for nitric oxide synthase.

The invention is also based on the discovery that the administration of a therapeutically effective amount of the nitric oxide donor compounds and compositions described herein and/or 4-aza-4-(2-methyl-2-(nitrosothio)propyl)tricyclo(5.2.1.0<2,6>)dec-8-ene-3,5-dione are effective for treating cardiovascular diseases and disorders. For example, the patient can be administered a therapeutically effective amount of at least one nitric oxide donor of the invention. In another embodiment, the patient can be administered a therapeutically effective amount of at least one nitric oxide donor and at least one therapeutic agent, optionally substituted with at least one NO and/or NO₂ group i.e. nitrosylated and/or nitrosated. The compounds can be administered separately or in the form of a composition.

Suitable “therapeutic agents” useful in the invention, include, but are not limited to, antithrombogenic agents (such as, for example, heparin, covalent heparin, hirudin, hirulog, coumadin, protamine, argatroban, D-phenylalanyl-L-poly-L-arginyl chloromethyl ketone, and the like); thrombolytic agents (such as, for example, urokinase, streptokinase, tissueplasminogen activators, and the like); fibrinolytic agents; vasospasm inhibitors; potassium channel activators (such as, for example, nicorandil, pinacidil, cromakalim, minoxidil, aprilkalim, loprazolam and the like); calcium channel blockers (such as, for example, nifedipine, veraparmil, diltiazem, gallopamil, niludipine, nimodipins, nicardipine, and the like); antihypertensive agents (such as, for example, HYTRIN®, and the like); antimicrobial agents or antibiotics (such as, for example, adriamycin, and the like); antiplatelet agents (such as, for example, aspirin, ticlopidine, a glycoprotein IIb/IIIa inhibitor, surface glycoprotein receptors and the like); antimitotic, antiproliferative agents or microtubule inhibitors (such as, for example, taxanes, colchicine, methotrexate, azathioprine, vincristine, vinblastine, cytochalasin, fluorouracil, adriamycin, mutamycin, tubercidin, epothilone A or B, discodermolide, and the like); antisecretory agents (such as, for example, retinoid, and the like); remodelling inhibitors; antisense nucleotides (such as, for example, deoxyribonucleic acid, and the like); anti-cancer agents (such as, for example, tamoxifen citrate, acivicin, bizelesin, daunorubicin, epirubicin, mitoxantrone, and the like); steroids (such as, for example, dexamethasone, dexamethasone sodium phosphate, dexamethasone acetate, β-estradiol, and the like); non-steroidal antiinflammatory agents (NSAID); COX-2 inhibitors; 5-lipoxygenase (5-LO) inhibitors; leukotriene B₄ (LTB₄) receptor antagonists; leukotriene A4 (LTA4) hydrolase inhibitors; 5-HT agonists; HMG-CoA inhibitors; H₂ receptor antagonists; antineoplastic agents, thromboxane inhibitors; decongestants; diuretics; sedating or non-sedating anti-histamines; inducible nitric oxide synthase inhibitors; opioids, analgesics; Helicobacter pylori inhibitors; proton pump inhibitors; isoprostane inhibitors; vasoactive agents; β-agonists; anticholinergic; mast cell stabilizer; immunosuppressive agents (such as, for example cyclosporin, rapamycin, everolimus, actinomycin D and the like); growth factor antagonists or antibodies (such as, for example, trapidal (a PDGF antagonist), angiopeptin (a growth hormone antagonist), angiogenin, and the like); dopamine agonists (such as, for example, apomorphine, bromocriptine, testosterone, cocaine, strychnine, and the like); radiotherapeutic agents (such as, for example, ⁶⁰ Co (5.3 year half life), ¹⁹² Ir (73.8 days), ³² p (14.3 days), ¹¹¹ In (68 hours), ⁹⁰ Y (64 hours), ^{99m} Tc (6 hours), and the like); heavy metals functioning as radiopaque agents (such as, for example, iodine-containing compounds, barium-containing compounds, gold, tantalum, platinum, tungsten, and the like); biologic agents (such as, for example, peptides, proteins, enzymes, extracellular matrix components, cellular components, and the like); angiotensin converting enzyme (ACE) inhibitors; angiotensin II receptor antagonists; renin inhibitiors; free radical scavengers, iron chelators or antioxidants (such as, for example, ascorbic acid, alpha tocopherol, superoxide dismutase, deferoxamine, 21-aminosteroid, and the like); sex hormones (such as, for example, estrogen, and the like); antipolymerases (such as, for example, AZT, and the like); antiviral agents (such as, for example, acyclovir, famciclovir, rimantadine hydrochloride, ganciclovir sodium, Norvir®, Crixivan®, and the like); photodynamic therapy agents (such as, for example, 5-aminolevulinic acid, meta-tetrahydroxyphenylchlorin, hexadecafluoro zinc phthalocyanine, tetramethyl hematoporphyrin, rhodamine 123, and the like); antibody targeted therapy agents (such as, for example, IgG2 Kappa antibodies against Pseudomonas aeruginosa exotoxin A and reactive with A431 epidermoid carcinoma cells, monoclonal antibody against the noradrenergic enzyme dopamnine beta-hydroxylase conjugated to saporin, and the like); and gene therapy agent. Preferred therapeutic agents, include antiproliferative agents, such as, for example, taxanes; steroids such as, for example, dexamethasone, β-estradiol, immunosuppressive agents, such as for example, rapamycin, everolimus, actinomycin D, NSAIDs, such as, for example, acetaminophen, aspirin, diclofenac, ibuprofen, ketoprofen, naproxen and the like. The therapeutic agent can optionally be substituted with at least one NO and/or NO₂ group (i.e., nitrosylated and/or nitrosated). The nitric oxide donors and/or therapeutic agents can be administered separately or in the form of a composition. The nitric oxide donors, and therapeutic agents, that is optionally nitrosated and/or nitrosylated can be administered separately or in the form of a composition in one or more pharmaceutically acceptable carriers. The compounds and compositions of the invention can also be administered in combination with other medications used for the treatment of these diseases or disorders.

Suitable taxanes, include, but are not limited to, for example, paclitaxel and docetaxel, water soluble compositions of paclitaxel and docetaxel, pro-drugs of paclitaxel and docetaxel, as well as functional analogs, equivalents or derivatives of taxanes, and the like. For example, derivatives and analogs of taxanes include, but are not limited to, baccatin III, 10-deacetyltaxol, 7-xylosyl-10-deacetyltaxol, cephalomannine, 10-deacetyl-7-epitaxol, 7-epitaxol, 10-deacetylbaccatin III, 10-deacetylcephaolmannine and analogs or derivatives, and the like. Taxanes are disclosed in, for example, U.S. Pat. Nos. 4,960,790, 5,157,049, 5,284,864, 5,399,726, 5,550,261, 5,616,608, 5,629,433, 5,646,176, 5,688,977, 5,703,117, 5,760,072, 5,808,113, 5,912,263, 5,919,815, 5,965,739, 5,977,163, 5,981,564, 5,998,656, 6,017,935, 6,017,948, 6,028,205 and in WO 93/17121, WO 94/15599, WO 95/20582, WO 96/00724, WO 96/40091, WO 97/10234, WO 97/19938, WO 97/32578, WO 97/33552, WO 98/00419, WO 98/28288, WO 98/37765, WO 98/38862, WO 99/14209, WO 99/49901, WO 99/57105, WO 00/10988 and in EP 0 558 959 B1, EP 0 624 377 A2, EP 0 639 577 A1, the disclosures of each of which are incorporated by reference herein in their entirety. Taxanes and their nitrosating and/or nitrosylated derivatives are also disclosed in U.S. application Ser. No. 09/886,494, assigned to NitroMed Inc.; and in WO 00/61537, WO 00/61541 and WO 01/12584; the disclosure of each of which are incorporated by reference herein in its entirety.

Suitable anticoagulants include, but are not limited to, heparin, coumarin, aspirin, protamine, warfarin, dicumarol, phenprocoumon, indan-1,3-dione, acenocoumarol, ansindione, and the like. Suitable anticoagulants are described more fully in the literature, such as in Goodman and Gilman, The Pharmacological Basis of Therapeutics (9th Edition), McGraw-Hill, 1995, Pgs. 1341-1359; the Merck Index on CD-ROM, Twelfth Edition, Version 12:1, 1996; STN express file reg and file phar.

Suitable angiotensin-converting enzyme inhibitors, include, but are not limited to, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, trandolapril, and the like. Suitable angiotensin-converting enzyme inhibitors are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar.

Suitable angiotensin II receptor antagonists, include, but are not limited to, ciclosidomine, eprosartan, furosemide, irbesartan, losartan, saralasin, valsartan, and the like. Suitable angiotensin II receptor antagonists are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar.

Suitable renin inhibitors, include, but are not limited to, enalkrein, RO 42-5892, A 65317, CP 80794, ES 1005, ES 8891, SQ 34017, and the like. Suitable renin inhibitors are described more fully in the literature, such as in Goodman and Gilman, The Pharmacological Basis of Therapeutics (9th Edition), McGraw-Hill, 1995, Pgs. 733-838; and the Merck Index on CD-ROM, Twelfth Edition, Version 12:1, 1996; and on STN Express, file phar and file reg.

Another embodiment of the invention provides compositions comprising at least one nitric oxide donor, and, optionally, at least one therapeutic agent, optionally substituted with at least one NO and/or NO₂ group (i.e., nitrosylated and/or nitrosated). The nitric oxide donors that donates, transfers or releases nitric oxide and/or stimulates the endogenous production of NO or EDRF in vivo and/or is a substrate for nitric oxide synthase and/or at least one therapeutic agent, are bound to a matrix. Preferably, the nitric oxide donors of the invention are the compounds of Formulas (I) and (II). In a more preferred embodiment, 4-aza-4-(2-methyl-2-(nitrosothio)propyl)tricycle (5.2.1.0<2,6>)dec-8-ene-3,5-dione is bound to a matrix.

The nitric oxide donors and/or therapeutic agents and/or nitrosated and/or nitrosylated therapeutic agents, can be incorporated into a natural or synthetic matrix which can then be applied with specificity to a biological site of interest. Accordingly the nitric oxide donor and/or therapeutic agent and/or nitrosated and/or nitrosylated therapeutic agent is “bound to the matrix” which means that the nitric oxide donors and/or therapeutic agents and/or nitrosated and/or nitrosylated therapeutic agents, are physically and/or chemically associated with part of, incorporated with, attached to, or contained within the natural or synthetic matrix. In one embodiment, physical association or bonding can be achieved, for example, by coprecipitation of the nitric oxide donor and/or therapeutic agent and/or nitrosated and/or nitrosylated therapeutic agent, with the matrix. In another embodiment, chemical association or bonding can be achieved by, for example, covalent bonding of a nucleophillic moiety of the NO donor, and/or therapeutic agent and/or nitrosated and/or nitrosylated therapeutic agent, to the matrix, such that the nitric oxide donor is part of the matrix itself. In yet another embodiment, the nitric oxide donor, and/or therapeutic agent and/or nitrosated and/or nitrosylated therapeutic agent can be incorporated into a porous layer of the matrix or into pores included in the natural or synthetic matrix. The manner in which the nitric oxide donor and/or therapeutic agent and/or nitrosated and/or nitrosylated therapeutic agent, is associated, part of, attached to, incorporated with or contained within (i.e. “bound to”) the matrix is inconsequential to the invention and all means of association, incorporation, attachment, and bonding are contemplated herein. Incorporation of the nitric oxide donors, and/or therapeutic agents and/or nitrosated and/or nitrosylated therapeutic agents, into the matrix results in site-specific application, thereby enhancing selectivity of action for the released nitric oxides and/or therapeutic agents and/or nitrosated and/or nitrosylated therapeutic agents. Additionally, incorporation of the nitrosated and/or nitrosylated therapeutic agent into the matrix reduces the rate of release of the nitric oxide and the parent therapeutic agent (i.e. theraputic agent that is not nitrosated and/or nitrosylated). This prolongs the release of the nitric oxide and the parent therapeutic agent thereby allowing for efficient dosing to achieve a desired biological effect so that the frequency of dosing can be reduced.

Any of a wide variety of natural or synthetic polymers can be used as the matrix in the context of the invention. It is only necessary for the matrix to be biologically acceptable. Exemplary matrixes suitable for use in the invention are natural polymers, synthetic polymers, natural fibers, synthetic fibers, including, for example, polyolefins (such as polystyrene, polypropylene, polyethylene, high density polyethylene, polytetrafluorethylene, polyvinylidene diflouride and polyvinylchloride), polyethylenimine or derivatives thereof, polyethers (such as polyethylene glycol), polyesters (such as poly-L-lactic acid, poly-D, L-lactic, poly-D-lactic, polyglycolic, poly-(lactide/glycolide)), polyanhydrides, polyhydroxybutyrates, polyamides (such as nylon), polyurethanes, polyurethane copolymers (such as pellethane polymers), polyacrylates (such as polymethacrylate, poly (2-(methacryloyloxyethyl)-2′-(trimethylammonium)ethyl phosphate inner salt-co-n-dodecyl methacrylate), fluoro substituted polymers or copolymers (such as polymers containing one or more monomers of hexafluoropropylene (HFP), tetrafluoroethylene (TFE), vinylidenefluoride, 1-hydropentafluoropropylene, perfluoro(methyl vinyl ether), clhorotrifluoroethylene (CTFE), pentafluoropropene, trifluoroethylene, hexafluoroacetone, hexafluoroisobutylene, and the like), mixtures of polymers (such as polylactic acid/polylysine copolymers, polyurethane/polyester copolymers, polyurethane/polyether copolymers, nylon/polyether copolymers, such as vestamid), biopolymers (such as peptides, proteins, oligonucleotides, antibodies, peptide hormones, glycoproteins, glycogen and nucleic acids), starburst dendrimers, natural fibrous matrix (such as filter paper), synthetic fibrous matrix materials (such as three-dimensional lattice of synthetic polymers and copolymers) and the like. Exemplary polymers are described in U.S. Pat. Nos. 5,705,583, 5,770,645 and 5,994,444 and U.S. application Ser. No. 08/460,465, the disclosures of which are incorporated by reference herein in their entirety.

The physical and structural characteristics of the matrixes suitable for use in the invention are not critical, but depend on the application. It will be appreciated by one skilled in the art that where the matrix-nitric oxide donor of the invention is intended for local, relatively short term administration or similar administration they need not be biodegradable or bioresorbable. For some uses, such as postangioplasty, coronary bypass surgery or intimal hyperplasia associated with vascular or non-vascular graft implants or the like, it may be desirable for the matrix to slowly dissolve in a physiological environment or to be biodegradable or bioresorbable.

The nitric oxide donor and/or therapeutic agent and/or nitrosated and/or nitrosylated therapeutic agent bound to the matrix may be administered in a wide variety of forms or delivery means. Any delivery means should adequately protect the integrity of the nitric oxide prior to its release and should control the release of the nitric oxide at such a rate, in such an amount, and in such a location as to serve as an effective means for the treatment of cardiovascular diseases and disorders, including restenosis. Delivery means for local administration include, for example, sutures, vascular implants, stents, heart valves, drug pumps, drug delivery catheters infusion catheters, drug delivery guidewires, implantable medical devices and the like. Delivery means for systemic administration include, for example, solutions, suspensions, emulsions, capsules, powders, sachets, tablets, effervescent tablets, topical patches, lozenges, aerosols, liposomes, microparticles, microspheres, beads and the like. The matrix itself may be structurally sufficient to serve as a delivery means.

The nitric oxide donor and/or therapeutic agent and/or nitrosated and/or nitrosylated therapeutic agent, bound to the matrix can also be used to coat the surface of a medical device that comes into contact with blood (including blood components and blood products), vascular or non-vascular tissue thereby rendering the surface passive. U.S. Pat. Nos. 5,837,008, 5,665,077, 5,797,887 and 5,824,049, the disclosures of each of which are incorporated by reference herein in their entirety, describe methods for coating a surface of a medical device. Thus, for example, (i) all or a portion of the medical device may be coated with the nitric oxide donor and/or therapeutic agent and/or nitrosated and/or nitrosylated therapeutic agent, either as the coating per se or bound to a matrix, as described herein; or (ii) all or a portion of the medical device may be produced from a material which includes the nitric oxide donor and/or therapeutic agent and/or nitrosated and/or nitrosylated therapeutic agent, per se or bound to a matrix, as described herein.

It is also contemplated that artificial surfaces will vary depending on the nature of the surface, and such characteristics including contour, crystallinity, hydrophobicity, hydrophilicity, capacity for hydrogen bonding, and flexibility of the molecular backbone and polymers. Therefore, using routine methods, one of ordinary skill will be able to customize the coating technique by adjusting such parameters as the amount of adduct, length of treatment, temperature, diluents, and storage conditions, in order to provide optimal coating of each particular type of surface.

After the device or artificial material has been coated with the nitric oxide donor and/or therapeutic agent and/or nitrosated and/or nitrosylated therapeutic agent, it will be suitable for its intended use, including, for example, implantation as a heart valve, insertion as a catheter, insertion as a stent, or for cardiopulmonary oxygenation or hemodialysis.

The invention also describes methods for the administration of a therapeutically effective amount of the compounds and compositions described herein for treating cardiovascular diseases and disorders including, for example, restenosis and atherosclerosis. For example, the patient can be administered a therapeutically effective amount of at least one nitric oxide donor of the invention and/or 4-aza-4-(2-methyl-2-(nitrosothio)propyl)tricycle (5.2.1.0<2,6>)dec-8-ene-3,5-dione. In another embodiment, the patient can be administered a therapeutically effective amount of at least one nitric oxide donor and at least one therapeutic agent. In yet another embodiment, the patient can be administered a therapeutically effective amount of at least one nitric oxide donor and at least one therapeutic agent substituted with at least one NO and/or NO₂ group. In yet another embodiment, the patient can be administered a therapeutically effective amount of at least one nitric oxide donor and at least one therapeutic agent and at least one therapeutic agent substituted with at least one NO and/or NO₂ group. The compounds can be administered separately or in the form of a composition.

Another embodiment of the invention provides methods for the inhibition of platelet aggregation and platelet adhesion caused by the exposure of blood (including blood components or blood products) to a medical device by incorporating at least one nitric oxide donor of the invention and/or 4-aza-4-(2-methyl-2-(nitrosothio)propyl)tricycle (5.2.1.0<2,6>)dec-8-ene-3,5-dione and/or therapeutic agent and/or nitrosated and/or nitrosylated therapeutic agent, capable of releasing a therapeutically effective amount of nitric oxide, into and/or on the portion(s) of the medical device that come into contact with blood (including blood components or blood products), vascular or non-vascular tissue. The nitric oxide donor and/or therapeutic agent and/or nitrosated and/or nitrosylated therapeutic agent, may be directly or indirectly linked to the natural or synthetic polymeric material from which all or a portion of the device is made, as disclosed in U.S. Pat. No. 6,087,479, assigned to NitroMed, the disclosure of which is incorporated by reference herein in its entirety. Alternatively, the nitric oxide donor and/or therapeutic agent and/or nitrosated and/or nitrosylated therapeutic agent, may be incorporated into the body of the device which is formed of a biodegradable or bioresorbable material, including the matrix described herein. Thus the nitric oxide is released over a sustained period of the resorption or degradation of the body of the device.

Another embodiment of the invention provides methods to treat pathological conditions resulting from abnormal cell proliferation, transplant rejections, autoimmune, inflammatory, proliferative, hyperproliferative or vascular diseases, to reduce scar tissue and to inhibit wound contraction by administering to a patient in need thereof a therapeutically effective amount of the compounds and/or compositions described herein. For example, the patient can be administered a therapeutically effective amount of at least one nitric oxide donor of the invention and/or 4-aza-4-(2-methyl-2-(nitrosothio)propyl)tricycle (5.2.1.0<2,6>)dec-8-ene-3,5-dione. In another embodiment, the patient can be administered a therapeutically effective amount of at least one nitric oxide donor and at least one therapeutic agent. In yet another embodiment, the patient can be administered a therapeutically effective amount of at least one nitric oxide donor and at least one therapeutic agent substituted with at least one NO and/or NO₂ group. In yet another embodiment, the patient can be administered a therapeutically effective amount of at least one nitric oxide donor and at least one therapeutic agent and at least one therapeutic agent substituted with at least one NO and/or NO₂ group. The nitric oxide donors and/or therapeutic agents and/or therapeutic agent substituted with at least one NO and/or NO₂ group can be administered separately or in the form of a composition. The compounds and compositions of the invention can also be administered in combination with other medications used for the treatment of these disorders.

Another embodiment of the invention relates to local administration of the nitric oxide donor of the invention and/or 4-aza-4-(2-methyl-2-(nitrosothio)propyl)tricycle (5.2.1.0<2,6>)dec-8-ene-3,5-dione, -and/or therapeutic agent and/or nitrosated and/or nitrosylated therapeutic agent, to the site of injured or damaged tissue (e.g., damaged blood vessels) for the treatment of the injured or damaged tissue. Such damage may result from the use of a medical device in an invasive procedure. Thus, for example, in treating blocked vasculature by, for example, angioplasty, damage can result to the blood vessel. Such damage may be treated by use of the compounds and compositions described herein. In addition to repair of the damaged tissue, such treatment can also be used to alleviate and/or delay re-occlusions, for example, restenosis. The compounds and compositions can be locally delivered using any of the methods known to one skilled in the art, including but not limited to, a drug delivery catheter, an infusion catheter, a drug delivery guidewire, an implantable medical device, and the like. In one embodiment, all or most of the damaged area is coated with the nitric oxide donor and/or nitrosated and/or nitrosylated therapeutic agent, described herein per se or in a pharmaceutically acceptable carrier or excipient which serves as a coating matrix, including the matrix described herein. This coating matrix can be of a liquid, gel or semisolid consistency. The nitric oxide donor can be applied in combination with one or more therapeutic agents, such as those listed herein. The carrier or matrix can be made of or include agents which provide for metered or sustained release of the therapeutic agents.

In treating cardiovascular diseases and disorders, the nitric oxide donors of the invention and/or 4-aza-4-(2-methyl-2-(nitrosothio)propyl)tricycle (5.2.1.0<2,6>)dec-8-ene-3,5-dione, and/or therapeutic agent and/or nitrosated and/or nitrosylated therapeutic agent, can be administered directly to the damaged vascular or non-vascular surface intravenously by using an intraarterial or intravenous catheter, suitable for delivery of the compounds to the desired location. The location of damaged arterial surfaces is determined by conventional diagnostic methods, such as X-ray angiography, performed using routine and well-known methods available to one skilled in the art. In addition, administration of the nitric oxide donor and/or therapeutic agent and/or nitrosated and/or nitrosylated therapeutic agent, using an intraarterial or intravenous catheter is performed using routine methods well known to one skilled in the art. Typically, the compound or composition is delivered to the site of angioplasty through the same catheter used for the primary procedure, usually introduced to the carotid or coronary artery at the time of angioplasty balloon inflation. The nitric oxide donor and/or therapeutic agent and/or nitrosated and/or nitrosylated therapeutic agent, slowly decompose at body temperature over a prolonged period-of time releasing nitric oxide at a rate effective to treat, cardiovascular diseases and disorders including, for example, restenosis.

Another embodiment of the invention relates to the administration of nitric oxide donors of the invention and/or 4-aza-4-(2-methyl-2-(nitrosothio)propyl)tricycle (5.2.1.0<2,6>)dec-8-ene-3,5-dione, for treating and/or reducing inflammation, pain, and fever; for decreasing or reversing the gastrointestinal, renal and other toxicities resulting from the use of nonsteroidal antiinflammatory compounds; for treating gastrointestinal disorders; for treating inflammatory disease states and disorders; for treating ophthalmic diseases or disorders; for treating and/or improving the gastrointestinal properties of selective COX-2 inhibitors; for facilitating wound healing; for treating other disorders resulting from elevated levels of cyclooxygenase-2; for improving the cardiovascular profile of selective COX-2 inhibitors; for decreasing the recurrence of ulcers; for improving gastroprotective properties, anti-Helicobacter pylori properties or antacid properties of proton pump inhibitors; for treating Helicobacter pylori and viral infections; for improving gastroprotective properties of H₂ receptor antagonists; for treating inflammations and microbial infections, multiple sclerosis, and viral infections; for treating sexual dysfunctions in males and females, for enhancing sexual responses in males and females; for treating benign prostatic hyperplasia, hypertension, congestive heart failure, variant (Printzmetal) angina, glaucoma, neurodegenerative disorders, vasospastic diseases, cognitive disorders, urge incontinence, and overactive bladder; for reversing the state of anesthesia; for treating diseases induced by the increased metabolism of cyclic guanosine 3′,5′-monophosphate (cGMP) and for treating respiratory disorders. The nitric oxide donors of the invention can be optionally administered to a patient with at least one NSAID, COX-2 inhibitor, H₂ receptor antagonist, proton pump inhibitor, vasoactive agent, steroid, β-agonist, anticholinergic, mast cell stabilizer, PDE inhibitor, that is optionally substituted with at least one NO and/or NO₂ group (i.e., nitrosylated and/or nitrosated), to treat these diseases and disorders.

The methods for treating inflammation, pain and fever; decreasing and/or reversing gastrointestinal, renal, respiratory and other toxicities resulting from the use of drugs, such as nonsteroidal antiinflammatory compounds; and treating gastrointestinal disorders, for treating inflammatory disease states and disorders, for treating ophthalmic diseases or disorders; in a patient in need thereof, include those disclosed in U.S. Pat. Nos. 5,703,073, 6,043,232, 6.143.734, 6,051,588, 6,048,858, 6,057,347, 6,083,515, and 6,297,260 and in U.S. application Ser. No. 09/938,560, assigned to NitroMed Inc., the disclosure of each of which are incorporated by reference herein in their entirety. In these methods the at least one nitric oxide donor can optionally be administered with at least one NSAID that is optionally substituted with at least one NO and/or NO₂ group (i.e., nitrosylated and/or nitrosated).

Suitable NSAIDs, include, but are not limited to, acetaminophen, aspirin, diclofenac, ibuprofen, ketoprofen, naproxen and the like. Suitable NSAIDs are described more fully in the literature, such as in Goodman and Gilman, The Pharmacological Basis of Therapeutics (9th Edition), McGraw-Hill, 1995, Pgs. 617-657; and the Merck Index on CD-ROM, Twelfth Edition, Version 12:1, 1996. NSAIDs and their nitrosating and/or nitrosylated derivatives are also disclosed in U.S. Pat. Nos. 5,703,073, 6,043,232, 6.143.734, 6,051,588, 6,048,858, 6,057,347, 6,083,515, and 6,297,260 and in U.S. application Ser. No. 09/938,560, assigned to NitroMed Inc., and in U.S. Pat. Nos. 5,621,000, 5,700,947, 5,780,495, 5,861,426 and 6,040,341, and in WO 94/03421, WO 94/04484, WO 94/12463, WO 95/09831, WO 95/30641, WO 97/16405, WO 97/27749, WO 98/09948, WO 98/19672, WO 00/44705, WO 00/51988, WO 00/06585, WO 00/72838, WO 00/61541, WO 00/61537, WO 01/00563, WO 01/04082, WO 01/10814, WO 01/45703, WO 01/12548, WO 02/11707 and WO 02/30866 and in EP 0 759 899 B1 and EP 0 871 606 B1, the disclosure of each of which are incorporated by reference herein in their entirety.

The method for treating and/or improving the gastrointestinal properties of selective COX-2 inhibitors; for facilitating wound healing; for treating toxicity; and for treating COX-2 mediated disorders (i.e., disorders resulting from elevated levels of COX-2); for improving the cardiovascular profile of selective COX-2 inhibitors include those disclosed in U.S. application Ser. Nos. 09/741,816, 10/024,046, and in Provisional Application Nos. 60/277,950, 60/391,769, 60/392,044, 60/398,929, assigned to NitroMed Inc., the disclosure of each of which are incorporated by reference herein in their entirety. In these methods the nitric oxide donor of the invention and/or 4-aza-4-(2-methyl-2-(nitrosothio)propyl)tricycle (5.2.1.0<2,6>)dec-8-ene-3,5-dione, can optionally be administered with at least one COX-2 inhibitor that is optionally substituted with at least one NO and/or NO₂ group (i.e., nitrosylated and/or nitrosated.

Suitable COX-2 inhibitors include, but are not limited to, those disclosed in, for example, U.S. Pat. Nos. 5,134,142, .5,344,991, 5,380,738, 5,393,790, 5,409,944, 5,434,178, 5,436,265, 5,466,823, 5,474,995, 5,475,021, 5,486,534, 5,504,215, 5,508,426, 5,510,368, 5,510,496, 5,516,907, 5,521,207, 5,521,213, 5,536,752, 5,550,142, 5,552,422, 5,563,165, 5,580,985, 5,585,504, 5,596,008, 5,604,253, 5,604,260, 5,616,601, 5,620,999, 5,633,272, 5,639,780, 5,643,933, 5,677,318, 5,681,842, 5,686,460, 5,686,470, 5,691,374, 5,696,143, 5,698,584, 5,700,816, 5,710,140, 5,719,163, 5,733,909, 5,750,558, 5,753,688, 5,756,530, 5,756,531, 5,760,068, 5,776,967, 5,776,984, 5,783,597, 5,789,413, 5,807,873, 5,817,700, 5,824,699, 5,830,911, 5,840,746, 5,840,924, 5,849,943, 5,859,257, 5,861,419, 5,883,267, 5,905,089, 5,908,852, 5,908,858, 5,935,990, 5,945,539, 5,972,986, 5,980,905, 5,981,576, 5,985,902, 5,925,631, 5,990,148, 5,994,379, 5,994,381, 6,001,843, 6,002,014, 6,020,343, 6,025,353, 6,046,191, 6,071,936, 6,071,954, 6,077,869, 6,080,876, 6,083,969 and in WO 94/20480, WO 94/13635, WO 94/15932, WO 94/26731, WO 94/27980, WO 95/00501, WO 95/11883, WO 95/15315, WO 95/15316, WO 95/15318, WO 95/17317, WO 95/18799, WO 95/21817, WO 95/30652, WO 95/30656, WO 96/03392, WO 96/03385, WO 96/03387, WO 96/03388, WO 96/06840, WO 96/10021, WO 96/13483, WO 96/16934, WO 96/19469, WO 96/21667, WO 96/23786, WO 96/24584, WO 96/25405, WO 96/31509, WO 96/36623, WO 96/36617, WO 96/38418, WO 96/38442, WO 96/37467, WO 96/37468, WO 96/37469, WO 96/41626, WO 96/41645, WO 97/03953, WO 97/13767, WO 97/14691, WO 97/16435, WO 97/25045, WO 97/27181, WO 97/28120, WO 97/28121, WO 97/29776, WO 97/34882, WO 97/36863, WO 97/37984, WO 97/38986, WO 97/44027, WO 97/44028, WO 97/45420, WO 98/00416, WO 98/03484, WO 98/04527, WO 98/06708, WO 98/07714, WO 98/11080, WO 98/21195, WO 98/22442, WO 98/39330, WO 98/41511, WO 98/41516, WO 98/43649, WO 98/43966, WO 98/46594, WO 98/47509, WO 98/47871, WO 98/47890, WO 98/50033, WO 98/50075, WO 99/05104, WO 99/10331, WO 99/10332, WO 99/12930, WO 99/13799, WO 99/14194, WO 99/14195, WO 99/15205, WO 99/15503, WO 99/15505, WO 99/15513, WO 99/18960, WO 99/20110, WO 99/21585, WO 99/22720, WO 99/23087, WO 99/25695, WO 99/33796, WO 99/35130, WO 99/45913, WO 99/55830, WO 99/59634, WO 99/59635, WO 99/61016, WO 99/61436, WO 99/62884, WO 00/00200, WO 00/08024, WO 00/01380, WO 00/13685, WO 00/24719, WO 00/23433, WO 00/26216, WO 01/45703 and in EP 0 745 596 A1, EP 0 788 476 B1, EP 0 863 134 A1, EP 0 937 722 A1, and in co-pending U.S. application Ser. Nos. 09/741,816, 10/024046 and 10/102,865, and in co-pending application Ser. Nos. 60/387,433, 60/391,769, 60/392,044, and 60/398,929, the disclosures of each of which are incorporated by reference herein in their entirety.

The COX-2 inhibitors and their nitrosating-and/or-nitrosylated derivatives are disclosed in U.S. application Ser. Nos. 09/741,816, 10/024046, 10/102,865, 60/387,433, 60/391,769, 60/392,044, and 60/398,929, assigned to NitroMed Inc., the disclosure of each of which are incorporated by reference herein in their entirety.

The methods for improving the gastroprotective properties of H₂ receptor antagonists, increasing the rate of ulcer healing, decreasing the rate of recurrence of ulcers, treating inflammations, treating ophthalmic diseases and disorders, treating microbial infections, decreasing or reversing gastrointestinal toxicity and facilitating ulcer healing resulting from the administration of nonsteroidal antiinflammatory drugs (NSAIDs); improving the gastroprotective properties, anti-Helicobacter properties and antacid properties of H₂ receptor antagonists, treating gastrointestinal disorders, treating multiple sclerosis, treating ophthalmic diseases and disorders; and for treating viral infections, such as HIV disease, include those disclosed in U.S. application Ser. No. 09/441,891 and in WO 00/28988 assigned to NitroMed Inc.; the disclosure of which is incorporated by reference herein in its entirety. In these methods the at least one nitric oxide donor of the invention and/or 4-aza-4-(2-methyl-2-(nitrosothio)propyl)tricycle (5.2.1.0<2,6>)dec-8-ene-3,5-dione, can optionally be administered with at least one H₂ receptor antagonist that is optionally substituted with at least one NO and/or NO₂ group (i.e., nitrosylated and/or nitrosated).

Suitable H₂ receptor antagonists, include, but are not limited to, cimetidine, roxatidine, rantidine and the like. Suitable H₂ receptor antagonists are also described more fully in the literature, such as in Goodman and Gilman, The Pharmacological Basis of Therapeutics (9th Edition), McGraw-Hill, 1995, Pgs. 901-915; and the Merck Index on CD-ROM, Twelfth Edition, Version 12:1, 1996. The H₂ receptor antagonists and their nitrosating and/or nitrosylated derivatives are disclosed in U.S. application Ser. No. 09/441,891, assigned to NitroMed Inc., and in WO 99/45004, WO 99/44595, WO 00/61537, WO 00/61541 and WO 01/12584; the disclosure of each of which are incorporated by reference herein in their entirety.

The methods for treating gastrointestinal disorders, for improving the gastroprotective properties, anti-Helicobacter properties and antacid properties of proton pump inhibitors, for facilitating ulcer healing, for decreasing the rate of recurrence of ulcers, decreasing or reversing gastrointestinal toxicity resulting from the administration of nonsteroidal antiinflammatory drugs (NSAIDs) and/or selective COX-2 inhibitors, for facilitating ulcer healing resulting from the administration of NSAIDs and/or selective COX-2 inhibitors, treating infections caused by Helicobacter pylori and/or viruses, include those disclosed in WO 00/50037, WO 01/66088 and WO 02/00166, the disclosure of which is incorporated by reference herein in its entirety. In these methods the at least one nitric oxide donor of the invention and/or 4-aza-4-(2-methyl-2-(nitrosothio)propyl)tricycle (5.2.1.0<2,6>)dec-8-ene-3,5-dione, can optionally be administered with at least one proton pump inhibitor that is optionally substituted with at least one NO and/or NO₂ group (i.e., nitrosylated and/or nitrosated).

Suitable proton pump inhibitors, include, but are not limited to, omeprazole, esomeprazole, lansoprazole, rabeprazole, pantoprazole, and the like. Suitable proton pump inhibitors are described more fully in the literature, such as in Goodman and Gilman, The Pharmacological Basis of Therapeutics (9th Edition), McGraw-Hill, 1995, Pgs. 901-915; and the Merck Index on CD-ROM, Twelfth Edition, Version 12:1, 1996. Proton pump inhibitors and their nitrosating and/or nitrosylated derivatives are also disclosed in U.S. application Ser. No. 09/512,829, assigned to NitroMed Inc.; and in WO 99/45004, WO 99/44595, WO 00/61537, WO 00/61541, WO 01/12584, WO 01/66088, WO 00/61537 and WO 02/00166; the disclosure of each of which are incorporated by reference herein in their entirety.

The methods for treating sexual dysfunctions and/or enhancing sexual responses in patients, including males and females, include those disclosed in U.S. Pat. Nos. 5,932,538, 5,994,294, 5,874,437, 5,958,926 reissued as U.S. Pat. No.RE 0377234, 6,294,517, 6,323,211, 6,172,060, 6,197,778, 6,177,428, 6,172,068, 6,316,457, 6,221,881, 6,232,321, 6,197,782, 6,133,272 6,211,179, 6,331,543, 6,277,884, and in U.S. application Ser. Nos. 09/280,540, 09/306,805, 09/306,809, 09/347, 424, 09/941,691, 09/429/020, 09/516,194, 09/523,677, 09/570,727, and in PCT Application No. PCT/US01/16318, all assigned to NitroMed Inc., the disclosure of each of which are incorporated by reference herein in their entirety. In these methods the at least one nitric oxide donor of the invention can optionally be administered with at least one vasoactive agent that is optionally substituted with at least one NO and/or NO₂ group (i.e., nitrosylated and/or nitrosated).

Suitable vasoactive agents, and their nitrosating and/or nitrosylated derivatives, include, but are not limited to those disclosed in U.S. Pat. Nos. 5,932,538, 5,994,294, 5,874,437, 5,958,926 reissued as U.S. Pat. No.RE 0377234, 6,294,517, 6,323,211, 6,172,060, 6,197,778, 6,177,428, 6,172,068, 6,316,457, 6,221,881, 6,232,321, 6,197,782, 6,133,272, 6,211,179, 6,331,543, 6,277,884, and in U.S. application Ser. Nos. 09/280,540, 09/306,805, 09/306,809, 09/347, 424, 09/941,691, 09/429/020, 09/516,194, 09/523,677, 09/570,727, and in PCT/US01/16318, all assigned to NitroMed Inc., and in WO 98/58910, WO 00/61537, WO 00/61541 and WO 01/12584, the disclosure of each of which are incorporated by reference herein in their entirety.

The methods for treating diseases induced by the increased metabolism of cyclic guanosine 3′,5′-monophosphate (cGMP), such as hypertension, pulmonary hypertension, congestive heart failure, myocardial infraction, stable, unstable and variant (Prinzmetal) angina, atherosclerosis, cardiac edema, renal insufficiency, nephrotic edema, hepatic edema, stroke, asthma, bronchitis, chronic obstructive pulmonary disease (COPD), cystic fibrosis, dementia, immunodeficiency, premature labor, dysmenorrhoea, benign prostatic hyperplasis (BPH), bladder outlet obstruction, incontinence, conditions of reduced blood vessel patency, e.g., postpercutaneous transluminal coronary angioplasty (post-PTCA), peripheral vascular or non-vascular disease, allergic rhinitis, cystic fibrosis, and glucoma, and diseases characterized by disorders of gut motility, e.g., irritable bowel syndrome (IBS) include those disclosed in U.S. Pat. No. 6,331,543 and in U.S. application Ser. No. 09/387,727, assigned to NitroMed Inc., the disclosure of each of which are incorporated by reference herein in their entirety. In these methods the at least one nitric oxide donor of the invention and/or 4-aza-4-(2-methyl-2-(nitrosothio)propyl)tricycle (5.2.1.0<2,6>)dec-8-ene-3,5-dione, can optionally be administered with at least one phosphodiesterase inhibitor that is optionally substituted with at least one NO and/or NO₂ group (i.e., nitrosylated and/or nitrosated), and/or at least one, at least one nitric oxide donor.

Suitable phosphodiesterase inhibitors, include but are not limited to, filaminast, piclamilast, rolipram, Org 20241, MCI-154, roflumilast, toborinone, posicar, lixazinone, zaprinast, sildenafil, pyrazolopyrimidinones (such as those disclosed in WO 98/49166), motapizone, pimobendan, zardaverine, siguazodan, CI 930, EMD 53998, imazodan, saterinone, loprinone hydrochloride, 3-pyridinecarbonitrile derivatives, denbufyllene, albifylline, torbafylline, doxofylline, theophylline, pentoxofylline, nanterinone, cilostazol, cilostamide, MS 857, piroximone, milrinone, amrinone, tolafentrine, dipyridamole, papaverine, E4021, thienopyrimidine derivatives (such as those disclosed in WO 98/17668), triflusal, ICOS-351, tetrahydropiperazino(1,2-b)beta-carboline-1,4-dione derivatives (such as those disclosed in U.S. Pat. No. 5,859,006, WO 97/03985 and WO 97/03675), carboline derivatives, (such as those disclosed in WO 97/43287), 2-pyrazolin-5-one derivatives (such as those disclosed in U.S. Pat. No. 5,869,516), fused pyridazine derivatives (such as those disclosed in U.S. Pat. No. 5,849,741), quinazoline derivatives (such as those disclosed in U.S. Pat. No. 5,614,627), anthranilic acid derivatives (such as those disclosed in U.S. Pat. No. 5,714,993), imidazoquinazoline derivatives (such as those disclosed in WO 96/26940), and in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), and the like. Also included are those phosphodiesterase inhibitors disclosed in WO 99/21562 and WO 99/30697 and in U.S. application Ser. No. 09/387,727. Phosphodiesterase inhibitors and their nitrosated and/or nitrosylated derivatives are also disclosed in U.S. Pat. Nos. 5,874,437, 5,958,926, reissued as U.S. Pat. No. RE 0377234, 6,172,060, 6,197,778, 6,177,428, 6,172,068, 6,221,881, 6,232,321, 6,197,782, 6,133,272, 6,211,179, 6,316,457, 6,331,543, and U.S. applications Ser. Nos. 09/941,691, assigned to NitroMed Inc., and in WO 00/61537, WO 00/61541 and WO 01/12584. The disclosure of each of which are incorporated herein by reference in their entirety.

The methods for treating benign prostatic hyperplasia, hypertension, congestive heart failure, variant (Printzmetal) angina, glaucoma, neurodegenerative disorders, vasospastic diseases, cognitive disorders, urge incontinence, or overactive bladder, or to reverse the state of anesthesia include those disclosed in U.S. application Ser. No. 09/387,724, assigned to NitroMed Inc., the disclosure of which is incorporated by reference herein in its entirety. In these methods the at least one nitric oxide donor of the invention and/or 4-aza-4-(2-methyl-2-(nitrosothio)propyl)tricycle (5.2.1.0<2,6>)dec-8-ene-3,5-dione, can optionally be administered with at least one α-adrenergic receptor antagonist that is optionally substituted with at least one NO and/or NO₂ group (i.e., nitrosylated and/or nitrosated).

Suitable α-adrenergic receptor antagonist include but are not limited to those disclosed in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and The Merck Index (12th Ed.), Merck & Co., Inc. (1996), and in U.S. application Ser. No. 09/387,724, assigned to NitroMed Inc. The α-Adrenergic receptor antagonist and their nitrosating and/or nitrosylated derivatives are also disclosed in U.S. Pat. Nos. 5,932,538 and 5,994,294, 6,294,517, and in U.S. applications Ser. No. 09/387,724 assigned to NitroMed Inc., and in WO 00/61537, WO 00/61541, WO 01/12584. The disclosures of each of which are incorporated herein by reference in their entirety.

The methods for treating respiratory disorders, such as asthma, include those disclosed in U.S. Pat. No. 5,824,669, reissued as U.S. Pat. Nos. RE 037,611, 6,197,762, 6,331,543, and in U.S. application Ser. No. 09/689,851 assigned to NitroMed Inc., the disclosure of which are incorporated by reference herein in their entirety. In these methods the at least one nitric oxide donor of the invention and/or 4-aza-4-(2-methyl-2-(nitrosothio)propyl)tricycle (5.2.1.0<2,6>)dec-8-ene-3,5-dione, can optionally be administered with at least one steroid, β-agonist, anticholinergic, mast cell stabilizer or PDE inhibitor, that is optionally substituted with at least one NO and/or NO₂ group (i.e., nitrosylated and/or nitrosated), and/or at least one NO donor.

Suitable steroids, β-agonists, anticholinergics, mast cell stabilizers and PDE inhibitors and their nitrosating and/or nitrosylated derivatives include those disclosed in U.S. Pat. Nos. 5,824,669, reissued as U.S. Pat. No. RE 037,611, 5,958,926 reissued as U.S. Pat. No. RE 0377234, 6,197,762, 6,331,543, and in U.S. application Ser. Nos. 09/511,232 and 09/689,851 assigned to NitroMed Inc., and in U.S. Pat. Nos. 5,707,984, 5,792,758, 5,837,698 and 5,985,862, and in WO 97/41144, WO 97/40836, WO 97/21724, WO 97/21721, WO 98/15568, WO 00/06531, WO 00/61604 and WO 01/12584. The disclosures of each of which are incorporated herein by reference in their entirety.

When administered in vivo, the compounds and compositions of the invention can be administered in combination with pharmaceutically acceptable carriers and in dosages described herein. When the compounds and compositions of the invention are administered as a mixture of at least one nitric oxide donor and/or at least one therapeutic agent and/or at least one nitrosated and/or nitrosylated therapeutic agent, they can also be used in combination with one or more additional compounds which are known to be effective against the specific disease state targeted for treatment (e.g., therapeutic agents). The nitric oxide donors and/or therapeutic agents and/or nitrosated and/or nitrosylated therapeutic agent can be administered simultaneously with, subsequently to, or prior to administration of the other additional compounds.

The compounds and compositions of the invention can be administered by any available Land-effective delivery system including, but not limited to, orally, bucally, parenterally, by inhalation spray, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques.

Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, pastes, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

Solid dosage forms for oral administration can include capsules, tablets, effervescent tablets, chewable tablets, pills, powders, sachets, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as cocoa butter and polyethylene glycols which are solid at room temperature but liquid at bodytemperature, such that they will melt and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol and/or dextran. Optionally, the suspension may also contain stabilizers.

Solvents useful in the practice of this invention include pharmaceutically acceptable, water-miscible, non-aqueous solvents. In the context of this invention, these solvents should be taken to include solvents that are generally acceptable for pharmaceutical use, substantially water-miscible, and substantially non-aqueous. Preferably, these solvents are also non-phthalate plasticizer leaching solvents, so that, when used in medical equipment, they substantially do not leach phthalate plasticizers that may be present in the medical equipment. More preferably, the pharmaceutically-acceptable, water-miscible, non-aqueous solvents usable in the practice of this invention include, but are not limited to, N-methyl pyrrolidone (NMP); propylene glycol; ethyl acetate; dimethyl sulfoxide; dimethyl acetamide; benzyl alcohol; 2-pyrrolidone; benzyl benzoate; C₂₋₆ alkanols; 2-ethoxyethanol; alkyl esters such as 2-ethoxyethyl acetate, methyl acetate, ethyl acetate, ethylene glycol diethyl ether, or ethylene glycol dimethyl ether; (S)-(−)-ethyl lactate; acetone; glycerol; alkyl ketones such as methylethyl ketone or dimethyl sulfone; tetrahydrofuran; cyclic alkyl amides such as caprolactam; decylmethylsulfoxide; oleic acid; aromatic amines such as N,N-diethyl-m-toluamide; or 1-dodecylazacycloheptan-2-one.

The most preferred pharmaceutically-acceptable, water-miscible, non-aqueous solvents are N-methyl pyrrolidone (NMP), propylene glycol, ethyl acetate, dimethyl sulfoxide, dimethyl acetamide, benzyl alcohol, 2-pyrrolidone, or benzyl benzoate. Ethanol may also be used as a pharmaceutically-acceptable, water-miscible, non-aqueous solvent according to the invention, despite its negative impact on stability. Additionally, triacetin may also be used as a pharmaceutically-acceptable, water-miscible, non-aqueous solvent, as well as functioning as a solubilizer in certain circumstances. NMP may be available as PHARMASOLVE® from International Specialty Products (Wayne, N.J.). Benzyl alcohol may be available from J. T. Baker, Inc. Ethanol may be available from Spectrum, Inc. Triacetin may be available from Mallinkrodt, Inc.

The compositions of this invention can further include solubilizers. Solubilization is a phenomenon that enables the formation of a solution. It is related to the presence of amphiphiles, that is, those molecules that have the dual properties of being both polar and non-polar in the solution that have the ability to increase the solubility of materials that are normally insoluble or only slightly soluble, in the dispersion medium. Solubilizers often have surfactant properties. Their function may be to enhance the solubility of a solute in a solution, rather than acting as a solvent, although in exceptional circumstances, a single compound may have both solubilizing and solvent characteristics. Solubilizers useful in the practice of this invention include, but are not limited to, triacetin, polyethylene glycols (such as, for example, PEG 300, PEG 400, or their blend with 3350, and the like), polysorbates (such as, for example, Polysorbate 20, Polysorbate 40, Polysorbate 60, Polysorbate 65, Polysorbate 80, and the like), poloxamers (such as, for example, Poloxamer 124, Poloxamer 188, Poloxamer 237, Poloxamer 338, Poloxamer 407, and the like), polyoxyethylene ethers (such as, for example, Polyoxyl 2 cetyl ether, Polyoxyl 10 cetyl ether, and Polyoxyl 20 cetyl ether, Polyoxyl 4 lauryl ether, Polyoxyl 23 lauryl ether, Polyoxyl 2 oleyl ether, Polyoxyl 10 oleyl ether, Polyoxyl 20 oleyl ether, Polyoxyl 2 stearyl ether, Polyoxyl 10 stearyl ether, Polyoxyl 20 stearyl ether, Polyoxyl 100 stearyl ether, and the like), polyoxylstearates (such as, for example, Polyoxyl 30 stearate, Polyoxyl 40 stearate, Polyoxyl 50 stearate, Polyoxyl 100 stearate, and the like), polyethoxylated stearates (such as, for example, polyethoxylated 12-hydroxy stearate, and the like), and Tributyrin.

Other materials that may be added to the compositions of the invention include cyclodextrins, and cyclodextrin analogs and derivatives, and other soluble excipients that could enhance the stability of the inventive composition, maintain the product in solution, or prevent side effects associated with the administration of the inventive composition. Cyclodextrins may be available as ENCAPSIN® from Janssen Pharmaceuticals.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules, nanoparticles, and the like. The required dosage can be administered as a single unit or in a sustained release form.

The bioavailabilty of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the invention may be of any configuration suitable for sustained release. Preferred sustained release therapeutic dosage forms exhibit one or more of the following characteristics: microparticles (e.g., from about 0.5 micrometers to about 100 micrometers in diameter, preferably about 0.5 to about 2 micrometers; or from about 0.01 micrometers to about 200 micrometers in diameter, preferably from about 0.5 to about 50 micrometers, and more preferably from about 2 to about 15 micrometers) or nanoparticles (e.g., from about 1.0 nanometer to about 1000 nanometers in diameter, preferably about 50 to about 250 nanometers ; or from about 0.01 nanometer to about 1000 nanometers in diameter, preferably from about 50 to about 200 nanometers), free flowing powder structure; biodegradable structure designed to biodegrade over a period of time between from about 0.5 to about 180 days, preferably from about 1 to 3 to about 150 days, more preferably from about 3 to about 180 days, and most preferably from about 10 to about 21 days; or non-biodegradable structure to allow the therapeutic agent diffusion to occur over a time period of between from about 0.5 to about 180 days, more preferably from about 30 to about 120 days; or from about 3 to about 180 days, more preferably from about 10 to about 21 days; biocompatible with target tissue and the local physiological environment into which the dosage form to be administered, including yielding biocompatible biodegradation products; facilitate a stable and reproducible dispersion of therapeutic agent therein, preferably to form a therapeutic agent-polymer matrix, with active therapeutic agent release occurring by one or both of the following routes: (1) diffusion of the therapeutic agent through the dosage form (when the therapeutic agent is soluble in the shaped polymer or polymer mixture defining the dimensions of the dosage form); or (2) release of the therapeutic agent as the dosage form biodegrades; and/or for targeted dosage forms; capability to have, preferably, from about 1 to about 10,000 binding protein/peptide to dosage form bonds and more preferably, a maximum of about 1 binding peptide to dosage form bond per 150 square angstroms of particle surface area. The total number of binding protein/peptide to dosage form bonds depends upon the particle size used. The binding proteins or peptides are capable of coupling to the particles of the therapeutic dosage form through covalent ligand sandwich or non-covalent modalities as set forth herein.

Nanoparticle sustained release therapeutic dosage forms are preferably biodegradable and, optionally, bind to the vascular or non-vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Preferred larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Preferred sustained release dosage forms of the invention comprise biodegradable microparticles or nanoparticles. More preferably, biodegradable microparticles or nanoparticles are formed of a polymer containing matrix that biodegrades by random, nonenzymatic, hydrolytic scissioning to release therapeutic agent, thereby forming pores within the particulate structure.

The compounds and compositions of the invention can be formulated as pharmaceutically acceptable salts. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitrous (nitrite salt), nitric (nitrate salt), carbonic, sulfuric, phosphoric acid, and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesuifonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these salts may be prepared by conventional means from the corresponding compound by reacting, for example, the appropriate acid or base with the compound.

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The invention also provides pharmaceutical kits comprising one or more containers filled with one or more of the ingredients of the pharmaceutical compounds and/or compositions of the invention, including, one or more nitric oxide donors, and one or more therapeutic agents, optionally nitrosated and/or nitrosylated, described herein. Such kits can also include, for example, other compounds and/or compositions (e.g., therapeutic agents, permeation enhancers, lubricants, and the like), a device(s) for administering the compounds and/or compositions, and written instructions in a form prescribed by a governmental agency regulating the manufacture, use or sale of pharmaceuticals or biological products, which instructions can also reflects approval by the agency of manufacture, use or sale for human administration.

### EXAMPLES

The following non-limiting examples further describe and enable one of ordinary skill in the art to make and use the invention.

### EXAMPLE 1

Nitroso(1,1,3,3-tetramethyl-2-prop-2-enylindan-2-yl)thio

1a. 1,1,3,3-Tetramethylindan-2-one

This was prepared as described by Langhals, E. and Langhals, H., Tetrahedron Lett., 31: 859-862, 1990. Potassium hydroxide (212 g, 3.8 mol) was pulverised and added to anhydrous DMSO (300 mL) in an oil bath preheated to 60° C. When the internal temperature reached 50° C. a solution of methyl iodide (93 mL, 213 g, 1.5 mol) and 2-indanone (25 g, 0.19 mol) in DMSO (50 mL) was added dropwise keeping the internal temperature between 50-55° C. After completion of the addition, the solution was stirred at 50° C. for 1 hour, cooled to room temperature, poured into ice-water (1.5 L) and extracted with ether (3×500 mL). The combined organic phase was washed with water (2×), dried over sodium sulfate, filtered and evaporated. The residue was sublimed at 3 mm Hg with a bath temperature of 70° C. to give the title compound (23 g, 66%). ¹H NMR (300 MHz, CDCl₃) δ7.24-7.33 (m, 4H), 1.35 (s, 12H).

1b. 1,1,3,3-Tetramethylindan-2-one hydrazone

A mixture of the product of Example 1a (21 g, 111 mmol) and hydrazine hydrate (22.5 g, 446 mmol) in acetic acid (7 mL) and ethanol (50 mL) was refluxed overnight. The solution was cooled to room temperature and then stored at 4° C. The solid was filtered to give the title compound (12.5 g) and the filtrate diluted with ether and washed with water. The organic layer was dried over sodium sulfate, filtered and evaporated. The residue was chromatographed (ethyl acetate:hexane 1:4 then 1:1) to give additional product (6.5 g, total yield 84%). ¹H NMR (300 MHz, CDCl₃) δ7.24-7.28 (m, 2H), 7.17-7.21 (m, 2H), 5.31 (br s, 2H), 1.64 (s, 6H), 1.35 (s,6H).

1c. 1,1,3,3-Tetramethylindane-2-thione

This compound was prepared as described by A Ishii et al., Bull. Chem. Soc. Jpn., 61: 861-868, 1988. A solution of triethylamine (32 mL, 23 g, 229 mmol) in benzene was cooled over ice. When the internal temperature reached 5° C., separate solutions of sulfur monochloride (8.7 mL, 14.7 g, 109 mmol) in benzene (100 mL) and the product of Example 1b (21 g, 103 mmol) in benzene (100 mL) were added at identical rates while maintaining the temperature at less than 8° C. The resulting solution was stirred for 15 minutes over ice and then for 45 min at room temperature. The reaction mixture was filtered. The filtrate was washed with water (2×), brine and dried over sodium sulfate. The residue after filtration and evaporation was chromatographed (ethyl acetate:hexane 1:19) to give the title compound (14.5 g, 70%). ¹H NMR (300 MHz, CDCl₃) δ7.30 (s, 4H), 1.50 (s, 12H).

1d. 1,1,3,3-tetramethyl-2-prop-2-enylindane-2-thiol

A solution of the product of Example 1c (10 g, 50 mmol) in ether (100 mL) was cooled over ice. To this was added a solution of allylmagnesium bromide (147 mL of 1M solution in ether, 147 mmol) dropwise. The resultant solution was stirred over ice for 30 minutes, quenched carefully with excess 2N HCl and the organic phase was dried over sodium sulfate and filtered. The residue, after evaporation, was chromatographed (ether:hexane 1:19) to give the title compound (10 g, 83%). ¹H NMR (300 MHz, CDCl₃) δ7.34-7.39 (m, 2H), 7.22-7.28 (m, 2H), 6.23 (m, 1H), 5.17-5.31 (m, 2H), 2.79 (d, J=7.1 Hz, 2H), 1.62 (s, 6H), 1.53 (s, 6H). ¹³C NMR (75 MHz, CDCl₃) δ149.0, 135.4, 127.2, 122.2, 118.1, 68.4, 50.5, 40.9, 29.1, 28.6. Anal. Calcd for C₁₆H₂₂S: C, 78.00; H, 9.00, Found: C, 77.86; H, 8.97.

1e. Nitroso(1,1,3,3-tetramethyl-2-prop-2-enylindan-2-yl)thio

To a solution of tert-butyl nitrite (405 μL, 314 mg, 3 mmol) in dichloromethane (2 mL) was added dropwise a solution of the product of Example 1d (250 mg, 1 mmol) in dichloromethane (2 mL). The resultant solution was stirred at room temperature in the dark for 45 minutes. The volatiles evaporated and the residue chromatographed (ether:hexane 1:99) to give the title compound (150 mg, 54%). ¹H NMR (300 MHz, CDCl₃) δ7.34-7.39 (m, 2H), 7.22-7.28 (m, 2H), 5.95-6.09 (m, 1H), 5.17-5.31 (m, 2H), 3.78 (d, J=6.7 Hz, 2H), 1.76 (s, 6H), 1.49 (s, 6H). ¹³C NMR (75 MHz, CDCl₃) δ149.0, 135.1, 127.6, 122.2, 118.0, 80.7, 51.6, 37.1, 29.2, 28.3. Anal. Calcd for C₁₆H₂₁NOS: C, 69.78; H, 7.69; N, 5.09, Found: C, 69.65; H, 7.69; N, 4.82.

### EXAMPLE 2

2-(1,1,3,3-Tetramethyl-2-(nitrosothio)indan-2-yl)ethan-1-ol

2a. 1-(1,1,3,3-Tetramethyl-2-prop-2-enylindan-2-ylthio)ethan-1-one

A solution of the product of Example 1d (9 g, 36.6 mmol) in pyridine (189 mL, 185 g, 2.3 mol) was cooled over ice and treated dropwise with acetic anhydride (110 mL, 119 g, 1.17 mol) and 4-dimethylaminopyridine (0.5 g). The crude reaction mixture was stirred at room temperature for 12 hours. The volatile material evaporated and the residue chromatographed (ether:hexane 1:19) to give the title compound (8.1 g, 77%). Mp 65-67° C. ¹H NMR (300 MHz, CDCl₃) δ7.19-7.24 (m, 2H), 7.06-7.11 (m, 2H), 5.85-6.02 (m, 1H), 5.00-5.17 (m, 2H), 3.19 (d, J=6.6 Hz, 2H), 2.23 (s, 3H), 1.51 (s, 6H), 1.43 (s, 6H). ¹³C NMR (75 MHz, CDCl₃) δ196.5, 149.2, 136.5, 127.7, 122.4, 117.0, 51.7, 34.8, 31.8, 29.3, 28.4. Anal. Calcd for C₁₈H₂₄OS: C, 74.95; H, 8.39, Found: C, 74.76; H, 8.38.

2b. 2-(2-Acetylthio-1,1,3,3-tetramethylindan-2-yl)ethanal

A mixture of N-methylmorpholine N-oxide (50% in water, 31 mL, 131 mmol) and the product of Example 2a (8 g, 26 mmol) in water (100 mL) were treated with acetone to give a homogeneous solution (approx 350 mL). Osmium tetroxide (8 mL of 4% aqueous solution, 1.31 mmol) was introduced and the resulting solution was stirred at room temperature overnight. The volume was reduced by evaporation and the residue diluted with more water and then extracted with ethyl acetate followed by dichloromethane. The combined organic phases were dried over sodium sulfate, filtered and evaporated. The residue was dissolved on 240 mL of 3:1 ether:THF and cooled over ice under nitrogen. Periodic acid (9 g, 39 mmol) was added in portions over 20 min. The reaction mixture was stirred over ice for 1 hour and at room temperature for 40 min. The solid was removed by filtration through Celite and the filtrate was washed with water, brine, dried over sodium sulfate, filtered and evaporated. The residue was chromatographed (ethyl acetate: hexane 1:19) to give the title compound (2 g, 25%). ¹H NMR (300 MHz, CDCl₃) δ9.73 (t, J=2.5 Hz, 1H), 7.19-7.25 (m, 2H), 7.06-7.11 (m, 2H), 3.32 (d, J=2.5 Hz, 2H), 2.31 (s, 3H), 1.46 (s, 6H), 1.42 (s, 6H). ³C NMR (75 MHz, CDCl₃) δ202.6, 196.1, 147.5, 127.7, 122.2, 71.6, 51.4, 45.0, 31.4, 29.3, 27.6. Anal. Calcd for C₁₇H₂₂O₂S: C, 70.31; H, 7.64, Found: C, 70.02; H, 7.69. LRMS (APIMS) m/z 291 (MH⁺).

2c. 2-(1,1,3,3-Tetramethyl-2-sulfanylindan-2-yl)ethan-1-ol

A solution of Example 2b (2.07 g, 7.12 mmol) in THF (80 mL) was cooled over ice and a solution of lithium aluminum hydride (1M in THF, 14.2 mL, 14.2 mmol) was added dropwise. The ice bath was removed and the resultant solution was stirred at room temperature for 45 minutes. Sodium sulfate decahydrate was added to decompose excess reducing agent. The reaction mixture was filtered and the solid washed with dichloromethane:methanol 4:1. The filtrate was dried over sodium sulfate, filtered and the residue after evaporation chromatographed (ethyl acetate:hexane 1:4) to give the title compound (1.04 g, 58%). Mp. 85-87° C. ¹H NMR (300 MHz, CDCl₃) δ7.21-7.26 (m, 2H), 7.10-7.15 (m, 2H), 4.01 (br s, 2H), 2.15-2.20 (m, 2H), 1.87-(br s, 1H), -1.50 (s, 6H), 1.38 (s, 6H), 1.32 (s, 1H). ¹³C NMR (75 MHz, CDCl₃) δ1.48.5, 127.2, 122.2, 67.9, 60.4, 50.5, 39.0, 29.3, 28.3. LRMS (APIMS) m/z 268 (MNH₄⁺).

2d. 2-(1,1,3,3-Tetramethyl-2-(nitrosothio)indan-2-yl)ethan-1-ol

An ice cooled solution of the product of Example 2c (1.04 g, 4.15 mmol) in a mixture of dichloromethane:methanol (20 mL, 1:1) was treated dropwise with tert-butyl nitrite (2.5 mL, 19 mmol). The reaction mixture was stirred at 0° C. for 15 min then at room temperature for 30 minutes. The residue after evaporation was chromatographed (ethyl acetate:hexane 1:4) to give the title compound (1.05 g, 88%). ¹H NMR (300 MHz, CDCl₃) δ7.21-7.27 (m, 2H), 7.10-7.15 (m, 2H), 3.86 (t, J=7.4 Hz, 2H), 3.13-3.18 (m, 2H), 1.63 (s, 6H), 1.51 (s, 1H), 1.30 (s, 6H). ¹³C NMR (75 MHz, CDCl₃) δ148.6, 127.6, 122.2, 80.2, 60.0, 51.3, 35.5, 29.3, 28.1. Anal. Calcd for C₁₅H₂₁NO₂S: C, 64.48; H, 7.58; N, 5.01, Found: C, 64.45; H, 7.67; N, 4.67. LRMS (APIMS) m/z 297 (MNH₄⁺).

### EXAMPLE 3

2-(1,1,3,3-Tetramethyl-2-(nitrosothio)indan-2-yl)acetic acid

3a. 2-(1,1,3,3-Tetramethyl-2-sulfanylindan-2-yl)ethanenitrile

A solution of n-butyl lithium (2.5 M in hexane, 29.4 mL, 73.4 mmol) was cooled to −78° C. and to it was added dropwise a solution of acetonitrile (3.8 mL, 73.4 mmol) in THF (98 mL). The suspension was stirred at −78° C. for 1 hour and a solution of the product of Example 1c (6 g, 29.4 mmol) in THF (49 mL) was added in one portion. The resulting solution was stirred at −78° C. for 1 hour, quenched with water and the THF was evaporated. The residue was treated with ethyl acetate and then water and the aqueous phase was extracted with more ethyl acetate. The combined organic phase was washed with water, brine, dried over sodium sulfate, filtered and evaporated. The residue was chromatographed twice (ethyl acetate:hexane 1:9 each time) to give the title compound (5 g, 69%). Mp. 113-114° C. ¹H NMR (300 MHz, CDCl₃) δ7.26-7.31 (m, 2H), 7.14-7.19 (m, 2H), 2.83 (m, 2H), 1.85 (s, 1H), 1.55 (s, 6H), 1.44 (s, 6H). ¹³C NMR (75 MHz, CDCl₃) δ147.2, 127.8, 122.3, 118.1, 64.0, 50.0, 29.2, 28.1, 27.3. Anal. Calcd for C₁₅H₁₉NS: C, 73.42; H, 7.80; N, 5.71, Found: C, 73.18; H, 7.75; N, 5.62. LRMS (APIMS) m/z 263 (MNHa⁺).

3b. 2-(1,1,3,3-Tetramethyl-2-sulfanylindan-2-yl)acetic acid

A solution of the product of Example 3a (0.5 g, 2.1 mmol) in HCl (conc, 10 mL) and acetic acid (10 mL) was refluxed for 52 hours. The crude reaction mixture was allowed to cool to room temperature and then extracted with ethyl acetate. The organic phase was washed with water (2×), extracted with saturated sodium bicarbonate and the basic aqueous phase was acidified to pH 2 with concentrated HCl. The resulting solution was then extracted with dichloromethane and the combined organic phase was dried over sodium sulfate, filtered and evaporated to give the title compound (240 mg). The ethyl acetate phase after basification also contained some product which was isolated following drying with sodium sulfate, filtration and evaporation and chromatography (ethyl acetate:hexane 1:1) to give the title compound (120 mg, 360 mg total, 66%). Mp. 159-161° C. ¹H NMR (CDCl₃) δ7.24-7.28 (m, 2H), 7.15-7.19 (m, 2H), 2.97 (s, 2H), 2.06 (s, 1H), 1.58 (s, 6H), 1.42 (s, 6H). ¹³C NMR (CDCl₃) δ177.9, 148.1, 127.4, 122.5, 65.0, 50.9, 41.6, 29.5, 27.5 LRMS (APIMS) m/z 282 (MNH₄⁺).

3c. 2-(1,1,3,3-Tetramethyl-2-(nitrosothio)indan-2-yl)acetic acid

To a solution of tert-butyl nitrite (169 mL, 130 mg, 1.27 mmol) in dichloromethane (4 mL) was added the product of Example 3b (112 mg, 0.42 mmol) in one portion as a solid. The solution was stirred for 45 minutes in the dark and the solvent evaporated. The solid was dissolved in a minimum amount of hot ether and three volumes of hot hexane added. The solution was allowed to stand at 4° C. overnight and the solid collected by filtration to give the title compound (75 mg, 57%). ¹H NMR (300 MHz, CDCl₃) δ7.20-7.26 (m, 2H), 7.10-7.15 (m, 2H), 3.89 (s, 2H), 1.63 (s, 6H), 1.61 (s, 6H). ¹³C NMR (75 MHz, CDCl₃) δ148.1, 127.7, 122.4, 52.0, 37.1, 29.6, 27.7. Anal. Calcd for C₁₅H₁₉NO₃S: C, 61.41; H, 6.53; N, 4.77, Found: C, 61.19; H, 6.70; N, 4.50. LRMS (APIMS, -ve scan) m/z 292 (M-H⁻).

### EXAMPLE 4

2-(1,1,3,3-Tetramethyl-2-(nitrosothio)indan-2-yl)ethanenitrile

4. 2-(1,1,3,3-Tetramethyl-2-(nitrosothio)indan-2-yl)ethanenitrile

To a solution of tert-butyl nitrite (325 mL, 251 mg, 2.4 mmol) in dichloromethane (3 mL) was added the product of Example 3a (200 mg, 0.82 mmol) dropwise as a solution in dichloromethane (2 mL). The resultant solution was stirred in the dark for 40 minutes. The solvent evaporated and the residue chromatographed (ethyl acetate:hexane 1:9). The fractions containing the product were pooled, reduced by evaporation and hexane added. After standing overnight at 4° C., the solid was filtered to give the title compound (0.1 g, 45%). Mp. 67-69° C. ¹H NMR (300 MHz, CDCl₃) δ7.30-7.38 (m, 2H), 7.21-7.28 (m, 2H), 3.86 (s, 2H), 1.72 (s, 6H), 1.43 (s, 6H). ¹³C NMR (75 MHz, CDCl₃) δ147.1, 128.4, 122.4, 117.6, 73.8, 51.6, 30.1, 27.1, 24.5. LRMS (APIMS) m/z 292 (MNH₄⁺).

### EXAMPLE 5

2-((N-(2-Methyl-2-(nitrosothio)propyl)carbamoyl)methylthio)acetic acid

5a. 2-((N-(2-Methyl-2-(sulfanylpropyl)carbamoyl)methylthio)acetic acid

To 1-amino-2-methyl-2-propanethiol hydrochloride (1.69 g,11.9 mmole) in dicloromethane (20 mL) at 0° C. was added triethyl amine (1.81 g, 17.9 mmol) followed by thiodiglycolic anhydride (1.43 g, 10.8 mmol). The reaction mixture was stirred at 0° C. for 1 hour and then warmed to ambient temperature overnight. The solvent was removed in vacuo to give a white solid. The solid was re-dissolved in ethyl acetate and washed with water and brine. The organic layer was dried over sodium sulfate, filtered and the solvent removed in vacuo to give the title compound (2.35 g, 94%) as an off white solid. Mp 81-84° C.; ¹H NMR (CDCl₃) δ9.21 (bs, 1H), 7.38 (bs, 1H), 2.96 (s, 2H), 2.95 (s, 2H), 2.92 (d, J=6.3Hz, 2H), 1.59 (s, 1H), 0.94 (s, 6H); LRMS (APIMS) m/z 238 (MH⁺).

5b. 2-((N-(2-Methyl-2-(nitrosothio)propyl)carbamoyl)methylthio)acetic acid

To the product of Example 5a (1.12 g, 4.72 mmol) in methylene chloride (25 mL) at ambient temperature was added tert-butyl nitrite (511 mg, 4.95 mmol). The reaction was stirred at ambient temperature for 2 hours. The reaction mixture was diluted with methylene chloride and washed water (2×). The combined aqueous layers were extracted with methylene chloride (3×) and the combined organic extracts were dried over sodium sulfate. The reaction mixture was filtered and the solvent removed in vacuo to give the title compound (775 mg, 62%) as a red solid. Mp 47-51° C.; ¹H NMR (CDCl₃) δ9.19 (bs, 1H), 7.36 (bs, 1H), 4.06 (d, J=6.4 Hz, 2H), 3.37 (s, 2H), 3.25 (s, 2H), 1.88 (s, 6H); LRMS (APIMS) m/z 267 (M+1)⁺.

### EXAMPLE 6

Nitrosothio(1,3,3-trimethyl-2-prop-2-enylbicyclo(2.2.1)hept-2-yl

6a. 1.3.3-Trimethylbicyclo(2.2.1)heptan-2-one hydrazone

A mixture of 1.3.3-trimethylbicyclo(2.2.1)heptan-2-one (30 g, 197 mmol), hydrazine hydrate (45 g, 881 mmol) acetic acid (12 mL) in ethanol (80 mL) was refluxed gently for 23 hours. The reaction mixture was cooled to room temperature. The ethanol was evaporated and the residue diluted with ether and water. The organic phase was washed with 10% sodium hydroxide solution, brine, dried over magnesium sulfate and filtered. Evaporation of the solvent gave the title compound (30 g, 91%). LRMS (APIMS) m/z 167 (MH⁺).

6b. 1,3,3-Trimethylbicyclo(2.2.1)heptane-2-thione

This was prepared according to the procedure of Okazaki et al., Tetrahedron Lett., 20: 3673-3676, 1979. A solution of triethylamine (45 mL, 323 mmol) in benzene (300 mL) was cooled to 0° C. To the reaction mixture was added, at the same rate, separate solutions of the product of Example 6a (24.4 g, 147 mmol)-and sulphur monochloride (12.4 mL, 154 mmol) each in benzene (120 mL) at 0° C. At the end of the addition, the reaction mixture was stirred at room temperature for 30 minutes and the solid was filtered. The filtrate was washed with water, brine and dried over magnesium sulfate. The residue after filtration and evaporation was chromatographed (neat hexane) and then concentrated by evaporation. The resulting solid was removed by filtration and the filtrate evaporated to give the title compound (17.4 g, 70%). ¹H NMR (300 MHz, CDCl₃) δ2.31 (s, 1H), 1.55-1.90 (m, 5H), 1.31 (s, 3H), 1.18 (s, 3H), 1.15 (s, 3H), 0.90-1.20 (m, 1H).

6c. 1,3,3-Trimethyl-2-prop-2-enylbicyclo(2.2.1)heptane-2-thiol

A solution of the product of Example 6b (10.9 g, 65 mmol) in ether (150 mL) was treated with allylmagnesium bromide (1M in ether, 100 mL, 100 mmol) dropwise at room temperature. After the addition was complete, the reaction mixture was stirred at room temperature for 1 hour, cooled in an ice bath and quenched carefully with 1N HCl. The organic phase was washed with water, brine and dried over sodium sulfate. The residue after filtration and evaporation was chromatographed (neat hexane) to give the title compound (9.3 g, 68%). ¹H NMR (300 MHz, CDCl₃) δ6.04-6.13 (m, 1H), 5.03-5.10 (m, 2H), 2.62-2.72 (m, 1H), 2.30-2.40 (m, 1H), 2.15-2.27 (m, 1H), 1.80-1.90 (m, 1H), 1.67-1.79 (m, 2H), 1.31-1.47 (m, 1H), 1.20 (s, 1H), 1.15 (s, 3H), 1.13 (s, 3H), 1.08 (s, 3H), 1.05-1.22 (m, 2H). ¹³C NMR (75 MHz, CDCl₃) δ138.3, 116.9, 63.5, 54.1, 50.8, 45.2, 44.6, 40.6, 35.0, 28.3, 27.2, 24.8, 18.2.

6d. Nitrosothio(1,3,3-trimethyl-2-prop-2-enylbicyclo(2.2.1)hept-2-yl)

A solution of the product of Example 6c (80 mg, 0.38 mmol) in hexane (2 mL) was added to a solution of tert-butyl nitrite (68 mL, 0.57 mmol) in hexane (2 mL). The reaction mixture was stirred at room temperature in the dark for 30 minutes, and then additional tert-butyl nitrite (20 μL) was added. The reaction mixture was stirred for an additional 1 hour at room temperature in the dark. The solvent evaporated and the residue was chromatographed (neat hexane) to give the title compound (60 mg, 66%). ¹H NMR (300 MHz, CDCl₃) δ5.81-5.90 (m, 1H), 4.84-4.93 (m, 2H), 3.25-3.43 (m 2H), 2.14 (d, J=10.5 Hz, 1H), 1.61-1.82 (m, 3H), 1.50-1.60 (m, 1H), 1.40 (s, 3H), 1.24 (s, 3H), 1.20-1.38 (m, 2H), 0.94 (s, 3H). ¹³C NMR (75 MHz, CDCl₃) δ137.1, 116.3, 55.4, 50.6, 48.4, 42.1, 39.9, 34.1, 28.2, 25.2, 25.1, 19.5.

### EXAMPLE 7

2-(1,3,3-Trimethyl-2-(nitrosothio)bicyclo(2.2.1)hept-2-yl)ethan-1-ol

7a. Phenyl(1,3,3-trimethyl-2-prop-2-enylbicyclo(2.2.1)hept-2-ylthio)methane

A solution of the product of Example 6c (10.1 g, 48.1 mmol) in THF (250 mL) was treated in one portion with sodium hydride (1.34 g of 95%, 53 mmol). After 10 min, benzyl bromide (5.8 mL, 48 mmol) was added slowly and the reaction mixture was stirred at room temperature for 3 hours. Water (100 mL) was added and the THF was removed by evaporation. The aqueous phase was extracted with ethyl acetate and the combined organic phase was washed with brine and dried over magnesium sulfate. The residue after filtration and evaporation was chromatographed twice (hexane then hexane followed by dichloromethane) to give the title compound (10.2 g, 71%). ¹H NMR (300 MHz, CDCl₃) δ7.21-7.31 (m, 5H), 6.31-6.49 (m, 1H), 5.08-5.19 (m, 2H), 3.63 (dd, J=36.8 and 10.5, 2H), 2.62-2.79 (m, 2H), 2.40-2.51 (m,1H), 1.73-1.91 (m, 2H), 1.38-1.60 (m, 3H), 1.25 (s, 3H), 1.20 (s, 3H), 1.17 (s, 3H), 1.15-1.30 (m, 1H).

7b. 2-(1,3,3-Trimethyl-2-(phenylmethylthio)bicyclo(2.2.1)hept-2-yl)ethanal

A solution of the product of Example 7a (10.2 g, 34 mmol) in a mixture of acetone (370 mL) and water (40 mL) was treated with N-methylmorpholine oxide (50% in water, 35 mL, 170 mmol) followed by osmium tetroxide (4% in water, 10.3 mL, 1.7 mmolusing the procedure of Example 2b to give the title compound (5.3 g, 51%). ¹H NMR (300 MHz, CDCl₃) δ10.08 (t, J=2.4 Hz, 1H), 7.19-7.32 (m, 5H), 3.65 (q, J=10.7 Hz, 2H), 2.85 (d, J=2.5 Hz, 2H), 2.34-2.46 (m, 1H), 1.73-1.86 (m, 2H), 1.67 (d, J=4.3 Hz, 1H), 1.42-1.57 (m, 2H), 1.29 (s, 3H), 1.25 (s, 3H), 1.20-1.30 (m, 1H), 1.12 (s, 3H). LRMS (APIMS) m/z 303 (MH⁺).

7c. 2-(1,3,3-Trimethyl-2-(phenylmethylthio)bicyclo(2.2.1)hept-2-yl)ethan-1-ol

A suspension of the product of Example 7b (5.3 g, 17.4 mmol) in methanol (70 mL) was treated with sodium borohydride (0.67 g, 17.4 mmol) in one portion. The reaction mixture was stirred at room temperature for 30 minutes. The solvent was removed by evaporation and the residue was suspended in ethyl acetate, washed with water, brine and dried over sodium sulfate. The residue after filtration and evaporation was chromatographed (ethyl acetate:hexane 1:4 then 1:3) to give the title compound (4.43 g, 84%). ¹H NMR (300 MHz, CDCl₃) δ7.21-7.33 (m, 5H), 3.95-4.06 (m, 1H), 3.80-3.91 (m, 1H), 3.75 (d, J=2.4 Hz, 2H), 2.43-2.56 (m, 1H), 2.22-2.32 (m, 1H), 2.00-2.19 (m, 2H), 1.72-1.83 (m, 2H), 1.36-1.53 (m, 2H), 1.20 (s, 3H), 1.18 (s, 3H), 1.11 (s, 3H), 1.10-1.30 (m, 2H). LRMS (APIMS) m/z 305 (MH⁺).

7d. 2-(1,3,3-Trimethyl-2-sulfanylbicyclo(2.2.1)hept-2-yl)ethan-1-ol

A solution of the product of Example 7c (4.4 g, 14.5 mmol) in ether (5 mL) was treated with liquid ammonia followed by the addition of sodium (approx 1 g) until a permanent blue colour was obtained. The final reaction mixture was stirred for 45 minutes and then ammonium chloride was added to disperse the blue colour. The ammonia was allowed to evaporate and the residue was partitioned between ether and water. The organic phase was washed with more water, brine and dried over sodium sulfate. The residue after filtration and evaporation was chromatographed twice (ethyl acetate:hexane 1:4) to give the title compound (2.8 g, 88%). Mp. 55-60° C. ¹H NMR (300 MHz, CDCl₃) δ3.80-3.93 (m, 2H), 2.16-2.39 (m, 2H), 1.95 (br s, 1H), 1.50-1.82 (m, 5H), 1.34-1.47 (m, 1H), 1.11 (s, 3H), 1.05 (s, 3H), 1.03 (s, 3H), (s, 3H), 100-1.23 (m, 3H). ¹³C NMR (75 MHz, CDCl₃) δ64.01, 62.2, 54.4, 50.6, 44.8, 43.7, 40.6, 34.4, 28.2, 26.2, 24.6, 18.1. LRMS (APIMS) m/z 232 (MNH₄⁺).

7e. 2-(1,3,3-Trimethyl-2-(nitrosothio)bicyclo(2.2.1)hept-2-yl)ethan-1-ol

A solution of the product of Example 7d (0.5 g, 2.33 mmol) in a mixture of methanol (5 mL) and dichloromethane (5 mL) was cooled over ice and then treated slowly with tert-butyl nitrite (1 mL, 7.5 mmol). The reaction mixture was stirred at 0° C. for 15 min and then at room temperature for 30 minutes. The solvent was evaporated and the residue was chromatographed (ethyl acetate:hexane 1:4) to give the title compound (0.51 g, 90%). Mp. 81-85° C. ¹H NMR (300 MHz, CDCl₃) δ3.50-3.71 (m, 2H), 2.91-3.14 (m, 1H), 1.74-1.86 (m, 1H), 2.09-2.19 (m, 1H), 1.35 (s, 3H), 1.24 (s, 3H), 1.20-1.83 (m, 7H), 0.92 (s, 3H). ¹³C NMR (75 MHz, CDCl₃) δ73.3, 61.8, 55.6, 50.6, 48.3, 42.0, 38.8, 33.8, 28.1, 25.1, 25.0, 19.3. LRMS (APIMS) m/z 261 (MNH₄⁺).

### EXAMPLE 8

2-(1,3,3-Trimethyl-2-(nitrosothio)bicyclo(2.2.1)hept-2-yl)ethanenitrile

8a. 2-(1,3,3-Trimethyl-2-sulfanylbicyclo(2.2.1)hept-2-yl)ethanenitrile

A solution of n-butyl lithium (2.5 M in hexane, 29.7 mL, 74.3 mmol) was cooled to -78° C. and then treated with a solution of acetonitrile (3.9 mL, 74.3 mmol) in THF (98 mL). The solution was stirred at −78° C. for 1 hour and then treated with a solution of the product of Example 6b (5 g, 29.7 mmol) in THF (50 mL). The reaction mixture was stirred at −78° C. for 1 hour and then warmed to room temperature over 1 hour. Water (50 mL) was added carefully and the THF was removed by evaporation. The residue was diluted with more water and extracted with ether. The combined organic phase was washed with water, brine and dried over sodium sulfate. The residue after filtration and evaporation was chromatographed (ethyl acetate:hexane 1:9) to give the title compound. Mp. 170-171° C. ¹H NMR (300 MHz, CDCl₃) δ2.72 (q, J=16.6 Hz, 2H), 2.13-2.25 (m, 1H), 1.67-1.78 (m, 3H), 1.67 (s, 1H), 1.37-1.50 (m, 1H), 1.26 (s, 3H), 1.21 (s,3H), 1.19-1.30 (m, 2H), 1.10 (s, 3H). ¹³C NMR (CDCl₃) δ119.9, 60.5, 53.7, 50.1, 45.1, 40.6, 34.3, 30.9, 26.8, 26.3, 24.8, 17.8. LRMS (APIMS) m/z 227 (MNH₄⁺).

8b. 2-(1,3,3-Trimethyl-2-(nitrosothio)bicyclo(2.2.1)hept-2-yl)ethanenitrile

To a solution of the product of Example 8a (70 mg, 0.33 mmol) in dichloromethane (5 mL) was added tert-butyl nitrite (130 μL, 1 mmol) and the reaction mixture was stirred at room temperature in the dark for 2 hours. Additional tert-butyl nitrite (40 μL, 0.31 mmol) was added and the solution was stirred an additional 30 minutes in the dark. The solvent was evaporated and the residue was chromatographed on a preparative plate (ethyl acetate:hexane 1:4) to give the title compound (60 mg, 76%). ¹H NMR (300 MHz, CDCl₃) δ3.66 (dd, J=58.0 and 17.0 Hz, 2H), 2.10-2.20 (m, 1H), 1.95 (br s, 1H), 1.53-1.75 (m, 3H), 1.50 (s, 3H), 1.29 (s, 3H), 1.21-1.40 (m, 2H), 1.01 (s, 3H). ¹³C NMR (75 MHz, CDCl₃) δ118.8, 70.1, 55.0, 50.2, 48.0, 41.6, 33.6, 27.2, 25.8, 25.5, 25.0, 18.6. LRMS (APIMS) m/z 256 (MNH₄⁺).

### EXAMPLE 9

(4-Methoxyphenyl)-N-(2-(1,3,3-trimethyl-2-(nitrosothio)bicyclo(2.2.1)hept-2-yl) ethyl)carboxamide

9a. 2-(2-Aminoethyl)-1,3,3-trimethylbicyclo(2.2.1)heptane-2-thiol

To a solution of the product of Example 8a (2.9 g, 13.7 mmol) in THF (20 mL) was added a solution of lithium aluminum hydride (1M in THF, 21 mL, 21 mmol). The reaction mixture was refluxed for 1.5 hours. The solution was cooled to 0° C. and sodium sulfate decahydrate was added to decompose excess reducing agent. The solid was removed by filtration and washed with dichloromethane:methanol (100 mL, 4:1). The combined filtrate was dried over sodium sulfate, filtered and evaporated. The residue was chromatographed (hexane:ether 1:19) and the solid was recrystalised from ether:hexane (1:1) to give the title compound (1.2 g, 41%). Mp. 42-43° C. ¹H NMR (300 MHz, CDCl₃) δ2.95-3.06 (m, 1H), 2.82-2.92 (m, 1H), 2.22-2.35 (m, 1H), 1.91-2.02 (m, 1 H), 1.70-1.80 (m, 1H), 1.57-1.69 (m, 3H), 1.30-1.48 (m, 4H), 1.10 (s, 6H), 1.02-1.20 (m, 2H), 1.02 (s, 3H). ¹³C NMR (75 MHz, CDCl₃) δ64.0, 54.4, 50.7, 44.8, 43.7, 41.2, 40.5, 34.6, 28.0, 26.4, 24.7, 18.2. LRMS (APIMS) m/z 214 (MH⁺).

9b. (4-Methoxyphenyl)-N-(2-(1,3,3-trimethyl-2-sulfanylbicyclo(2.2.1)hept-2-yl)ethyl) carboxamide

A solution of 4-dimethylaminopyridine (5 mg, 47 μmol), the product of Example 9a (0.1 g, 0.47 mmol) and 4-methoxybenzoic acid (78 mg, 0.52 mmol) in DMF (1 mL) was treated with 1-(3-(dimethylanino)propyl)-3-ethylcarbodiimide hydrochloride (99 mg, 0.52 mmol). The reaction mixture was stirred at room temperature overnight, diluted with ethyl acetate, washed with water, brine and then dried over sodium sulfate. The residue, after filtration and evaporation, was chromatographed (ethyl acetate:hexane 1:2) to give the title compound (73 mg, 45%). ¹H NMR (300 MHz, CDCl₃) δ7.73 (d, 2H), 6.98 (d, 2H), 6.55 (t, 1H), 3.83 (s, 3H), 3.72-3.85 (m, 1H), 3.51-3.62 (m, 1H), 2.14-2.38 (m, 2H), 1.60-1.80 (m, 4H), 1.31-1.46 (m, 1H), 1.12 (s, 3H), 1.11 (s, 3H), 1.10-1.20 (m, 3H), 1.01 (s, 3H). ¹³C NMR (75 MHz, CDCl₃) δ166.7, 162.0, 128.6, 127.0, 113.6, 64.3, 55.5, 54.5, 50.8, 44.8, 41.3, 40.6, 39.5, 34.8, 28.2, 26.3, 24.7, 18.2.

9c. (4-Methoxyphenyl)-N-(2-(1,3,3-trimethyl-2-(nitrosothio)bicyclo(2.2.1)hept-2-yl)ethyl) carboxamide

To a solution of tert-butyl nitrite (89 μL, 68 mg, 0.66 mmol) in dichloromethane (2 mL) was added dropwise, a solution of the product of Example 9b (66 mg, 0.19 mmol) in dichloromethane (1 mL). The reaction mixture was stirred at room temperature in the dark for 40 minutes. The solvent was evaporated and the residue chromatographed (ethyl acetate:hexane 1:2) to give the title compound (32 mg, 45%). ¹H NMR (300 MHz, CDCl₃) δ7.68 (d, 2H), 6.90 (d, 2H), 6.00 (br s, 1H), 3.85 (s, 3H), 3.35-3.57 (m, 2H), 2.76-2.99 (m, 2H), 2.15 (d, 1H), 1.62-1.88 (m, 4H), 1.45-1.62 (m, 1H), 1.45 (s, 3H), 1.31 (s, 3H), 1.15-1.4 (m, 1H), 0.96 (s, 3H)., ¹³C NMR (75 MHz, CDCl₃) δ166.8, 162.1, 128.5, 126.6, 113.7, 74.3, 55.7, 55.3, 50.7, 48.5, 42.1, 39.2, 36.1, 34.0, 28.2, 25.1, 25.0, 19.4. LRMS (APIMS) m/z 377 (MH⁺).

### EXAMPLE 10

Nitrosothio(1,7,7-trimethyl-2-prop-2-enylbicyclo(2.2.1)hept-2-yl

10a. 1,7,7-Trimethyl-2-prop-2enylbicyclo(2.2.1)heptane-2 thiol

A solution of (IR)-(−)-thiocamphor (0.5 g, 2.97 mmol) in ether (10 mL) cooled to 0° C. was treated with allylmagnesium bromide (1M in ether, 4.5 mL, 4.5 mmol) and the reaction mixture was stirred at 0° C. for 30 minutes. Excess cold 2N HCl was added carefully and the solution was extracted with ether. The organic phase was washed with water, brine, dried over magnesium sulfate, filtered and evaporated. The residue was chromatographed (neat hexane) to give the title compound (0.5 g, 80%). ¹H NMR (300 MHz, CDCl₃) 65.91-6.05 (m, 1H), 5.10-5.17 (m, 2H), 2.46-2.54 (m, 2H), 2.18-2.30 (dt, 1H), 2.10 (s, 1H), 1.68-1.75 (m, 3H), 1.46-1.58 (m, 3H), 1.16 (s, 3H), 0.99 (s, 3H), 0.90 (s, 3H). ¹³C NMR (75 MHz, CDCl₃) δ136.2, 117.9, 55.4, 52.7, 50.7, 49.7, 47.9, 45.7, 31.3, 27.1, 22.1, 21.4, 14.3.

10b. -Nitrosothio(1,7,7-trimethyl-2-prop-2-enylbicyclo(2.2.1)hept-2-yl)

A solution of the product of Example 10a (100 mg, 0.48 mmol) in hexane (5 mL) was treated dropwise with tert-butyl nitrite (113 μL, 0.95 mmol). The reaction mixture was stirred at room temperature for 1.5 hours. The solvent was evaporated and the residue chromatographed (neat hexane) to give the title compound (80 mg, 70%). ¹H NMR (300 MHz, CDCl₃) δ5.74-5.83 (m 1H), 4.99-5.06 (m, 2H), 3.34-1.13 (m, 2H), 2.64 (d, J=13.9 Hz, 1H), 2.02-2.15 (m, 2), 1.82-1.96 (m, 2H), 1.62-1.75 (m, 1H), 1.37-1.47 (m, 1H), 0.97 (s, 3H), 0.95 (s, 3H), 0.93 (s, 3H). ¹³C NMR (75 MHz, CDCl₃) δ135.6, 117.7, 68.8, 54.7, 50.8, 46.5, 45.8, 45.5, 31.6, 27.1, 21.5, 21.3, 13.5.

### EXAMPLE 11

4-Aza-4-(2-methyl-2-(nitrosothio)propyl)tricyclo(5.2.1.0<2,6>)dec-8-ene-3,5-dione

11a. 4-Aza-4-(2-methyl-2-sulfanylpropy)tricyclo(5.2.1.0<2,6>)dec-8-ene-3,5-dione

Potassium hydroxide solution (16 M, 3.6 mL, 57.0 mmol) was shaken with a suspension of 2-mercapto-2-methyl-1-propylamine hydrochloride (6.72 g, 47.4 mmol) in ethyl acetate (200 mL). The ethyl acetate solution was separated, dried over sodium sulfate, filtered, and evaporated to give 2-mercapto-2-methyl-1-propylamine (2.70 g, 25.7 mmol, 54%). This was then dissolved in acetic acid (25 mL) and 4-oxatricyclo(5.2.1.0<2,6>dec-8-ene-3,5-dione (4.17 g 25.4 mmol) was added. The reaction was stirred at 100° C. for 1 hour and then cooled to room temperature. The solid was collected by filtration, washed with acetic acid, a small volume of methanol, and dried to give the title compound (2.22 g, 35%). The filtrate was evaporated, treated with toluene and evaporated (repeat four times). The residue was dissolved in dichloromethane and filtered through silica gel to give additional product (2.47 g) contaminated with a small amount of 4-oxatricyclo (5.2.1.0<2,6>dec-8-ene-3,5-dione. ¹ H NMR (300 MHz, CDCl₃) δ6.16 (s, 2H), 3.52 (s, 2H), 3.42 (s, 2H), 3.32 (s, 2H), 1.86 (s, 1H), 1.76 (d, J=8.77 Hz, 1H), 1.57 (d, J=8.77 Hz, 1H), 1.30 (s, 6H). ¹³C NMR (75 MHz, CDCl₃) δ177.9, 134.8, 52.5, 51.0, 45.8, 45.24, 45.0, 30.9. LRMS (APIMS) m/z 252 (MH⁺).

11b. 4-Aza-4-(2-methyl-2-(nitrosothio)propyl)tricyclo(5.2.1.0<2,6>)dec-8-ene-3,5-dione

To a solution of the product of Example 11a (83 mg, 0.33 mmol) in dichloromethane (3 mL) was added tert-butyl nitrite (39 μL, 34 mg, 0.33 mmol). The resulting solution was stirred at room temperature for 1 hour in the dark, then evaporated and the residue chromatographed (ethyl acetate:hexane 1:1) to give the title compound (75 mg, 81%). ¹H NMR (300 MHz, CDCl₃) δ6.12 (s, 2H), 4.10 (s, 2H), 3.41 (s, 2H), 3.30 (s, 2H), 1.82 (s, 6H), 1.75 (d, J=8.8 Hz, 1H), 1.57 (d, J=8.8 Hz, 16H). ¹³C NMR (75 MHz, CDCl₃) δ177.7, 134.7, 56.7, 52.4, 48.0, 45.8, 45.0, 27.5. LRMS (APIMS) m/z 298 (MNH₄⁺).

### EXAMPLE 12

2-(2-(Nitrosothio)adamantan-2-yl)acetamide

12a. tert-Butyl-2-(2-sulfanyladamant-2-yl)acetate

To tert-butyl acetate (25 mL, 21.6 g, 186 mmol) in THF (400 mL) at −78° C. was added lithium diisopropylamide monotetrahydrofuran (1.5 M solution in cyclohexane, 100 mL, 150 mmol). The solution was stirred at −78° C. for 40 min and 2-adamantanethione (21.9 g, 131.6 mmol) in THF (400 mL) was added. The reaction was stirred at room temperature for 2 hours, diluted with dichloromethane and HCl (2N, 75 mL). The organic phase was removed, washed with brine, dried over magnesium sulfate, filtered, and evaporated. The residue was chromatographed (ethyl acetate:hexane 1:19) to give the title compound (34.7 g, 93%). ¹H NMR (300 MHz, CDCl₃) δ2.87 (s, 2H), 2.47 (d, J=11.5, 2H), 2.38 (s, 1H), 2.11 (d, J=11.9, 2H), 1.98 (s, 2H), 1.96 (m, 2H), 1.84-1.96 (m, 6H), 1.47 (s, 9H). ¹³C NMR (75 MHz, CDCl₃) δ170.8, 80.7, 54.0, 47.2, 38.9, 38.1, 33.9, 33.23, 28.1, 27.4, 26.8. LRMS (APIMS) m/z 283 (MH⁺). Anal. Calcd for C₁₆H₂₆O₂S: C, 68.04; H, 9.28. Found: C, 68.14; H, 9.30.

12b. 2-(2-sulfanyladamantan-2-yl)acetic acid

Trifluoroacetic acid (30 mL, 390 mmol) was added dropwise to a stirred suspension of the product of Example 12a (20 g, 70 mmol) in dichloromethane (200 mL). The mixture was stirred at room temperature for 2 hours and the volatile material evaporated. The residue was dissolved in a minimum amount of warm ethyl acetate and then hexane (50 mL) was slowly added. The solvent was evaporated to half of its volume and stored at 4° C. Filtration gave the title compound (10.5 g, 66%). Mp 178-180° C. ¹H NMR (300 MHz, CDCl₃) δ9.5 (br s, 1H), 3.04 (s, 2H), 2.49 (d, J=11.2 Hz, 2H), 2.25 (s, 1H), 2.1-2.0 (m, 4H), 1.9 (m, 2H), 1.7-1.6 (m, 6H). ¹³C NMR (75 MHz, CDCl₃) δ177.7, 53.4, 46.3, 38.9, 37.8, 33.8, 33.2, 27.4, 26.8. LRMS (APIMS, -ve scan) m/z 225 (M-H⁻). Anal. Calcd for C₁₂H₁₈O₂S: C, 63.68; H, 8.02. Found: C, 63.40; H, 7.90.

12c. 2-(2-Sulfanyladamantan-2-yl)acetamide

The product of Example 12b (2.28 g, 10 mmol) and 1-(3-(dimethylamino)propyl)-3-ethylcarbodiimide hydrochloride (1.96 g, 10.2 mmol) in methanol (40 mL) was stirred at room temperature for 1 hour. After cooling to 0° C., ammonia gas was introduced to give a saturated solution which was stirred at room temperature overnight. The solvent was evaporated and the residue treated with methanol and then evaporated (repeat one more time). The residue was triturated with water. The resulting solid was collected by filtration, washed with water, and dried. The solid was chromatographed (ethyl acetate:hexane 1;1) to give the title compound (1.8 g, 83%). ¹H NMR (300 MHz, CDCl₃) δ6.16 (br s, 1H), 5.78 (br s, 1H), 2.90 (s, 2H), 2.51-2.60 (m, 2H), 2.23 (s, 1H), 2.11-2.19 (m, 2H), 1.94-1.98 (m, 2H), 1.881.93 (m, 2H), 1.7-1.6 (m, 6H). ³C NMR (75 MHz, CDCl₃) δ173.2, 54.5, 47.9, 39.0, 37.9, 33.9, 33.4, 27.5, 26.9. LRMS (APIMS) m/z 226 (MH⁺), 243 (M+NH₄⁺).

12d. 2-(2-(Nitrosothio)adamantan-2-yl)acetamide

To a solution of the product of Example 12c (1.38 g, 6.13 mmol) in dichloromethane (100 mL) in an ice-water bath was added tert-butyl nitrite (3.00 mL, 2.6 g, 25.2 mmol). The solution was stirred at 0° C. for 20 min. The solvent was evaporated and the residue chromatographed (ethyl acetate:hexane 1:3) to give the title compound (1.27 g, 82%). ¹H NMR (300 MHz, CDCl₃) δ5.58 (br s, 1H), 5.32 (br s, 1H), 3.63 (m, 2H), 2.80 (s, 2H), 2.44 (m, 2H), 2.08 (m, 4H), 1.9-1.6 (m, 6H). ¹³C NMR (75 MHz, CDCl₃) δ172.3, 66.6, 43.2, 38.8, 35.6, 33.71, 33.14, 27.06, 27.00. LRMS (APIMS) m/z 255 (MH⁺).

### EXAMPLE 13

(1,1-Bis(tert-butyl)but-3-enyl)nitrosothio

13a. 3-(tert-Butyl)-2,2-dimethylhex-5-ene-3-thiol

2,2,4,4-Tetramethylpentane-3-thione (8.35 g, 53 mmol) in ether (150 mL) was cooled to 0° C. and then treated with allylmagnesium bromide (1M in ether, 120 mL, 120 mmol) dropwise. The resultant solution was stirred over ice for 30 minutes, quenched carefully with excess cold 2N HCl and then extracted with ether. The combined organic phase was washed with brine, dried over sodium sulfate, filtered and evaporated to give the title compound (9.0 g, 85%) that was used in the next step without purification. ¹H NMR (300 MHz, CDCl₃) δ6.04-6.16 (m, 1H), 4.99-5.06 (m, 2H), 2.56-2.59 (m, 2H), 1.40 (s, 1H), 1.19 (s, 18H). ¹³C NMR (75 MHz, CDCl₃) δ139.3, 116.1, 64.9, 42.6, 41.3, 30.4. Anal. Calcd for C₁₂H₂₄S: C, 71.93; H, 12.07, Found: C, 72.04; H, 11.94.

13b. (1,1-Bis(tert-butyl)but-3-enyl)nitrosothio

A solution of the product of Example 13a (0.25 g, 1.25 mmol) in dichloromethane (3 mL) was treated with tert-butyl nitrite (0.2 mL, 1.5 mmol) and the reaction mixture was stirred at room temperature for 30 minutes. The resulting solution was evaporated and the residue chromatographed (neat hexane) to give the title compound (0.19 g, 67%). ¹H NMR (300 MHz, CDCl₃) δ5.91-6.00 (m, 1H), 4.94-5.13 (m, 2H), 3.53 (dd, J=6.7 and 1.3 Hz, 2H), 1.27 (s, 18H).

### EXAMPLE 14

4-(tert-Butyl)-5,5-dimethyl-4-(nitrosothio)hexan-1-ol

14a. 1,1-Bis(tert-butyl)-1-(phenylmethylthio)but-3-ene

A solution of the product of Example 13a (6.2 g, 31 mmol) in THF (10 mL) was treated with sodium hydride (1.8 g of 60%, 44 mmol) and the reaction mixture was stirred at room temperature for 20 minutes. Benzyl bromide (4 mL, 5.8 g, 34 mmol) was added and the reaction mixture was stirred at room temperature for 1 hour. The solvent was reduced by evaporation and water added carefully. The aqueous phase was extracted with hexane and the organic phase dried over sodium sulfate, filtered and evaporated. The residue was chromatographed (neat hexane then ether:hexane 1:49) to give the title compound (8.83 g, 98%). ¹H NMR (300 MHz, CDCl₃) δ7.22-7.34 (m, 5H), 6.28-6.41 (m, 1H), 5.00-5.11 (m, 2H), 3.81 (s, 2H), 2.75-2.79 (m, 2H), 1.26 (s, 18H). ¹³ C NMR (75 MHz, CDCl₃) 8 140.0, 138.7, 129.1, 128.4, 126.8, 114.9, 67.3, 44.1, 39.0, 37.4, 30.9. Anal. Calcd for C₁₉H₃₀S: C, 78.55; H, 10.41, Found: C, 78.60; H, 10.32.

14b. 4-(tert-Butyl)-5,5-dimethyl-4-(phenylmethylthio)hexan-1-ol

A solution of the product of Example 14a (1.4 g, 4.8 mmol) in hexane (10 mL) was treated with a solution of boranemethylsulphide (1M in dichloromethane, 1.9 mL, 1.9 mmol) and the reaction mixture was stirred at room temperature for 3 hours. An additional amount of boranemethylsulphide (1 mL) was added and the solution was stirred at room temperature for 1 hour. To the reaction mixture was added ethanol (5 mL), 2N NaOH (5 mL) and hydrogen peroxide (50%, 1 mL) and the resulting solution was refluxed for 30 minutes. The solution was cooled to room temperature, diluted with water, extracted with ether and the combined organic phase was dried over sodium sulfate, filtered and evaporated. The residue was chromatographed (ether:hexane 1:1) to give the title compound (0.85 g, 58%). ¹H NMR (300 MHz, CDCl₃) δ7.17-7.32 (m, 5H), 3.81 (s, 2H), 3.54 (t, J=6.9 Hz, 2H), 1.83-2.03 (m, 4H), 1.26 (s, 18H). ¹³C NMR (75 Mz, CDCl₃) δ138.6, 129.0, 128.3, 126.7, 67.8, 63.7, 43.8, 37.3, 31.9, 31.8, 31.0. LRMS (APIMS) m/z 326 (MNH₄⁺).

14c. 4-(tert-Butyl)-5,5-dimethyl-4-sufanylhexan-1-ol

A solution of the product of Example 14b (0.85 g, 2.8 mmol) in ether (5 mL) was treated with liquid ammonia (30 mL) followed by sodium to give a permenant blue solution (approx 0.8 g). Solid ammonium chloride was added to disperse the blue colour and the ammonia was allowed to evaporate. The residue was dissolved in water, acidified with 2N HCl and extracted with ether. The combined organic phase was washed with brine, dried over sodium sulfate, filtered and evaporated to give the title compound as an inseperable mixture with the corresponding disulphide (0.51 g) which was used in the next step without purification.

14d. 4-(tert-Butyl)-5,5-dimethyl-4-(nitrosothio)hexan-1-ol

A solution of the product mixture of Example 14c (0.41) in a combination of dichloromethane (5 mL) and methanol (2 mL) was treated with a solution of HCl in 2-propanol (2 mL) followed by tert-butyl nitrite (1 mL, 774 mg, 7.5 mmol). The reaction mixture was stirred at room temperature for 1 hour. The solvent evaporated and the residue chromatographed (ether:hexane 1:1) to give the title compound (0.2 g). ¹H NMR (300 MHz, CDCl₃) δ3.59 (t, J=6.5 Hz, 2H), 2.64-2.69 (m, 2H), 2.01 (br s, 1H), 1.75-1.85 (m, 2H), 1.27 (s, 18H). ¹³C NMR (75 MHz, CDCl₃) δ63.4, 43.4, 32.2, 31.4, 31.0, 30.8. LRMS (APIMS) m/z 265 (MNH₄⁺).

### EXAMPLE 15

3-(tert-Butyl)-4,4-dimethyl-3-(nitrosothio)pentanenitrile

15a. 3-(tert-Butyl)-4,4-dimethyl-3-sulfanylpentanenitrile

A solution of n-butyl lithium (2.5 M in hexane, 25.3 mL, 63.2 mmol) was cooled to −78° C. and to it was added a solution of acetonitrile (3.3 mL, 63.2 mmol) in THF (98 mL). The reaction mixture was stirred at −78° C. for 1 hour and then a solution of 2,2,4,4-tetramethylpentane-3-thione (prepared exactly as described by Ohno, A.; Nakamura, K.; Nakazima, Y.; Oka, S., Bull. Chem. Soc. Jpn., 48, 2403-2404, 1975) (3.3 g, 20.9 mmol) in THF (49 mL), was added in one portion. The reaction mixture was stirred at room temperature for 1 hour, quenched carefully with 2N HCl and the THF removed by evaporation. The residue was diluted with water and extracted with ethyl acetate. The combined organic phase was washed with brine and dried over sodium sulfate. The residue after filtration and evaporation was chromatographed (ethyl acetate:hexane 1:9) to give the title compound (3.5 g, 84%). Mp. 154-155° C. ¹H NMR (300 MHz, CDCl₃) δ2.86 (s, 2H), 1.66 (s, 1H), 1.29 (s, 18H). ¹³C NMR (75 MHz, CDCl₃) δ119.8, 61.7, 42.2, 29.9, 27.1. LRMS (APIMS) m/z 217 (MNH₄⁺).

15b. 3-(tert-Butyl)-4,4-dimethyl-3-(nitrosothio)pentanenitrile

A solution of the product of Example 15a (200 mg, 1 mmol) in dichloromethane (5 mL) was treated with tert-butyl nitrite (160 mL, 123 mg, 1.2 mmol). The reaction mixture was stirred at room temperature for 30 minutes. The solvent was evaporated and the residue was chromatographed (ethyl acetate:hexane 1:9) to give the title compound (210 mg, 92%). Mp. 92-93° C. ¹H NMR (300 MHz, CDCl₃) δ3.82 (s, 2H), 1.36 (s, 18H). ¹³C NMR (75 MHz, CDCl₃) δ119.5, 73.1, 43.1, 30.3, 24.3. LRMS (APIMS) m/z 246 (MNH₄⁺).

### EXAMPLE 16

(1,1-Diadamantanylbut-3-enyl)nitrosothio

16a. 1,1-Diadamantylmethanimine hydrochloride

The title compound was prepared according to a published procedure as described below. Sodium (5.4 g, 233 mmol) was heated in anhydrous octane (200 mL) at 115° C. (bath temperature) for 10 min. The temperature was adjusted to 100° C. and 1-adamantanecarbonitrile (25 g, 155 mmol) was added. The reaction mixture was stirred at 100° C. for 1 hour and then at 115° C. for 6 hours. The solution was cooled to room temperature and treated with a 3:2 mixture of ethyl acetate:ether (250 mL). 2N HCl was added to give a precipitate which was collected by filtration to give the title compound (17 g, 66%). ¹H NMR (300 MHz, DMSO-d₆) δ11.75 (br s, 2H), 2.01-2.17 (m, 18H), 1.64-1.74 (br s, 12H). ¹³C NMR (75 MHz, DMSO-d₆) δ207.3, 45.4, 38.4, 35.0, 27.4. LRMS (APIMS) m/z 298 (MH⁺, free base).

16b. 1,1-diadamantylketone hydrazone

A solution of the product of Example 16a (1.3 g, 3.9 mmol) in hydrazine hydrate (30 mL) was treated with sulphuric acid (conc, 10 drops) and refluxed gently for 5 d. The reaction mixture was cooled to room temperature, diluted with water, extracted with ether and the combined extracts were dried over sodium sulfate, filtered and evaporated to give the title compound (0.9 g, 75%). ¹H NMR (300 MHz, CDCl₃) δ2.33 (s, 2H), 1.92-2.10 (m, 15H), 1.82 (m, 15H). LRMS (APIMS) m/z 313 (MH⁺).

16c. Diadamantanylmethane-1-thione

A solution of triethylamine (741 μL, 534 mg, 5.3 mmol) in benzene (15 mL) was cooled to 0° C. To this was added separate solutions of the product of Example 16b (0.73 g, 2.4 mmol) in THF (10 mL) and sulphur monochloride (190 μL, 324 mg, 2.4 mmol) in benzene (10 mL) at equal rates. After the addition was complete the mixture was stirred over ice for 5 minutes and then at room temperature for 30 minutes. The reaction mixture was quenched with water and the organic phase washed with water, brine and dried over sodium sulfate. The residue after filtration and evaporation was chromatographed (ether:hexane 1:9) to give the title compound (0.75 g, 100%). ¹H NMR (300 MHz, CDCl₃) δ1.65-2.25 (m, 30H).

16d. 1,1-Diadamantanylbut-3-ene-1-thiol

A solution of the product of Example 16c (560 mg, 1.7 mmol) in ether (20 mL) was cooled to 0° C. and after 10 min a solution of allylmagnesiumbromide (1M in ether, 5.4 mL, 5.4 mmol) was added dropwise. The reaction mixture was stirred over ice for 30 min, quenched carefully with water and the organic phase washed with brine, dried over sodium sulfate, filtered and evaporated. The residue was chromatographed twice (ether:hexane 1:19) to give the title compound (280 mg, 44%). ¹H NMR (300 MHz, CDCl₃) δ6.07-6.21 (m, 1H), 5.03 (dd, J=13.7 and 1.95 Hz, 2H), 2.58 (d, J=6.8 Hz, 2H), 1.60-2.25 (m, 31H). ¹³C NMR (75 MHz, 140.1, 115.8, 66.6, 45.6, 41.4, 39.6, 38.4, 37.0, 36.9, 29.4, 28.9. Anal. Calcd for C₂₄H₃₆S 1.5% H₂O: C, 79.61; H, 10.18, Found: C, 79.59; H, 9.88

16e. (1,1-Diadamantanylbut-3-enyl)nitrosothio

A solution of the product of Example 16d (123 mg, 0.34 mmol) in dichloromethane (2 mL) was added dropwise to a solution of tert-butyl nitrite (137 μL, 106 mg, 1.04 mmol) in dichloromethane (2 mL) and the resulting mixture was stirred at room temperature for 40 min in the dark. The residue after evaporation was chromatographed (ether:hexane 1:19) to give the title compound (85 mg, 64%). ¹H NMR (CDCl₃) δ5.96-6.10 (m, 1H), 4.95-5.17 (m, 2H), 2H), 2.50 (d, J=6.2 Hz, 2H), 1.55-2.30 (m, 30H). ¹³C NMR (CDCl₃) δ139.2, 115.2, 47.1, 40.2, 36.9 29.4, 29.1.

### EXAMPLE 17

3-(N-(2-Methyl-2-(nitrosothio)propyl)carbamoyl)pyrazine-2-carboxylic acid

17a. 3-(N-(2-Methyl-2-sulfanylpropyl)carbamoyl)pyrazine-2-carboxylic acid

A suspension of 2-mercapto-2-methyl-1-propylamine hydrochloride (1.14 g, 8 mmol) in dichloromethane (15 mL) was cooled to 0° C. and then treated with triethylamine (1.23 mL, 0.9 g, 8.9 mmol) followed by furano(3,4-b)pyrazine-5,7-dione (1.2 g, 8 mmol). The reaction mixture was stirred at 0° C. for 40 minutes then at room temperature for 1 hour. The solvent was removed by evaporation and the residue triturated with hexane/ether to give the title compound (1.2 g, 59%). Mp 141-144° C. ¹H NMR (300 MHz, DMSO-d6) δ8.83-8.86 (m, 3H), 3.45 (d, J=6.4 Hz, 2H), 2.87 (s, 1H), 1.33 (s, 6H).

17b. 3-(N-(2-Methyl-2-(nitrosothio)propyl)carbamoyl)pyrazine-2-carboxylic acid

A solution of the product of Example 17a (0.2 g, 0.8 mmol) in a mixture of dichloromethane (3 mL) and methanol (1 mL) was treated with tert-butyl nitrite (310 mL, 0.24 g, 2.35 mmol) and a solution of HCl in ether (25 μL). The reaction mixture was stirred at room temperature for 30 minutes in the dark and the solvent evaporated. The residue was suspended in a solution of HCl in ether and the solid filtered and dried to give the title compound (0.15 g, 67%). ¹H NMR (300 MHz, DMSO-d6) δ9.19 (t, J=6.6 Hz, 1H), 8.84 (dd, J=7.9 and 2.5 Hz, 2H), 4.07 (d, J=6.6 Hz, 2H), 1.91 (s, 6H).

### EXAMPLE 18

(2-Methyl-2-(nitrosothio)propyl)(2-methylthiopyrimnidin-4-yl)amine

18a. 2-Mercapto-2-methyl-1-propylamine

To a suspension of 2-mercapto-2-methyl-1-propylamine hydrochloride (8 g, 56.7 mmol) in ether (100 mL) was added triethylamine (20 mL, 143.5 mmol). The reaction mixture was stirred overnight at room temperature, filtered and the filtrate evaporated to give the product as a volatile solid (3.95 g, 91%). ¹H NMR (CDCl₃) δ2.77 (s, 2H), 1.72 (s, 3H), 1.34 (s, 6H). ¹³C NMR (CDCl₃) 56.2, 46.9, 29.6.

18b. 2-Methyl-1-((2-methylthiopyrimidin-4-yl)amino)propane-2-thiol

A solution of 4-chloro-2-methylthiopyrimidine (1.4 mL, 12.0 mmol) and the product of Example 18a (2.32 g, 22.1 mmol) in pyridine (10 mL) was degassed by 2 freeze-pump-thaw cycles and blanketed with argon. The reaction was heated to 70° C. overnight and the pyridine was evaporated. The resulting mixture was taken up with dichloromethane and washed with water, saturated sodium bicarbonate solution, water, dried over sodium sulfate, filtered, and evaporated. The residue was chromatographed (ethyl acetate:hexane 1:2) to give the title compound (1.7 g, 62%). ¹H NMR (300 MHz, CDCl₃) δ7.95 (d, J=5.9 Hz, 1H), 6.12 (d, J=5.9 Hz, 1H), 5.77 (app. t, J=5.6 Hz, 1H), 3.50 (br s, 2H), 2.49 (s, 3H), 1.79 (s, 1H). 1.39 (s, 6H). ¹³C NMR (75 MHz, CDCl₃) δ171.0, 161.7, 154.7, 100.5, 53.2, 45.4, 29.8, 13.7. LRMS (EI) m/z 230 (MH⁺).

18c. (2-Methyl-2-(nitrosothio)propyl)(2-methylthiopyrimidin-4-yl)amine tert-Butyl nitrite (0.53 mL, 4.49 mmol) was added to an ice-cold solution of the product of Example 18b (0.93 g, 4.06 mmol) in dichloromethane (25 mL) and HCl (1N, 15 mL). The mixture was stirred over ice for 15 minutes and at room temperature for 2 hours in the dark. The reaction mixture was treated with dichloromethane, washed with water, saturated sodium bicarbonate solution, water, dried over sodium sulfate, filtered and evaporated. The residue was chromatographed (ethyl acetate:hexane 1:2) to give the title compound (0.86 g, 75%). Mp. 77-79° C. ¹H NMR (300 MHz, CDCl₃) δ7.95 (d, J=5.9 Hz, 1H), 6.07 (d, J=5.9 Hz, 1), 5.57 (t, J=5.9 Hz, 1H), 4.24 (d, J=5.3 Hz, 2H), 2.49 (s, 3H), 1.91 (s, 6H). ¹³C NMR (75 MHz, CDCl₃) δ171.4, 161.9, 155.0, 100.0, 57.4, 50.5, 26.8, 13.8. LRMS (EI) m/z 259 (MH+). Anal. Calcd for C₉H₁₄N₄OS₂: C, 41.84; H, 5.46; N, 21.69; S, 24.82. Found: C, 41.88; H, 5.67; N, 21.34; S, 24.8

### EXAMPLE 19

4-(N-(2-Methyl-2-(nitrosothio)propyl)carbamoyl)butanoic acid

19a. 4-(N-(2-Methyl-2-sulfanylpropyl)carbamoyl)butanoic acid

To a suspension of 2-mercapto-2-methyl-1-propylamine hydrochloride (11.3 g, 80 mmol) in dichloromethane (80 mL) at 0° C. was added triethylamine (12 mL, 86 mmol). The reaction mixture was stirred at 0° C. for 10 minutes, then glutaric anhydride (9.0 g, 78 mmol) was added. The reaction mixture was stirred at 0° C. for 10 minutes then at room temperature for 2 hours. The solid was removed by filtration, and the filtrate evaporated. The residue was treated with ethyl acetate and filtered again. The filtrate was washed with IN HCl, brine and dried over sodium sulfate. The residue after filtration and evaporation was triturated with ether/hexane to give the title compound (11.7 g, 67%). Mp 101-104° C. ¹H NMR (300 MHz, CDCl₃) δ9.35 (br s, 1H), 6.28 (br s, 1H), 3.30 (d, J=6.15 Hz, 2H), 2.28-2.38 (m, 4H), 1.90-2.00 (m,2H), 1.31 (s, 6H). LRMS (EI) m/z 220 (MH⁺).

19b. 4-(N-(2-Methyl-2-(nitrosothio)propyl)carbamoyl)butanoic acid

A solution of the product of Example 19a (11.5 g, 52.5 mmol) in dichloromethane (700 mL) was cooled to 0° C. and tert-butyl nitrite (10.2 mL, 87 mmol) was added over 15 minutes. The solution was stirred in the dark at 0° C. for 15 minutes, warmed to room temperature over 15 min and stirred at room temperature for 30 min. The solvent was evaporated and the residue was dissolved in ethyl acetate (1 L), washed with water, brine and dried over magnesium sulfate. The residue after filtration and evaporation was triturated with ethyl acetate:hexane 1:4 to give the title compound (11.7 g, 88%). Mp 104-107° C. ¹H NMR (300 MHz, DMSO-d6) δ12.00 (br s, 1H), 8.20 (br s, 1H), 3.81 (d, J=6.4 Hz, 2H), 2.11-2.20 (m, 4H), 1.82 (s, 6H), 1.64-1.73 (m, 2H). ¹³C NMR (75 MHz, DMSO-d6) δ174.6, 172.7, 58.7, 48.6, 34.9, 33.5, 26.9, 21.2. LRMS (EI) m/z 249 (MH⁺).

### EXAMPLE 20

N-(2-Methyl-2-(nitrosothio)propyl)((2-methyl-2-(nitrosothio)propyl)amino) carboxamide

20a. 2-Methyl-2((2,4,6-trimethoxyphenyl)methylthio)propylamine.

A suspension of 2-mercapto-2-methyl-1-propylamine hydrochloride (5.6 g, 40 mmol) in dichloromethane (200 mL) was cooled to 0° C. (internal temperature) and trifluoroacetic acid (61 mL, 90 g, 0.79 mol) introduced. To this was added a solution of 2,4,6-trimethoxybenzyl alcohol (prepared from 2,4,6-trimethoxybenzaldehyde as described by Munson, et al., J. Org. Chem., 57: 3013-3018, 1992) (7.5 g, 38 mmol) in dichloromethane (50 mL) such that the temperature of the solution did not rise above 5° C. Following the addition, the solution was stirred over ice for an additional 10 minutes. The volatile material was evaporated, and the residue was diluted with ethyl acetate, washed with saturated bicarbonate solution, dried over sodium sulfate, filtered and evaporated. The residue was chromatographed (ethyl acetate:hexane 3:1 then neat ethyl acetate then ethyl acetate:methanol 4:1) to give the title compound (4 g, 37%). ¹H NMR (300 MHz, CDCl₃) δ6.09 (s, 2H), 3.84 (s, 6H), 3.73 (s, 3H), 3.69 (s, 2H), 2.70 (s, 2H), 1.91 (s, 2H), 1.29 (s, 6H). ¹³C NMR (75 MHz, CDCl₃) δ160.2, 158.6, 106.8, 90.6, 55.7, 55.2, 50.9, 48.1, 26.2, 19.7. HRMS (EI) m/z C₁₄H₂₃NO₃S requires 285.1399 found 285.1397.

20b. N-(2-Methyl-2-((2,4,6-trimethoxyphenyl)methylthio)propyl)((2-methyl-2-((2,4,6-trimethoxyphenyl)methylthio)propyl)amino)carboxamide

A mixture of the product of Example 20a (1.5 g, 5.2 mmol) and disuccinirnidyl carbonate (673 mg, 2.6 mmol) was refluxed for 10 hours in chloroform (15 mL) and then allowed to cool. The residue after evaporation was chromatographed (ethyl acetate:hexane 4:1) to give the title compound (0.7 g, 45%). ¹H NMR (300 MHz, CDCl₃) δ6.07 (s, 4H), 5.19 (t, J=5.7 Hz, 2H), 3.79 (s, 12H), 3.77 (s, 6H), 3.64 (s, 4H), 3.35 (d, J=5.7 Hz, 4H), 1.31 (s, 12H).

20c. N-(2-Methyl-2-sulfanylpropyl)((2-methyl-2-sulfanylpropyl)amino)carboxade

A mixture the product of Example 20b (0.7 g, 1.17 mmol), phenol (0.2 g), anisole (0.25 mL) and water (0.25 mL) was treated with trifluoroacetic acid (10 mL). The resultant solution was stirred at room temperature for 45 minutes and the solvent was evaporated. The residue was neutralized with saturated sodium bicarbonate solution, and extracted with ethyl acetate. The combined organic phase was dried over sodium sulfate, filtered and the residue, after evaporation, chromatographed (ethyl acetate:hexane 1:3 then 1:1) followed by a single recrystallization from ether to give the title compound (0.19 g, 68%). Mp. 171-173° C. ¹H NMR (300 MHz, CDCl₃) δ5.02 (br s, 2H), 3.31 (d, J=6.2 Hz, 4H), 1.66 (s, 2H), 1.39 (s, 12H). ¹³C NMR (75 MHz, CDCl₃) δ158.5, 53.6, 46.0, 29.9. HRMS (EI) m/z C₉H₂₀N₂OS₂ requires 236.1017 found 236.1009.

20d. N-(2-Methyl-2-(nitrosothio)propyl)((2-methyl-2-(nitrosothio)propyl)amino)carboxamide

To a solution of tert-butyl nitrite (121 μL, 104 mg, 1 mmol) in dichloromethane (2 mL) was added dropwise a solution of the product of Example 20c (80 mg, 0.33 mmol) in dichloromethane (2mL) and the resultant solution was stirred at room temperature in the dark for 30 minutes. The residue, after evaporation of the solvent, was chromatographed (ethyl acetate:hexane 1:1) to give the title compound (15 mg, 15%). ¹H NMR (300MHz, CDCl₃) δ5.22 (t, J=5.9 Hz, 2H), 3.93 (d, J=6.2 Hz, 4H), 1.86 (s, 12H).

### EXAMPLE 21

1-(2-Methyl-2-(nitrosothio)propyl)imidazolidine-2,4,5-trione

21a. Amino-N-(2-methyl-2-sulfanylpropyl)amide

A solution of 2-mercapto-2-methyl-1-propylamine hydrochloride (1 g, 7 mmol) and sodium cyanate (0.46 mg, 7 mmol) in methanol (4 mL) and water (1 mL) was heated to 75° C. (bath temperature) for 3 hours and allowed to cool to room temperature. The solvent was evaporated and the residue treated with chloroform (20 mL) and then stirred for 10 minutes. The filtrate was separated and evaporated to give the title compound (1.1 g, 100%). Mp 105-107° C. ¹H NMR (300 MHz, CDCl₃) δ5.78 (br s, 1H), 4.94 (s, 2H), 3.25 (d, J=6.2 Hz, 2H), 1.72 (s, 1H), 1.35 (s, 6H). ¹³C NMR (75 MHz, CDCl₃) 6159.3, 53.5, 45.8, 29.9. HRMS (EI) m/z C₅H₁₂N₂OS requires 148.0670 found 148.0667. Anal. Calcd for C₅Hl₂N₂OS: C, 40.51; H, 8.16; N, 18.90. Found: C, 40.68; H, 8.08; N, 19.08.

21b. 1-(2-Methyl-2-sulfanylpropyl)imidazolidine-2,4,5-trione

Sodium (0.5 g, 21 mmol) was dissolved in ice-cold methanol (25 mL) and the solution warmed to room temperature. To this was added the product of Example 21a (1.5 g, 10.1 mmol) and, after 5 min, diethyl oxalate (1.5 g, 10.1 mmol) was added dropwise. The resultant solution was stirred at room temperature for 3 hours and then treated with concentrated HCl (3 mL) and filtered. The volatile material was evaporated and the residue chromatographed (ethyl acetate:hexane 1:3 then 1:1) to give the title compound (1.2 g, 59%). Mp. 168-170° C. ¹H NMR (300 MHz, DMSO-d6) δ3.57 (s, 2H), 3.15 (s, 1H), 1.30 (s, 6H). ¹³C NMR (75 MHz, DMSO-d6) δ159.6, 159.5, 155.6, 51.8, 45.2, 30.7. HRMS (EI) m/z C₇H₁₀N₂O₃S requires 202.0412 found 202.0414. Anal. Calcd for C₇H₁₀N₂O₃S: C, 41.57; H, 4.98; N, 13.85. Found: C, 41.82; H, 5.07; N, 13.76.

21c. 1-(2-Methyl-2-(nitrosothio)propyl)imidazolidine-2,4,5-trione

To a solution of tert-butyl nitrite (650 μL, 497 mg, 4.82 mmol) in dichloromethane (10 mL) was added dropwise a solution of the product of Example 21b (650 mg, 3.2 mmol) in DMF (2 mL). The resultant solution was stirred at room temperature in the dark for 30 min. The solvent was evaporated and the residue chromatographed (ethyl acetate:hexane 1:1) to give the title compound (500 mg, 68%). Mp. 80-82° C. ¹H NMR (300 MHz, CDCl₃) δ8.53 (br s, 1H), 4.44 (s, 2H), 1.95 (s, 6H). ¹³C NMR (75 MHz, CDCl₃/DMSO-d6) δ158.0, 157.5, 153.9, 56.0, 48.5, 27.0. LRMS (EI) m/z 230 (M⁺−H). Anal. Calcd for C₇H₉N₃O₄S: C, 36.36; H, 3.92; N, 18.17. Found: C, 36.58; H, 3.85; N, 17.91.

### EXAMPLE 22

3-(5-(1-Methyl-1-(nitrosothio)ethyl)-3,6-dioxopiperizin-2-yl)propanoic acid

22a. tert-Butyl methyl 2-(2-amino-3-((4-methoxyphenyl)methylthio)-3-methylbutanoylamino) pentane-1,5-dioate

To a stirred solution of glutamic acid(O t-Bu)OMe HCl (2.93 g, 11.5 mmol) in chloroform (110 mL) cooled to −78° C. was added triethylamine (4 mL, 28.9 mmol) and a solution of 5-((S-p-methoxybenzyl)2-mercaptoprop-2-yl)oxazolidin-2,4-dione (prepared according to the procedure described in Tetrahedron Lett., 35:1631-1634, 1994) (3.4 g, 11.5 mmol) in THF (30 mL). The resulting solution was stirred at −78° C. for 4 hours and then allowed to warm to room temperature overnight. The solvent was evaporated and the residue dissolved in water and extracted with ether. The combined organic phase was washed with brine, dried over magnesium sulfate, filtered and evaporated to give the title compound (4.71 g) which was used in the next step without purification. ¹H NMR (300 MHz, CDCl₃) δ7.25 (d, J=8.3 Hz, 2H), 6.82 (d, J=15.2 Hz, 2H), 4.54 (m, 1H), 3.76-3.79 (m, 8H), 3.68 (m, 1H), 2.12-2.38 (m, 7H), 1.55 (s, 3H), 1.42 (s, 9H), 1.28 (s, 3H). LRMS (APIMS) m/z 469 (MH⁺).

22b. tert-Butyl 3-(5-(1-((4-methoxyphenyl)methylthio)-isopropyl)-3,6-dioxopiperazin-2-yl) propanoate

The product of Example 22a (4.71 g) in toluene (60 mL) was refluxed for 24 hours, cooled to room temperature and stored at 4° C. overnight. The solid was filtered, triturated with ether, filtered and dried to give the title compound (0.85 g). ¹H NMR (300 MHz, CDCl₃) δ7.20 (d, J=8.5 Hz, 2H), 6.94 (br s, 2H), 6.81 (d, J=8.5 Hz, 2H), 4.18 (t, J=4.8 Hz, 1H), 3.86 (s, 3H, 3.77 (s, 2H), 3.75 (s, 1H), 2.36 (t, J=4.8 Hz, 2H), 2.11 (m, 2H), 1.62 (s, 3H), 1.50 (s, 9H), 1.33 (s, 3H). LRMS (APIMS) m/z 437 (MH⁺).

22c. 3-(5-(1-Methyl-1-sulfanylethyl)-3,6-dioxopiperizin-2-yl)propanoic acid

A solution of the product of Example 22b (0.85 g, 1.95 mmol), anisole (1 mL) and trifluoroacetic acid (0.5 mL) in dichloromethane (4 mL) was cooled to 0° C. and then treated dropwise with trifluoromethanesulfonic acid (0.97 mL). The resultant solution was stirred at 0° C. for 30 min and at room temperature for 1 hour, diluted with ether and water and the precipitate filtered. The solid was triturated twice, first with acetonitrile:ether (1:4) and then with methanol to give the title compound (0.35 g, 69%). ¹H NMR (300 MHz, DMSO-d6) δ8.20 (s, 1H), 7.98 (s, 1H), 4.02 (br s, 1H), 3.42 (s, 1H), 2.54 (s, 1H), 2.07 (m, 2H), 1.99 (m, 2H), 1.23 (s, 3H), 1.14 (s, 3H). ¹³C NMR (75 MHz, DMSO-d6) δ174.1, 168.1, 166.0, 64.8, 52.6, 49.6, 30.6, 29.8, 28.9, 26.5. LRMS (APIMS) m/z 261 (MH⁺). Anal. Calcd for C₁₀H₁₆N₂O₄S: C, 46.14; H, 6.21; N, 10.76. Found: C, 45.63; H, 6.08; N, 10.55.

22d. 3-(5-(1-Methyl-1-(nitrosothio)ethyl)-3,6-dioxopiperizin-2-yl)propanoic acid

To a solution of tert-butyl nitrite (38 μL, 294 mg, 0.29 mmol) in dichloromethane (1 mL) was added dropwise a solution of the product of Example 22c (50 mg, 0.19 mmol) in DMF (1 mL). The resultant solution was stirred for 25 minutes at room temperature in the dark and the residue after evaporation of the solvent triturated with dichloromethane to give the title compound (47 mg, 85%). ¹H NMR (300 MHz, CDCl₃) δ10.95 (br s, 1H), 8.78 (s, 1H), 8.27 (s, 1H), 4.31 (s, 1H), 3.74 (s, 1H), 1.97-2.27 (m, 10H). ¹³C NMR (75 MHz, CDCl₃) δ174.0, 167.7, 165.1, 63.5, 62.1, 52.3, 28.7, 26.7, 26.0. LRMS (APIMS) m/z 290 (MH⁺).

### EXAMPLE 23

2-(Acetylamino)-N-((2-(nitrosothio)adamantan-2-yl)methyl)acetaniide

23a. 2-sulfanyladamantane-2-carbonitrile

Adamantane-2-thione (3.5 g, 21 mmol) was dissolved in a mixture of THF (40 mL) and ethanol (40 mL) and then treated with sodium cyanide (3.1 g, 63 mmol). The reaction mixture was stirred at room temperature for 45 minutes. The volatile material was removed by evaporation and the residue was diluted with water, neutralized carefully with 2N HCl and extracted with ethyl acetate. The combined organic phase was dried over sodium sulfate, filtered and evaporated to give the title compound (4.25 g, 100%). ¹H NMR (300 MHz, CDCl₃) δ2.58 (s, 1H), 2.30 (d, 4H), 2.13 (m, 2H), 1.85-1.99 (m, 4H), 1.66-1.82 (m, 4H). ¹³C NMR (75 MHz, CDCl₃) δ122.9, 46.4, 37.6, 37.2, 35.2, 30.7, 26.6, 25.9. Anal. Calcd for C₁₁H₁₅NS: C, 68.35; H, 7.82; N, 7.24, Found: C, 68.43; H, 7.70; N, 7.15. LRMS (APIMS) m/z 211 (MNH₄⁺).

23b. 2-(Phenylmethylthio)adamantane-2-carbonitrile

A mixture of the product of Example 23a (3.7 g, 19.2 mmol), potassium carbonate (2.9 g, 21.1 mmol) and benzyl bromide (3.6 g, 21.1 mmol) in DMF (25 mL) was stirred at room temperature for 20 hours. The reaction mixture was diluted with a large volume of ethyl acetate, washed with water (×6), brine, dried over sodium sulfate, filtered and evaporated to give the title compound (4 g, 74%). Mp. 73-75° C. ¹H NMR (300 MHz, CDCl₃) δ7.25-7.50 (m, 5H), 4.05 (s, 2H), 1.59-2.38 (m, 14H). ¹³C NMR (75 MHz, CDCl₃) δ136.6, 129.2, 128.6, 127.4, 121.2, 51.9, 37.4, 35.4, 35.2, 34.9, 30.8, 26.6, 26.5. Anal. Calcd for C₁₈ H₂₁NS: C, 76.28; H, 7.47; N, 4.94, Found: C, 76.36; H, 7.49; N, 4.85. LRMS (APIMS) m/z 301 (MNH₄⁺).

23c. (2-(Phenylmethylthio)adamantan-2-yl)methylamine

To a solution of the product of Example 23b (6 g, 21.2 mmol) in THF (125 mL) was added, dropwise, a solution of lithium aluminum hydride (1M in THF, 42 mL, 42 mmol). After the addition was complete, the reaction mixture was stirred at room temperature for 10 minutes and then refluxed for 3 hours. The solution was cooled to room temperature and quenched carefully with cold, saturated, sodium bicarbonate solution and extracted with ethyl acetate. The combined organic phase was washed with brine, dried over sodium sulfate, filtered and evaporated. The residue was chromatographed (dichloromethane:methanol:triethylamine 95:4:1) to give the title compound (2.6 g, 43%). Mp. 82-85° C. ¹H NMR (300 MHz, CDCl₃) δ7.14-7.38 (m, 5H), 3.55 (s, 2H), 3.01 (s, 2H), 2.56 (d, 2H), 1.51-2.05 (m, 14H). ¹³C NMR (75 MHz, CDCl₃) δ138.2, 129.0, 128.4, 126.8, 60.9, 44.2, 39.1, 33.4, 32.8, 32.4, 30.7, 28.0, 27.4. Anal. Calcd for C₁₈H₂₅NS: C, 75.21; H, 8.77; N, 4.87, Found: C, 75.38; H, 8.83; N, 4.69. LRMS (APIMS) m/z 288 (MH⁺).

23d. 2-(Acetylamino)-N-((2-(phenylmethylthio)adamantan-2-yl)methyl)acetamide

To a mixture of the product of Example 23c (1.3 g, 4.5 mmol), 4-dimethylaminopyridine (0.28 g, 2.2 mmol) and N-acetylglycine (0.53 g, 4.5 mmol) in DMF (25 mL) was added 1-(3-(dimethylamino)propyl)-3-ethylcarbodiimide hydrochloride (0.95 g, 5 mmol). The reaction mixture was stirred at room temperature overnight, diluted with a large volume of ethyl acetate, washed with water (×8), brine and dried over sodium sulfate. Filtration and evaporation gave the title compound (1.6 g, 94%). Mp. 132-134° C. ^{H NMR (}300 MHz, CDCl₃) δ7.25-7.34 (m, 5H), 6.61 (br s, 1H), 6.50 (br s, 1H), 3.93 (d, J=5.1 Hz, 2H), 3.72 (d, J=5.4 Hz, 2H), 3.58 (s, 2H), 2.53 (d, J=12.1 Hz, 2H), 2.04 (s, 3H), 1.58-2.16 (m, 12H). ¹³C NMR (75 MHz, CDCl₃) δ170.3, 168.6, 137.8, 128.7, 127.1, 58.2, 43.1, 41.7, 38.9, 33.0, 32.9, 32.8, 30.7, 27.7, 27.1, 22.8. Anal. Calcd for C₂₂H₃₀N₂O₂S: C, 68.36; H, 7.82; N, 7.24, Found: C, 68.18; H, 7.98; N, 7.46. LRMS (APIMS) m/z 387 (MH⁺).

23e. 2-(Acetylamino)-N-((2-sulfanyladamantan-2-yl)methyl)acetamide

To a suspension of the product of Example 23d (1 g, 2.57 mmol) in liquid ammonia (10 mL) was added enough sodium to give a permanent blue colour (approx 200 mg). The reaction mixture was stirred for 20 min, quenched with ammonium chloride and the ammonia allowed to evaporate. The residue was suspended in ethyl acetate, washed with 2N HCl, brine and dried over sodium sulfate. Filtration and evaporation gave the title compound (600 mg, 78%). Mp. 154-157° C. ^{1H NMR (}300 MHz, CDCl₃) o 6.73 (br s, 1H), 6.48 (br s, 1H), 3.99 (d, J=5.1 Hz, 2H), 3.76 (d, J=5.9 Hz, 2H), 2.42 (d, J=12.6 Hz, 2H), 2.14 (d, J=13.1 Hz, 2H), 2.05 (s, 3H), 2.14 (d, J=13.1 Hz, 2H), 1.62-1.96 (m, 9H). ¹³C NMR (75 MHz, CDCl₃) δ170.6, 168.9, 57.9, 48.7, 43.4, 39.0, 36.3, 33.9, 33.2, 27.9, 26.8, 23.0. Anal. Calcd for C₁₅H₂₄N₂O₂S 1% H₂O: C, 60.17; H, 8.18; N, 9.35, Found: C, 59.92; H, 7.92; N, 9.38. LRMS (APIMS) m/z 297 (MH⁺).

23f. 2-(Acetylamino)-N-((2-(nitrosothio)adamantan-2-yl)methyl)acetamide

A solution of the product of Example 23e (0.5 g, 1.7 mmol) in a minimum amount of dichloromethane was added to a solution of tert-butyl nitrite (450 μL, 347 mg, 3.37 mmol) in dichloromethane (5 mL). The reaction mixture was stirred in the dark at room temperature for 40 minutes. The solvent was evaporated and the residue chromatographed (ethyl acetate:methanol 97:3) followed by recrystallization from ethyl acetate/ether to give the title compound (0.34 g, 62%). ¹H NMR (300 MHz, CDCl₃) δ6.85 (br s, 1H), 6.61 (br s, 1H), 4.55 (d, J=6.0 Hz, 2H), 3.86 (d, J=4.7 Hz, 2H), 2.46-2.63 (m, 4H), 1.97 (s, 3H), 1.71-2.12 (m, 10H). ¹³C NMR (CDCl₃) δ170.7, 169.3, 69.8, 45.1, 43.5, 38.8, 34.0, 33.8, 33.2, 27.5, 27.2, 22.7. Anal. Calcd for C₁₅H₂₃N₃O₃S: C, 55.36; H, 7.12; N, 12.91, Found: C, 55.66; H, 7.16; N LRMS (APIMS) m/z 326 (MH⁺).

### EXAMPLE 24

Adamantanylnitrosothio

24a. Adamantanylthiocarboxamidine hydrobromide

The was prepared as described by Khullar et al., (J. Org. Chem., 36: 3038-3040, 1971). A mixture of 1-bromoadamantane (10.7 g, 50 mmol) and thiourea (7.6 g, 100 mmol) in acetic acid (50 mL) and hydrobromic acid (48%, 25 mL) was refluxed for 3 hours. After standing at room temperature the solid formed was collected by filtration and recrystalised from ethanol to give the title compound (4.7 g, 32%). Mp 227-230° C. ¹H NMR (300 MHz, DMSO-d6) δ9.11 (br s, 4H), 1.89-2.05 (m, 9H), 1.50-1.65 (m, 6H). LRMS (APIMS) m/z 211 (MH⁺ for the free base).

24b. Adamantanethiol

A solution of the product of Example 24a (4.7 g, 16.2 mmol) in a mixture of ethanol (15 mL) and sodium hydroxide (5%, 45 mL) was stirred overnight at room temperature. The reaction mixture was diluted with water, acidified with concentrated HCl, extracted with ether and the organic phase washed with brine and dried over sodium sulfate. The residue after filtration and evaporation was chromatographed (neat hexane) to give the title compound (1.3 g, 48%). Mp 101-103° C. (lit (Khullar et al., J. Org. Chem., 36: 3038-3040, 1971)102-104°

24c. Adamantanylnitrosothio

A solution of the product of Example 24b (500 mg, 2.97 mmol) in dichloromethane (3 mL) was added, dropwise, rapidly to a solution of tert-butyl nitrite (792 μL, 613 mg, 6 mmol) in dichloromethane (5 mL). The resultant solution was stirred at room temperature in the dark for 40 minutes. The solvent was evaporated and the residue chromatographed twice (neat hexane) to give the title compound (230 mg, 39%). Mp. 58-60° C. (lit (Girard, P.; Guillot, N.; Motherwell, W. B.; Hay-Motherwell R. S.; Potier, P. Tetrahedron, 55: 3573-3584, 1999) 58-60° C.)

### EXAMPLE 25

(2-Methyladamantan-2-yl)nitrosothio

25a. Spiroadamantane-2,2′-thiirane

To sodium hydride (60% in mineral oil, 1.1 g, 27.5 mmol) in a mixture of DMSO (80 mL) and THF (20 mL) was added trimethylsulfoxonium iodide (5.8 g, 26.3 mmol) in one portion. The reaction mixture was stirred at room temperature for 15 minutes, then a solution of adamantane-2-thione (4.1 g, 24.5 mmol) in THF (50 mL) was added. The reaction mixture was stirred for an additional 30 minutes at room temperature and then at 90° C. for 1.5 hours. The solution was cooled to room temperature and then was quenched carefully with water (10 mL). Saturated sodium chloride (100 mL) was added, followed by water, to dissolve the solid. The mixture was extracted with hexane. The combined organic layers were washed with saturated sodium chloride, dried over sodium sulfate and filtered. The residue after evaporation was chromatographed (neat hexane) to give the title compound (4.1 g, 82%). Anal. Calcd for C₁₁H₁₆S: C, 73.27; H, 8.94; S, 17.78. Found: C, 73.18; H, 8.69; S, 17.89

25b. 2-Methyladamantane-2-thiol

A solution of the product of Example 25a (0.75 g, 4.15 mmol) in THF (20 mL) was treated with a solution of lithium aluminum hydride (1M in THF, 4 mL, 4 mmol). The reaction mixture was refluxed for 6 hours, cooled to room temperature, quenched with 2N HCl, diluted with water and then extracted with ether. The combined organic phase was washed with brine, dried over sodium sulfate, filtered and evaporated. The residue was chromatographed (neat hexane) to give the title compound (0.5 g, 66%). Mp. 154-155° C. Anal. Calcd for C₁₁H₁₈S: C 72.46; H, 9.95. Found: C, 72.41; H, 9.93.

25c. (2-Methyladamantan-2-yl)nitrosothio

A solution of the product of Example 25b (0.4 g, 2.18 mmol) was cooled to 0° C. and treated with tert-butyl nitrite (0.38 mL, 0.29 g, 2.88 mmol). The reaction mixture was stirred over ice for 1 hour and then at room temperature for 1 hour in the dark. The solvent was evaporated and the residue chromatographed on silica to give the title compound (0.43 g, 93%). Anal. Calcd for C₁₁H₁₇NOS: C, 62.52; H, 8.11; N, 6.63. Found: C, 62.70; H, 7.98; N, 6.45.

### EXAMPLE 26

Phenylmethyl 4-(hydroxymethyl)-4-(nitrosothio)piperidinecarboxylate

26a. 4-Piperidinylmethan-1-ol

To a solution of ethyl isonipecotate (20 g, 127 mmol) in dry ether (160 mL) was added, dropwise, a solution of lithium aluminum hydride (1M in tetrahydrofuran, 92 mL, 92 mmol) at 0° C. The resultant solution was stirred at 0° C. for 1 hour. The excess lithium aluminum hydride was destroyed carefully by addition of sodium sulfate decahydrate. The resulting granular white precipitate was filtered and washed with 10% methanol in dichloromethane. The filtrate was dried over sodium sulfate to give the title compound (10.1 g, 69%) ¹H NMR (300 MHz, CDCl₃) δ3.67 (s, 2H), 3.05 (br d, J=12.0 Hz, 2H), 2.51-2.60 (m, 2H), 1.68 (br d, J=13.1 Hz, 2H), 1.55-1.59 (m, 1H), 1.06-1.19 (m, 2H). LRMS (APIMS) m/z 116 (MH⁺).

26b. Phenylmethyl 4-(hydroxymethyl)piperidinecarboxylate

To a stirred solution of the product of Example 26a (4.69 g, 41.0 mmol) in dichloromethane (40 mL) was added, dropwise, benzyl chloroformate (5.81 mL, 6.95 g, 41.0 mmol) followed by diisopropylethylarnine (7.1 mL, 5.26 g, 41.0 mmol) at 0° C. The resultant mixture was stirred at room temperature for 18 hour, and then washed with water, 5% HCl, brine and dried over sodium sulfate. The residue was filtered, evaporated and then chromatographed (ethyl acetate:hexane 1:1) to give the title compound (4.52 g, 45%). ¹H NMR (300 MHz, CDCl₃) δ7.26-7.38 (m, 5H), 5.11 (s, 2H), 4.12-4.22 (m, 2H), 3.48 (d, J=6.1 Hz, 2H), 2.77 (br t, J=12.6 Hz, 2H), 1.59-1.74 (m, 3H), 1.09-1.25 (m, 2H). ¹³C NMR (75 MHz, CDCl₃) δ155.4, 137.0, 128.6, 128.0, 127.9, 67.5, 44.0, 38.8, 28.7, 14.3. LRMS (APIMS) m/z 250 (MH⁺).

26c. Phenylmethyl 4-formylpiperidinecarboxylate

To a stirred solution of oxalyl chloride (2M solution in dichloromethane, 10.9 mL, 21.9 mmol) was added DMSO (3.1 mL, 3.4 g, 43.8 mmol) in dichloromethane (6 mL) over a period of 15 minutes. The product of Example 26b (4.4 g, 17.5 mmol) in dichloromethane (7 mL) was then added at −78° C. over a period of 15 minutes. The resultant solution was stirred at −78° C. for 1 hour and then triethylamine (12.2 mL, 8.86 g, 87.5 mmol) was added, dropwise, over a period of 15minutes. The mixture was further stirred at −78° C. for 30 min and then at 0° C. for 15 min. The reaction mixture was quenched with water and extracted with dichloromethane. The combined organic phase was washed with 1% HCl, water, dried over sodium sulfate, filtered and evaporated to give the title compound (4.4 g, 100%) which was used in the next step without purification. ¹H NMR (300 MHz, CDCl₃) δ9.65 (s, 1H), 7.28-7.38 (m, 5H), 5.12 (s, 2H), 4.04 (br d, J=13.1 Hz, 2H), 2.97-3.06 (m, 2H), 2.38-2.45 (m, 1H), 1.88-1.92 (m,2H), 1.52-1.64 (m, 2H). ¹³C NMR (75 MHz, CDCl₃) δ202.7, 155.2, 136.7, 128.5, 128.6, 127.9, 67.2, 47.8, 43.0, 25.1. LRMS (APIMS) m/z 248 (MH⁺).

26d. Phenylmethyl 4-(hydroxymethyl)-4-sulfanylpiperidinecarboxylate

To a stirred solution of the product of Example 26c (4.4 g, 17.8 mmol) in carbon tetrachloride (8 mL) was added, dropwise, sulfur monochloride (0.85 mL, 1.4 g, 10.7 mmol) over a period of 5 min at 50° C. After a short lag phase (10-15 min), evolution of HCl gas was observed. After the gas evolution had ceased, the mixture was stirred at 55° C. for 0.5 hours and then cooled to room temperature. The residue, after evaporation of the solvent, was chromatographed (ethyl acetate:hexane 1:2 to 1:1) to give the product (4.28 g, 86%) which was used in the next step without purification. ¹H NMR shows significant line broadening, possibly due to rotomer formation. LRMS (APIMS) m/z 557 (MH⁺). To a stirred solution of this disulfide (0.5 g, 0.90 mmol) in THF(13 mL) was added dropwise lithium aluminum hydride (1 M solution in THF, 1.8 mL, 1.8 mmol) at 0° C. under nitrogen. The resulting solution was stirred at room temperature for 30 minutes. The excess lithium aluminum hydride was destroyed carefully by the addition of sodium sulfate decahydrate and the resulting granular precipitate was filtered and washed with ethyl acetate. The filtrate was dried over sodium sulfate and evaporated. The residue was chromatographed (ethyl acetate:hexane 1:1) to give the title compound (201 mg, 40%). ¹H NMR (300 MHz, CDCl₃) δ7.29-7.39 (m, 5H), 5.12 (s, 2H), 3.92-4.10 (m, 2H), 3.49 (s, 2H), 3.20-3.38 (m, 2H), 2.27 (br s, 1H), 1.55-1.75 (m, 4H), 1.36 (s, 1H). ¹³C NMR (75 MHz, CDCl₃) δ155.2, 136.7, 128.6, 128.1, 127.9, 73.3, 67.2, 50.4, 40.2, 35.0. Anal. Calcd for C₁₄H₁₉NO₃S: C, 59.76; H, 6.81; N, 4.98. Found: C, 59.65; H, 6.74; 4.82. LRMS (EI) m/z 282 (MH⁺), 304 (MNa⁺).

26e. Phenylmethyl 4-(hydroxymethyl)-4-(nitrosothio)piperidinecarboxylate

A solution of the product of Example 26d (0.1 g, 0.36 mmol) in dichloromethane (1 mL) was added, dropwise, to a solution of tert-butyl nitrite (0.77 μL, 0.60 mg, 0.58 mmol) in dichloromethane (1 mL). The resulting solution was stirred at 0° C. for 20 min and then at room temperature for 10 min in the dark. The residue after evaporation of the solvent was chromatographed (ethyl acetate:hexane 1:2) to give the title compound (72 mg, 65%). ¹H NMR (300 MHz, CDCl₃) δ7.29-7.37 (m, 5H), 5.15 (s, 2H), 4.24 (s, 2H), 4.11-4.15 (m, 2H), 3.13-3.21 (m, 2H), 2.48-2.53 (m, 2H), 2.23-2.38 (m, 2H). LRMS (EI) m/z 311 (MH⁺).

### EXAMPLE27

4-Methyl-4-(N-(2-methyl-2-(nitrosothio)propyl)carbamoyl)pentanoic acid

27a. 4-Methyl-4-(N-(2-methyl-2-(sulfanylpropyl)carbamoyl)pentanoic acid

To a solution of 1-amino-2-methyl-2-propanethiol hydrochloride (1.25 g, 8.82 mmol) in dicloromethane (20 mL) at 0° C. was added triethylamine (1.07 g, 10.6 mmol) followed immediately by the addition of α,α-dimethylglutaric anhydride(1.14 g, 8.02 mmol). The resulting mixture was stirred at 0° C. for 1 hour and then overnight at ambient temperature. The reaction mixture was diluted with methylene chloride, washed with H₂O, 10% HCl, and brine. The combined aqueous layers were extracted ethyl acetate (2×). The combined organic extracts were dried over sodium sulfate, filtered and the solvent removed in vacuo to give the title compound (1.88 g, 95%) as a white solid. Mp 107-110° C.; 1H NMR (CDCl₃) δ10.93 (bs, 1H), 6.24 (bs, 1H), 3.32 (d, J=6.2 Hz, 2H), 2.28 (m, 2H), 1.93 (m, 2H), 1.63 (s, 1H), 1.35 (s, 6H), 1.23 (s, 6H); LRMS (APIMS) m/z 248 (MH⁺).

27b. 4-Methyl-4-(N-(2-methyl-2-(nitrosothio)propyl)carbamoyl)pentanoic acid

To the product of Example 27a (1.87 g, 7.56 mmol) in methylene chloride (20 mL) at ambient temperature was added tert-butyl nitrite (819 mg, 7.94 mmol) under argon and the reaction mixture was stirred at ambient temperature for 1hour. The solvent was removed in vacuo to give the title compound (2.06 g, 99%) as a dark green solid. Mp 93-96° C.; ¹H NMR (CDCl₃) δ10.21 (vbs, 1H), 6.15 (bs, 1H), 4.01 (d, J=6.4Hz, 2H), 2.22 (m, 2H), 1.88 (m, 2H), 1.86 (s, 6H), 1.21 (s, 6H); LRMS (APIMS) m/z 277 (MH⁺).

### EXAMPLE 28

N,N-Dimethyl-2-(2-(nitrosothio)adamantan-2-yl)acetamide

28a. Spiro(adamantane-2,4′-thietane)-12-one

A mixture of the product of Example 12b (516 mg, 2.28 mmol) and 1-(3-(dimethylamino) propyl)-3-ethylcarbodiimide hydrochloride (445 mg, 2.32 mmol) in dichloromethane (10 mL) was stirred at room temperature for 1 hour, diluted with dichloromethane and washed with 0.1 M HCl and brine. The organic phase was dried over magnesium sulfate, filtered, evaporated and chromatographed (ethyl acetate:hexane: 1:3, then 1:1) to give the title compound (0.41 g, 86%). ¹H NMR (300 MHz, CDCl₃) δ3.61 (s, 2 H), 2.20 (m, 2 H), 1.78-1.95 (m, 12 H). ¹³C NMR (75 MHz, CDCl₃) δ191.8, 63.4, 54.9, 39.9, 36.5 35.6, 33.7, 26.6, 25.8. LRMS (APIMS) m/z 209 (M+H⁺), 226 (MNH₄⁺).

28b. N,N-Dimethyl-2-(2-sulfanyladamantan-2-yl)acetamide

To the product of Example 28a (1.35 g, 6.5 mmol) in dichloromethane (15 mL) at room temperature was added dimethylamine (2.0 M in methanol, 5.5 mL, 11 mmol). The reaction mixture was stirred at room temperature for 40 minutes, evaporated to dryness and the residue chromatographed (neat dichloromethane) to give the title compound (1.30 g, 79%). ¹H NMR (300 MHz, CDCl₃) δ3.09 (s, 2H), 3.00 (s, 3H), 2.97 (s, 3H), 2.53-2.57 (m, 2H), 2.16 (m, 2H), 2.07-2.11 (m, 2H), 1.86 (m, 2H), 1.70-1.76 (m, 4H), 1.60-1.65 (m, 2H). ¹³C NMR (75 MHz, CDCl₃) δ171.2, 54.6, 42.8, 39.2, 38.0, 37.8, 35.4, 33.8, 33.4, 27.7, 27.0. LRMS (APIMS) m/z 254 (MH⁺).

28c. N,N-Dimethyl-2-(2-(nitrosothio)adamantan-2-yl)acetamide

To the product of Example 28b (450 mg, 1.77 mmol) in dichloromethane (5 mL) was added tert-butyl nitrite (430 μL, 373 mg, 3.62 mmol) at room temperature. The reaction mixture was stirred at room temperature for 20 min, evaporated to dryness, and treated with dichloromethane and water. The organic phase was separated, dried with magnesium sulfate, filtered and evaporated. The residue was chromatographed (neat dichloromethane) to give the title compound (399 mg, 80%). ¹H NMR (300 MHz, CDCl₃) δ3.73 (s, 2H), 3.02 (s, 2H), 2.82 (s, 6H), 2.41-2.45 (m, 2H), 2.08-2.13 (m, 3H), 1.92-1.96 (m, 3H), 1.86 (m, 2H), 1.70-1.77 (m, 2H). ¹³C NMR (75 MHz, CDCl₃) δ170.1, 67.4, 39.4, 38.9, 37.8, 35.6, 35.3, 33.9, 33.4, 27.32, 27.28. LRMS (APIMS) m/z 283 (MH⁺).

### EXAMPLE 29

tert-Butyl 2-(2-(nitrosothio)adamantan-2-yl)acetate

29. tert-Butyl 2-(2-(nitrosothio)adamantan-2-yl)acetate tert-butyl nitrite (0.5 mL, 3.78 mmol) was added to an ice-cold solution of the product of Example 12a (0.825 g, 2.92 mmol) in dichloromethane (15 mL). The solution was stirred in the dark in an ice-bath for 30 minutes and then at room temperature for 2 hours. The volatiles were evaporated and the residue chromatographed (ethyl acetate:hexane 1:20) to give the title compound (0.87 g, 96%). Mp 85-87° C. ¹H NMR (300 MHz, CDCl₃) δ3.61 (s, 2H), 2.76 (m, 2H), 1.60-2.60 (m, 12H), 1.31 (s, 9H). ¹³C NMR (75 MHz, CDCl₃) δ169.8, 80.8, 66.1, 43,4, 38.9, 35.6, 33.8, 33.1, 27.9, 27.2. LRMS (EI) m/z 312 (MH⁺).

### EXAMPLE 30

1,1-Dimethyl-2-(4-(2-pyridyl)piperazinyl)ethyl)nitrosothiol

30a. 2-Methyl-1-(4-(2-pyridyl)piperazinyl)propane-2-thiol

A stirred solvent-free mixture of 1-(2-pyridyl)piperazine (1.60 g, 9.8 mmol) and 2,2-dimethylthiirane (1.06 g, 12 mmol) was heated at 80° C. for 2 hours. The volatile was removed by evaporation, and the resulting material was purified by crystallization from 1:1 EtOAc/hexanes to give the title compound (1.8 g, yield 73%) as white needles. Mp 108-110° C.

¹H NMR (300 MHz, CDCl₃) δ8.18-8.16 (m, 1H), 7.54-7.34 (m, 1H), 6.67-6.56 (m, 2H), 3.52 (t, J=5.0 Hz, 4H), 2.74 (t, J=5.0 Hz, 4H), 2.43 (s, 2H), 1.32 (s, 6H). ¹³C NMR (75 MHz, CDCl₃) δ159.3, 147.7, 137.1, 112.9, 106.8, 71.1, 55.0, 46.0, 45.3, 30.1. LRMS (API-TIS) m/z 252 (MH⁺).

30b. (1,1-Dimethyl-2-(4-(2-pyridyl)piperazinyl)ethyl)nitrosothiol

To a stirred solution of the product of Example 30a (1.50 g, 5.98 mmol) in MeOH (50 mL) was added concentrated hydrochloric acid (12N, 1.54 mL, 18 mmol). After 5 minutes, tert-butyl nitrite (90% tech, 0.924 mL, 7 mmol) was added dropwise. The reaction mixture was stirred at room temperature for 10 minutes, diluted with EtOAc, washed with 2 M sodium carbonate twice. The organic layer were dried over anhydrous sodium sulfate filtered, and concentrated. The crude product was purified by chromatography (silica gel, 1:5 EtOAc/hexanes) to give the title compound (1.22 g, yield 71%) as a green solid. Mp 96° C. ¹H NMR (300 MHz, CDCl₃) δ8.17 (dd, J=4.7, 0.95 Hz, 1H), 7.45-7.41 (m, 1H), 6.62-6.57 (m, 2H), 3.49 (t, J=4.9 Hz, 4H), 3.04 (s, 2H), 2.73 (t, J=5.1 Hz, 4H), 1.91 (s, 6H). ¹³C NMR (75 MHz, CDCl₃) δ159.4, 147.8, 137.3, 113.2, 106.9, 68.2, 58.7, 55.1, 45.4, 27.0. LRMS (API-TIS) m/z 281 (MH⁺).

### EXAMPLE 31

2-(2-(Nitrosothio)adamantan-2-yl)ethyl 4-methoxybenzoate

31. 2-(2-(Nitrosothio)adamantan-2-yl)ethyl 4-methoxybenzoate

Dicyclohexylcarbodiirnide (0.68 g, 3.3 mmol) in dichloromethane (5 mL) was added dropwise to a stirred solution of the product of Example 27b (0.79 g, 3.3 mmol), 4-methoxybezoic acid (0.5 g, 3.3 mmol) and 4-dimethylaminopyridine (0.4 g, 3.3 mmol) in dimethylformamide (6 mL) at room temperature. The resulting green solution was stirred at room temperature for 2 hours in the dark. The precipitate was filtered and washed with dichloromethane (25 mL). The filtrate was washed with water and dried over anhydrous sodium sulfate. The residue after evaporation of the solvent was chromatographed on silica gel eluting with 1:4 ethyl acetate:hexane to give the title compound (0.5 g, 73% based on recovered 27b) and 1:1 ethyl acetate:hexane to give unreacted 27b (0.35 g). Mp 100-101° C. ¹H NMR (300 MHz, CDCl₃) δ7.95 (d, J=8.5 Hz, 2H), 6.92 (d, J=8.8 Hz, 2H), 4.49 (t, J=7.2 Hz, 2H), 3.87 (s, 3H), 3.21 (t, J=7.1 Hz, 2H), 2.38-2.62 (m, 4H), 1.64-2.18 (m, 10H). ¹³C NMR (75 MHz, CDCl₃) δ166.5, 163.6, 131.7, 122.8, 113.8, 68.2, 61.6, 55.6, 39.1, 36.1, 35.8, 34.1, 33.4, 27.5, 27.4. mass spectrum (API-TIS) m/z 393 (MNH₄⁺). Anal. Calcd for C₂₀H₂₅NO₄S: C, 63.98; H, 6.71; N, 3.73; S, 8.54. Found: C, 64.04; H, 6.77; N, 3.47; S, 8.82.

### EXAMPLE 32

(1,1-Dimethyl-2-(2-1,2,3,4-tetrahydroisoquinolyl)ethyl)nitrosothio

32a. 2,2-Dimethylthiirane

A mixture of 2,2-dimethyloxirane (25 g, 346 mmol), water (50 ml), and potassium thiocyanate (67 g, 692 mmol) was stirred at room temperature for 20 hours. The residue after evaporation of the solvent was dissolved in dichloromethane, dried over anhydrous Na₂SO₄ and filtered. The filtrate was evaporated in vacuo to give the title compound (26.4 g, 87%). ¹H NMR (300 MHz, CDCl₃) δ2.41(s, 2 H), 1.62 (s, 6 H).

32b. 2-Methyl-1-(2-1,2,3,4-tetrahydroisoquinolyl)propane-2-thiol

A mixture of neat 1,2,3,4-tetrahydroisoquinoline (2 g, 15 mmol) and the product of Example 32a (1.5 g, 17 mmol) was heated at 80° C. for 4 hours. The reaction mixture was cooled to room temperature, poured into water and extracted with dichloromethane. The combined extracts were dried over anhydrous sodium sulfate and filtered. The volatiles were evaporated to give the title compound (1.3 g, 16%) as a white solid. ¹H NMR (300 MHz, CDCl₃) δ6.97-7.25 (m, 4H), 3.90 (s, 2H), 2.90-3.08 (m, 4H), 2.63 (s, 2H), 2.29 (s, 1H), 1.39 (s, 6H). ¹³C NMR (75 MHz, CDCl₃) δ135.7, 134.6, 128.8, 126.6, 126.2, 125.7, 71.3, 58.2, 53.4, 46.5, 30.3, 29.5. mass spectrum (API-TIS) m/z 222 (MH⁺).

32c. (1,1-Dimethyl-2-(2-1,2,3,4-tetrahydroisoquinolyl)ethyl)nitrosothio

A solution of the product of Example 32b (1.32 g, 5.97 mmol) and trifluoroacetic acid (0.92 mL, 1.36 g, 11.94 mmol) in dichloromethane (8 mL) was added dropwise to a solution of tert-butyl nitrite (1.17 mL of 90% solution, 0.92 g, 8.95 mmol) in dichloromethane (4 mL) at 0° C. The resulting solution was stirred for 30 minutes at 0° C. in the dark. The residue after evaporation of the solvent was chromatographed on silica gel eluting with 5:95 ethyl acetate:hexane to give the title compound (0.66 g, 44%) as a green oil. ¹H NMR (300 MHz, CDCl₃) δ6.94-7.20 (m, 4H), 3.86 (s, 2H), 3.22 (s, 2H), 2.82-2.98 (m, 4H), 1.94 (s, 6H), ¹³C NMR (75 MHz, CDCl₃) δ135.2, 134.5, 128.9, 126.7, 126.6, 125.7, 71.3, 68.2, 59.0, 58.2, 53.4, 29.3, 27.1. mass spectrum (API-TIS) m/z 251 (MH⁺), 221 (M-NO).

### EXAMPLE 33

4-(N-(((Nitrosothiocyclohexyl)methyl)carbamoyl)butanoic acid

33a. 1-Mercaptocyclohexane-1-carboxaldehyde disulphide

This compound was prepared from cyclohexanecarboxaldehyde and sulfur monochloride as described by Hayashi, K. et al., Macromolecules, 3: 5-9 (1970).

33b. Di((1Z)-2-aza-2-methoxyvinyl)cyclohexyl disulfide

A solution of 15 N NaOH (22 mL) was added to a stirred solution of the product of Example 33a (30 g, 0.1 mol) and methoxyamine hydrochloride (21.9 g, 0.26 mol) in absolute ethanol (600 mL) at room temperature. The resultant white suspension was heated at 80° C. for 3.5 hours and cooled to room temperature. The mixture was concentrated in vacuo and water (250 mL) was added. The aqueous layer was extracted with ethyl acetate, the combined organic phase was dried over anhydrous sodium sulfate, filtered, and concentrated in vacuo to give the title compound (34 g, 94%) as an oil. ¹H NMR (300 MHz, CDCl₃) δ7.15 (s, 2H), 3.92 (s, 6H), 1.90-2.07 (m, 4H), 1.62-1.80 (m, 8H), 1.32-1.62 (m, 8H). ¹³C NMR (75 MHz, CDCl₃) δ152.7, 61.9, 54.0, 34.4, 25.7, 22.9. mass spectrum (API-TIS) m/z 345 (MH⁺). Anal. Calcd for C₁₆H₂₈N₂O₂S₂: C, 55.78; H, 8.19; N, 8.13. Found: C, 56.06; H, 8.27; N, 7.85

33c. 1-Mercaptocyclohexane-1-methylamine

To a stirred solution of the product of the Example 33b (11.5 g, 33.4 mmol) in THF (60 mL) was added dropwise a solution of lithium aluminum hydride (66.7 mL of 1M in THF, 66.7 mmol) over a period of 20 minutes at room temperature under nitrogen. After the addition was complete the solution was stirred for 1 hour at room temperature and then at 60° C. for 16 hours. The excess lithium aluminum hydride was destroyed carefully by addition of sodium sulfate decahydrate. The granular white precipitate was filtered and washed with 10% methanol in dichloromethane. The filtrate was dried over anhydrous sodium sulfate, filtered and concentrated in vacuo to give the title compound as a viscous oil (7.0 g, 72%). ¹H NMR (CDCl₃) δ2.63 (s, 2H), 1.12-1.80 (m, 10H). ¹³C NMR (CDCl₃) δ155.6, 52.0, 37.1, 26.0,

33d. 4-(N-((Sulfanylcyclohexyl)methyl)carbamoyl)butanoic acid

Glutaric anhydride (5.19 g, 45.5 mmol) in dichloromethane (20 mL) was added dropwise to a solution of the product of Example 33c (6.6 g, 45.5 mmol) in dichloromethane (20 mL) at 0° C. The mixture was stirred at 0° C. for 1 hour. To this mixture, triethylamine (0.2 mL) was added. The stirring-was continued for further 30 minutes. The reaction mixture was diluted with dichloromethane, washed with 10% HCl, dried over anhydrous sodium sulfate, filtered and evaporated to give the title compound (10.5 g, 90%) as a white solid. Mp 73-74° C. ¹H NMR (300 MHz, d⁶-DMSO) δ12.00 (bs, 1H), 7.84 (bs, 1H), 3.26 (d, J=6.3 Hz, 2H), 2.36 (s, 1), 2.08-2.25 (m, 4H), 1.63-1.79 (m, 2H), 1.35-1.63 (m, 10H). ¹³C NMR (75 MHz, d⁶-DMSO) δ174.2, 172.1, 51.1, 50.2, 36.6, 34.4, 33.1, 25.4, 21.8, 20.8. mass spectrum (API-TIS) m/z 260 (MH⁺).

33e. 4-(N-(((Nitrosothiocyclohexyl)methyl)carbamoyl)butanoic acid

A solution of the product of Example 33d (8.99 g, 34.7 mmol) in dichloromethane (70 mL) was added dropwise to a solution of tert-butyl nitrite (6.2 mL, 5.3 mg, 52.1 mmol) in dichloromethane (9 mL) at 0° C. The resulting solution was stirred at 0° C. for 15 minutes and at room temperature for 15 minutes. The green precipitate was filtered and washed with hexane and dried under vacuo to give the title compound (8.0 g, 80%). Mp 108-110° C. ¹H NMR (300 MHz, d6-DMSO) δ11.07 (s, 1H), 8.05 (bt, 1H), 3.91 (d, J=6.3 Hz, 2H), 2.28-2.44 (m, 2H), 1.97-2.20 (m, 6H), 1.57-1.78 (m, 4H), 1.32-1.51 (m, 4H). ¹³C NMR (75 MHz, d⁶-DMSO) δ174.1, 172.1, 63.3, 47.7, 34.3, 33.6, 33.0, 24.9, 21.7, 20.7. mass spectrum (API-TIS) m/z 289 (MH⁺), 259 (M-NO). Anal. Calcd for C₁₂H₂₀N₂O₄S: C, 49.98; H, 6.99; N, 9.71; S, 11.12. Found: C, 50.15; H, 7.06; N, 9.54; S, 11.06.

### EXAMPLE 34

N-(2-Hydroxyethyl)-2-(2-(nitrosthio)adamantan-2-yl)acetamide

34a. 2-(2-Acetylthioadamant-2-yl)acetic acid

To the product of Example 12a (2.06 g, 7.3 mmol) in pyridine (11 mL) was added 4-dimethylaminopyridine (6 mg, 0.05 mmol) and acetic anhydride (6 mL, 6.49 g, 63.6 mmol). The resultant solution was stirred at room temperature overnight, concentrated to dryness and azeotroped three times with toluene to give an oil. To the oil was added dichloromethane (5 mL) and then trifluoroacetic acid (5 mL). After 30 minutes the reaction mixture was concentrated to dryness and azeotroped with dichloromethane three times to give a light yellow solid. The solid was triturated with dichloromethane and washed with dichloromethane to give the title compound (1.64 g, 83%). ¹H NMR (300 MHz, CDCl₃) δ9.4 (broad, 1H), 3.40 (s, 2H), 2.46 (m, 2H), 2.4-2.2 (m, 2H), 2.26 (s, 3H), 2.11-2.07 (m, 2H), 1.88 (m, 2H), 1.73-1.63 (m, 6H). ¹³C NMR (75 MHz, CDCl₃) δ196.4, 177.3, 60.9, 38.89, 38.86, 33.7, 32.8, 32.8, 27.14, 27.00. LRMS (APIMS) fm/z 269 (MH⁺).

34b. 2-(2-Acetylthioadamantan-2-yl)-N-(2-hydroxyethyl)acetamide

To the product of Example 34a (1.99 g, 7.4 mmol) in chloroform (10 mL) was added oxalyl chloride (1.0 mL, 1.45 g, 11.5 mmol) and N,N-dimethylformamide (25 μL). The solution was stirred at room temperature for 1 hour, concentrated to dryness then disolved in chloroform (9.4 mL). One half of this solution (4.7 mL) was slowly added to a solution of ethanolamine (260 μL, 263 mg, 4.3 mmol) and triethylamine (620 μL, 450 mg, 4.4 mmol) in chloroform (18 mL) at −78° C. The solution was stirred at room temperature for 30 minutes and washed with water and brine. The organic phase was dried over sodium sulfate, filtered and concentrated to dryness. The product was chromatographed (ethyl acetate) to give the title compound (1.0579 g, 92%). ¹H NMR (300 MHz, CDCl₃) δ6.50 (t, J=5.5 Hz, 1H), 3.97 (t, J=5.1 Hz, 1H), 3.64 (q, J=5.0 Hz, 2H), 3.35 (q, J=5.3 Hz, 2H), 3.21 (s, 2H), 2.45 (m, 2H), 2.33 (m, 5H), 2.14 (m, 2H), 1.87 (m, 2H), 1.7-1.6 (m, 6H). ¹³C NMR (75 MHz, CDCl₃) δ197.9, 171.6, 62.2, 61.3, 41.7 40.3, 38.6, 33.8, 33.4, 32.4, 31.5, 26.77, 26.70. LRMS (APIMS) m/z 312 (MH⁺).

34c. N-(2-Hydroxyethyl)-2-(2-sulfanyladamantan-2-yl)acetamide

The product of Example 34b (424 mg, 1.36 mmol) in methanol at 0° C. was saturated with ammonia. The reaction solution was stirred at room temperature for 1.5 hour and concentrated to dryness. The product was chromatographed (ethyl acetate:hexane 1:1 then ethyl acetate) to give the title compound (355 mg, 97%). ¹H NMR (300 MHz, CD₃OD) δ6.49 (br s, 1H), 3.75 (m, 2H), 3.45 (dd, J=4.6 Hz, 5.5 Hz, 2H), 2.87 (s, 2H), 2.68 (br s, 1H), 2.50 (m, 2H), 2.16 (m, 3H), 1.60-1.90 (m, 10H). ¹³C NMR (75 MHz, CD₃OD) δ174.0, 61.6, 55.1, 48.1, 42.8, 40.2, 39.6, 35.0, 34.3, 29.1, 28.5. LRMS (APIMS) m/z 270 (MH⁺).

34d. N-(2-Hydroxyethyl)-2-(2-(nitrosthio)adamantan-2-yl)acetamide

To the product of Example 34c (90.4 mg, 0.34 mmol) in acetic acid (1 mL) at 4° C. was added sodium nitrite (27.2 mg, 0.4 mmol). The reaction mixture was stirred at room temperature for 20 minutes, concentrated to dryness and azeotroped with toluene twice. The residue was treated with acetonitrile and chloroform and the solid was removed by filtration. The filtrate was concentrated and chromatographed (C18 gel, Water's Sep-Pak Vac 12cc (2 g) C18 Cartridges, WAT036915, acetonitrile:water 1:1) to give the title compound (68 mg, 68%). ¹H NMR (300 MHz, CDCl₃) δ5.74 (broad, 1H), 3.60 (m, 4H), 3.27 (m, 2H), 2.82 (s, 2H), 2.47 (m, 2H), 2.1-1.6 (m, 11H). ¹³C NMR (75 MHz, CDCl₃) δ171.0, 67.2, 62.2, 44.1, 42.2, 38.8, 35.8, 33.8, 33.2, 27.12, 27.07. LRMS (APIMS) m/z 299 (MH⁺).

### EXAMPLE 35

N-(2-(2-(Nitrosothio)adamantan-2-yl)ethyl)acetamide

35a. 2-(2-Aminoethyl)adamantane-2-thiol hydrochloride

The product of Example 12c (123.1 mg, 0.5463 mmol) in tetrahydrofuran (4.0 mL) was heated to reflux. Borane-methyl sulfide complex (2.0 M in tetrahydrofuran, 1.3 mL, 2.6 mmol) was slowly added. The mixture was refluxed for 1 hour, cooled to room temperature. Methanol was added to consume the excess borane-methyl sulfide. Anhydrous hydrochloric acid in ethyl ether was added and the resulting precipitate was collected by filtration, washed with tetrahydrofuran, and dried to give the title compound (75.3 mg, 56%). ¹H NMR (300 MHz, ⁻ CD₃OD) δ4.32 (br s, 3H), 2.30-2.94 (m, 2H), 2.29-2.25 (m, 2H), 2.05-2.00 (m, 2H), 1.93-1.89 (m, 2H), 1.54-1.44 (m, 10H). ¹³C NMR (75 MHz, CD₃OD) δ55.5, 40.0, 39.8, 39.2, 37.2, 34.9, 34.0, 29.2, 28.2. LRMS (APIMS) m/z 212 (MH⁺).

35b. 2-(2-(Nitrosothio)adamantan-2-yl)ethylamine hydrochloride

To the product of Example 35a (17.6 mg, 0.071 mmol) in N,N-dimethylformamide (0.4 mL) was added tert-butyl nitrite (11μL, 9.5 mg, 0.093 mmol). The reaction mixture was stirred at room temperature for 20 minutes, and then dried in vacuum to give the title compound (19.6 mg, 100%). ¹H NMR (300 MHz, CD₃OD) δ3.14 (m, 2H), 2.54-2.48 (m, 4H), 2.12-1.80 (m, 12H). ¹³C NMR (75 MHz, CD₃OD) δ68.3, 39.8, 36.6, 36.5, 36.1, 34.7, 33.9, 28.8, 28.6. LRMS (APIMS) m/z 241 (MH⁺).

35c. N-(2-(2-(Nitrosothio)adamantan-2-yl)ethyl)acetamide

To the product of Example of 35b (19.6 mg, 0.0708 mmol) in N,N-dimethylformamide (0.2 mL) was added triethylamine (20 μL, 14.5 mg, 0.143 mmol) and acetic anhydride (6.8 μL, 7.4 mg, 0.072 mmol). The reaction mixture was stirred at room temperature for 20 minutes, and dried in vacuum. The resultant product was treated with water and ethyl acetate. The organic phase was washed with brine and dried over magnesium sulfate. The product was chormatogrphed (ethyl acetate: hexane 1:1) to give the title compound (8.9 mg, 45%). ¹H NMR (300 MHz, CDCl₃) δ5.5 (br s, 1H), 3.40-3.34 (m, 2H), 2.95-2.89 (m, 2H), 2.54 (m, 2H), 2.44 (m, 2H), 2.08-1.72 (m, 10H), 1.94 (s, 3H). ¹³C NMR (75 MHz, CDCl₃) 5 170.1, 68.4, 38.9, 36.9 35.4, 35.2, 33.9, 33.0, 27.4, 27.1, 23.2. LRMS (APIMS) m/z 283 (MH⁺), 300 (MNH₄⁺).

### EXAMPLE 36

(3-Methylquinudidin-3-yl)nitrosothio hydrochlroide

36a. Spiro(oxirane-3,3′-quinudidine)

Quinudidin-3-one hydrochloride (15.07 g, 93.25 mmol) in water was neutralized with an aqueous solution of sodium hydroxide (4.47 g, 111.7 mmol) and the aqueous solution extracted with dichloromethane (4×). The combined organic extracts were dried over magnesium sulfate, filtered, and concentrated to give quinudidin-3-one (11.21 g, 89.56 mmol, 96%). To sodium hydride (2.26 g, 94.2 mmol) under nitrogen was added dimethyl sulfoxide (85 mL). The resultant mixture was stirred for 1 minute. Trimethylsulfoxonium iodide (20.79 g, 94.47 mmol) was added in portions under a stream of nitrogen. The resultant mixture was stirred at room temperature for 40 minutes. Then the quinudidin-3-one prepared above(11.21 g, 89.56 mmol) in tetrahydrofuran-dimethyl sulfoxide (20 mL-8 mL) was slowly added. The resultant mixture was stirred at room temperature for 15 minutes and at 57° C. for 40 minutes and then poured into water (450 mL). The aqueous solution was extracted with ethyl ether (4×) and with dichloromethane (8×). The combined organic extracts were dried over magnesium sulfate, concentrated to give an oil (13.89 g). The oil was distilled twice to give the title compound (b.p.=57° C., 0.15 Torr, 4.23 g, 34%). ¹H NMR (300 MHz, CDCl₃) δ3.12-3.08 (m, 1H), 3.07-2.82 (m, 5H), 2.75-2.69 (m, 2H), 2.05-1.86 (m, 1H), 1.86-1.63 (m, 2H), 1.63-1.42 (m, 1H),1.42-1.28 (m, 1H). ¹³C NMR (75 MHz, CDCl₃) δ59.5, 55.4, 53.2, 46.9, 46.6, 29.1, 24.8, 22.5. LRMS (APIMS) m/z 140 (MH⁺).

36b. 3-Methylquinudidine-3-thiol hydrochloride

Potassium thiocyanate (26.28 g, 270 mmol) was dissolved in water (19.6 mL) to give a 7.7 M solution. The product of Example 36a (1.7963 g, 12.904 mmol) was dissolved in the potassium thiocyanate solution (19.3 mL, 149 mmol). The reaction mixture was stirred for exact 135 minutes and dichloromethane (200 mL) was added. The aqueous solution was separated and extract with dichloromethane (50 mL). The combined dichloromethane extracts were washed with water (10 mL), dried over magnesium sulfate and concentrated to which mostly spiro(quinudidine-3,3′-thiirane) and some unreacted product of Example 36a (837 mg, total product). The entire work-up was completed within 15 minutes and immediately the product was dissolved in tetrahydrofuran (18 mL) and then lithium aluminum hydride (1.0 M, 9.0 mL) was added rapidly. The reaction mixture was stirred at room temperature for 20 minutes. Water was added and stirring continued for an additional 5 minutes. Dichloromethane was added. The organic phase was separated, dried over magnesium sulfate, filtered and concentrated. The product was immediately chromatographed (methanol:dichloromethane 1:9, then 17:83, finally 25:75) to give 3-methylquinudidine-3-thiol (495.1 mg, 24%). A portion of this product (285.7 mg, 1.817 mmol) was dissolved in methanol and neutralized with 2M HCl (1.1 mL). The resultant mixture was concentrated to dryness and dried in vacuum overnight. The solid was dissolved in methanol (18 mL) and ethyl ether (9 mL) was added to give crystals. The crystals were collected, washed with ethyl ether and dried in vacuum to give the title compound (265.4 mg, 11%). ¹H NMR (300 MHz, CD₃OD) δ3.44-3.27 (m, 7H), 2.63-2.50 (m, 1H), 2.31-2.27 (m, 1H), 2.11-2.06 (m, 1H), 2.06-1.90 (m, 2H), 1.69 (s, 3H). ¹³C NMR (75 MHz, CD₃OD) δ62.6, 46.9, 46.4, 42.8, 34.3, 32.2, 22.4, 21.2. LRMS (APIMS) m/z 158 (MH⁺).

36c. (3-Methylquinudidin-3-yl)nitrosothio hydrochlroide

The product of Example 36b (40.9 mg, 0.211 mmol) was dissolved in hot N,N-dimethylformamide (1.3 mL) and then cooled to room temperature. tert-Butyl nitrite (30.3 mg, 0.294 mmol) was added. The reaction mixture was stirred for 10 minutes. Excess tert-butyl nitrite was removed by vacuum. Ethyl ether (1.3 mL) was added to give a precipitate. The precipitate was collected, washed with ethyl ether, and dried in vacuum to give the title compound (22.2 mg, 47%). ¹H NMR (300 MHz, D₂O) δ4.05-3.89 (m, 2H), 3.60-3.45 (m, 2H), 3.45-3.36 (m, 1H), 3.36-3.25 (m, 1H), 2.71-2.65 (m, 1H), 2.57-2.44 (m, 1H), 2.16-1.98 (m, 3H), 2.11 (s, 3H). ¹³C NMR (75 MHz, D₂O) δ59.4, 54.6, 46.9, 46.5, 30.0, 28.1, 21.4, 20.1. LRMS (APIMS) m/z 187 (MH⁺).

### EXAMPLE 37

2,2-Bis((nitrooxy)methyl)-3-(nitrooxy)propyl 2-(2-(nitrosothio)adamantan-2-yl)acetate

37. To 3-nitrooxy-2,2-bis(nitrooxymethyl)propan-1-ol, prepared according to Example 11c of WO 00/51978 (33.0 mg, 0.122 mmol), in dichloromethane (1 mL) was added 2-(2-(nitrosothio)adamantan-2-yl)acetic acid prepared according to Example 1d of WO 00/28988 (31.6 mg, 0.124 mmol), 1-(3-(dimethylamino)propyl)-3-ethylcarbodiimide hydrochloride (29.8 mg, 0.155 mmol) and 4-dimethylaminopyridine (15.4 mg, 0.126 mmol). The reaction mixture was stirred at room temperature for 1 hour, diluted with dichloromethane, washed with 0.2 M citric acid and brine. The organic phase was dried over magnesium sulfate, filtered, and concentrated. The product was chromatographed (dichloromethane: hexane 1:1) to give the title compound (11.4 mg, 18%). ¹H NMR (300 MHz, CDCl₃) δ4.40(s, 6H), 4.07 (s, 2H), 3.79 (s, 2H), 2.73 (m, 2H), 2.42-2.37 (m, 2H), 2.11-2.07 (m, 3H), 1.99-1.94 (m, 3H), 1.87-1.77 (m, 4H), ¹³C NMR (75 MHz, CDCl₃) δ169.4, 69.1, 65.9, 61.3, 42.1, 41.9, 38.7, 35.6, 33.7, 33.1, 27.1, 27.0. LRMS (APIMS) m/z 526 (MNH₄⁺).

### EXAMPLE 38

2,2-Dimethyl-N-(2-methyl-2-(nitrosothio propyl)-3-(nitrooxy)propanamide

38a. 2-Methyl-2-(nitrosothio)propylamine hydrochloride

To 1-amino-2-methylpropane-2-thiol hydrochloride (5.39 g, 38 mmol) in N,N-dimethylformamide (16 mL) in a salt-ice bath (−10° C. to −20° C.) was slowly added tert-butyl nitrite (4.8 mL, 4.16 g, 40.4 mmol) and the resultant solution was stirred in the salt-ice bath for 30 minutes. Dichloromethane (30 mL) was added and then hexane (250 mL) to give crystals. Under argon, the crystals were collected by filtration and washed with dichloromethane. The product was dried in vacuum to give the title compound (1.69 g, 15%). ¹H NMR (D₂O) δ3.88 (s, 2H), 1.95 (s, 6H). ¹³C NMR (DMSO-d₆) δ54.7, 48.5, 26.5. LRMS (APIMS) m/z 135 (MH⁺).

38b. 2,2-Dimethyl-N-(2-methyl-2-(nitrosothio)propyl)-3-(nitrooxy)propanamide

To 2,2-dimethyl-3-(nitrooxy)propanoic acid prepared according to Example 3 of U.S. Pat. No. 5,428,061 (41.1 mg, 0.252 mmol) in dichloromethane (1 mL) was added triethylamine (34.1 mg, 0.337 mmol) and isobutyl chloroformate (36.3 mg, 0.266 mmol). The reaction mixture was stirred at room temperature for 17 minutes. Triethylamine (43.6 mg, 0.430 mmol) and the product of Example 38a (56.7 mg, 0.332 mmol) were added. The reaction mixture was then stirred for 3 minutes, diluted with dichloromethane, and washed with 0.2 M citric acid and brine. The organic phase was dried over magnesium sulfate, filtered and concentrated. The product was chromatographed to give the title compound (11.8 mg, 17%). ¹H NMR (300 MHz, CDCl₃) δ6.07(br s, 1H), 4.48 (s, 2H), 4.07 (d, J=6.2 Hz, 2H), 1.88 (s, 6H), 1.24 (s, 6H). ¹³C NMR (75 MHz, CDCl₃) δ174.3, 78.2, 57.2, 49.7, 42.1, 26.7, 22.5. LRMS (APIMS) m/z 280 (MH⁺), 297 (MNH₄⁺).

### EXAMPLE 39

N-(2-Methyl-2-(nitrosothio)propyl)benzamide

39a. N-(2-Methyl-2-sulfanylpropyl)benzamide

To a suspension of 1-amino-2-methylpropane-2-thiol hydrochloride (779.0 mg, 5.499 mmol) in dichloromethane was added potassium hydroxide solution (0.37 g in 1.6 mL). The mixture was shaken vigorously and the organic phase was separated and dried over magnesium sulfate, filtered and concentrated to just dryness to give 1-amino-2-methylpropane-2-thiol (568.1 mg, 98%). To a portion of the 1-amino-2-methylpropane-2-thiol (262.6 mg, 2.496 mmol) was added benzoic acid (297.3 mg, 2.434 mmol), dichloromethane and 1-(3-(dimethylamino)propyl)-3-ethylcarbodiimide hydrochloride (571.0 mg, 2.979 mmol). The reaction mixture was stirred at room temperature overnight, concentrated to dryness, diluted with ethyl acetate. The ethyl acetate solution was washed with 0.2 M citric acid, brine, sodium bicarbonate solution and brine. The organic phase was dried over magnesium sulfate, filtered and concentrated. The resultant product was chromatographed (methanol:dichloromethane 1:99) to give the title compound (380.5 mg, 75%). ¹H NMR (300 MHz, CDCl₃) δ7.83-7.81 (m, 2H), 7.52-7.43 (m, 3H), 6.68 (br s, 1H), 3.54 (d, J=6.1 Hz, 2H), 1.69 (s, 1H), 1.43 (s, 6H). ¹³C NMR (75 MHz, CDCl₃) δ167.5, 134.4, 131.5, 128.5, 126.9, 52.4, 45.7, 30.0. LRMS (APIMS) m/z 210 (MH⁺).

39b. N-(2-Methyl-2-(nitrosothio)propyl)benzamide

To the product of Example 39a (203.7 mg, 0.9732 mmol) in dichloromethane was added tert-butyl nitrile (407 mg, 3.95 mmol). The reaction mixture was stirred at room temperature for 25 minutes, concentrated to dryness, diluted with dichloromethane. The resultant solution was washed with water and brine. The organic phase was dried over magnesium sulfate, filtered and concentrated. The resultant product was chromatogrphed (dichlormethane) to give the title compound (188.2 mg, 81%). ¹H NMR (300 MHz, CDCl₃) δ7.72-7.69 (m, 2H), 7.51-7.46 (m, 1H), 7.42-7.37 (m, 2H), 6.64 (br s, 1H), 4.23 (d, J=6.3 Hz, 2H), 1.94 (s, 6H). ¹³C NMR (75 MHz, CDCl₃) δ167.9, 134.2, 131.6, 128.6, 126.9, 57.5, 49.9, 26.9. LRMS (APIMS) m/z 239 (MH⁺), 256 (MNH₄⁺).

### EXAMPLE 40

2-(2-Methyl-2-(nitrosothio)propyl)isoindoline-1,3-dione

40a. 2-(2-Methyl-2-sulfanylpropyl)isoindoline-1,3-dione

To 1-amino-2-methylpropane-2-thiol (prepared in Example 39a, 305.5 mg, 2.904 mmol) was added phthalic anhydride (344.3 mg, 2.325 mmol) and acetic acid (4 mL). The reaction mixture was stirred at 100° C. overnight, concentrated to dryness and diluted with ethyl acetate. The ethyl acetate solution was washed with 0.2 M citric acid, brine, sodium bicarbonate solution and brine. The organic phase was dried over magnesium sulfate, filtered and concentrated to dryness. The product was chromatographed (neat dichloromethane) to give the title compound (0.35 g, 64%). 1H NMR (300 MHz, CDCl₃) δ7.89-7.86 (m, 2H), 7.76-7.73 (m, 2H), 3.84 (s, 2H), 1.95 (s, 1H), 1.44 (s, 6H). ¹³C NMR (75 MHz, CDCl₃) δ168.4, 134.0, 131.8, 123.3, 50.6, 45.3, 30.9. LRMS (APIMS) m/z 236 (MH⁺), 253 (MNH₄⁺).

40b. 2-(2-Methyl-2-(nitrosothio)propyl)isoindoline-1,3-dione

To the product of Example 40a (200.6 mg, 0.8525 mmol) in dichloromethane was added tert-butyl nitrite (130 mg, 1.26 mmol). The reaction mixture was stirred at room temperature for 30 minutes, concentrated to dryness and diluted with dichloromethane. The dichloromethane solution was washed with water and brine, dried over magnesium sulfate, filtered and concentrated to dryness. The resultant product was chromatogrphed (dichloromethane) to give the title compound (0.2 g, 88%). ¹H NMR (300 MHz, CDCl₃) δ7.89-7.83(m, 2H), 7.77-7.71 (m, 2H), 4.43 (s, 2H), 1.95 (s, 6H). ¹³C NMR (75 MHz, CDCl₃) δ168.4, 134.2, 131.8, 123.5, 56.9, 49.9, 27.5. LRMS (APIMS) m/z 265 (MH⁺), 282 (MNH₄⁺).

### EXAMPLE 41

2-(N-(2-Methyl-2-(nitrosothio)propyl)carbamoyl)benzoic acid

41 a. 2-(N-(2-Methyl-2-sulfanylpropyl)carbamoyl)benzoic acid

To a suspension of 1-amino-2-methylpropane-2-thiol hydrochloride (4.00 g, 28.23 mmol) in dichloromethane (50 mL) at 0° C. was added triethylamine (3.14 g, 31.1 mmol) and phthalic anhydride (4.10 g, 27.7 mmol). The reaction mixture was stirred at room temperature for 1 hour and washed with 2 N hydrochloric acid. The organic phase was concentrated and the product was dried in vacuum to give the title compound (6.36 g, 91%). ¹H NMR (300 MHz, DMSO-d₆) δ10.2 (br s, 11H), 8.44 (t, J=6.2 Hz, 1H), 7.77-7.75 (m, 1H), 7.60-7.47 (m, 2H), 7.44-7.41 (m, 1H), 3.36 (d, J=6.1 Hz, 2H), 2.83 (s, 1H), 1.33 (s, 6H). ¹³C NMR (75 MHz, DMSO-d₆) δ168.9, 167.9, 138.7, 131.2, 130.5, 129.14, 129.10, 127.9, 52.2, 45.0, 29.8. LRMS (APIMS) m/z 254 (MH⁺).

41b. 2-(N-(2-Methyl-2-(nitrosothio)propyl)carbamoyl)benzoic acid

To the product of Example 41a (1.00 g, 3.95 mmol) in dichloromethane (25 mL) was added tert-butyl nitrite (404 mg, 3.91 mmol). The reaction mixture was stirred at room temperature for 30 minutes and concentrated to dryness. The resultant solid was triturated with small amount of ethyl ether and hexane. The solid was collected and dried in vacuum to give the title compound (1.11 g, 100%). ¹H NMR (300 MHz, DMSO-d₆) δ7.92-7.90 (m, 1H), 7.85 (t, J=6.2 Hz, 1H), 7.55-7.43 (m, 3H), 4.16 (d, J=6.4 Hz, 2H), 1.98 (s, 6H). ¹³C NMR (75 MHz, DMSO-d₆) δ169.7, 167.6, 137.9, 130.9, 129.5, 129.4, 128.7, 127.3, 57.1, 49.1, 26.2. LRMS (APIMS) m/z 283 (MH⁺).

### EXAMPLE 42

4-(4-(2-Methyl-2-(nitrosothio)propyl)piperazinyl)benzcarbonitrile

42a. 4-Piperazinylbenzenecarboonitrile

4-Fluorobenzonitrile (15.87 g, 0.131 mol), potassium carbonate (90.55 g, 0.655 mol) and piperazine (33.8 g, 0.393 mol) were slurried together in dry toluene (250 mL). The resulting mixture was heated to reflux for 3 days, cooled to ambient temperature, diluted with ethyl acetate. The reaction mixture was washed with water (3×), brine and then extracted with ethyl acetate (2×). The combined organic layers were dried over sodium sulfate, filtered and the solvent removed in vacuo to give the title product (21.8 g, 89%) as an off-white solid: ¹H NMR (CDCl₃) δ7.48 (m, 2H), 6.85 (m, 2H), 3.27 (m, 4H), 3.00 (m, 4H), 1.67 (s, 1H).

42b. 4-(4-(2-Methyl-2-sulfanylpropyl)piperazinyl)benzenecarbonitrile

To the product of Example 42a (5.51 g, 29.4 mmol) in dry toluene (20 mL) was added 2,2-dimethylthirane (2.72 g, 30.9 mmol) and the resulting mixture was heated at 80° C. for 3 days. The reaction mixture was cooled to ambient temperature and the solvent removed in vacuo to give a thick yellow oil. The residue was chromatographed, eluting with methylene chloride (400 mL), 2:98 ethanol/methylene chloride (250 mL) 1:9 ethanol/methylene chloride and 1:1 ethanol/methylene chloride (250 mL). Concentration of the appropriate fractions gave the title compound (3.86 g, 48%) as an off-white solid. Mp 92-94° C.; ¹H NMR (CDCl₃) δ7.48 (m, 2H), 6.84 (m, 2H), 3.31 (m, 4H), 2.80 (m, 4H), 2.46 (s, 2H), 1.34 (s, 6H); LRMS (APIMS) m/z 276 (MH⁺).

42c. 4-(4-(2-Methyl-2-(nitrosothio)propyl)piperazinyl)benzcarbonitrile

The product of Example 42b (630 mg, 2.29 mmole) was dissolved in methylene chloride (3 mL ), then methanol saturated with HCl (6 mL) was added at ambient temperature to give a clear pale yellow solution. tert-Butyl nitrite (248 mg, 2.40 mL, 0.32 mL) was added at ambient temperature and the resulting mixture was stirred for 1 hour at which point TLC showed the reaction was complete. The solvent was removed in vacuo to give a green foam. The foam was triturated thrice with ether and dried under vacuum to give the title compound as the hydrochloride salt (770 mg, 98%) as a pale green solid. Mp 70° C. (dec). ¹H NMR (DMSO-d₆) δ7.64 (m, 2H), 7.08 (m, 2H), 4.15-3.30 (bm, 8H), 2.14 (bs, 2H), 1.11 (s, 6H); LRMS (APIMS) m/z 305 (M+1)⁺.

A small sample of the hydrochloride salt (240 mg) was neutralized with saturated sodium bicarbonate and then extracted with dicloromethane. The organic layer was dried over sodium sulfate, filtered and the solvent removed in vacuo to give the title compound (170 mg, 79%) as a green semi-solid. ¹H NMR (CDCl₃) δ7.48 (m, 2H), 6.83 (m, 2H), 3.27 (m, 4H), 3.06 (s, 2H), 2.76 (m, 4H), 1.91 (s, 6H); LRMS (APIMS) m/z 305 (M+1)⁺.

### EXAMPLE 43

N-(2-(Dimethylbenzylammonium)ethyl)-2-(2-(nitrosothio)adamantan-2-yl)acetamide chloride

43a. 2-(2-Acetylthioadamantan-2-yl)-N-(2-(dimethylamino)ethyl)acetamide

To the product of Example 34a (1.66 g, 6.13 mmol) in chloroform (40 mL) was added oxalyl chloride (1.05 g, 8.25 mmol) and N,N-dimethylformamide (23 JIL). The reaction mixture was stirred at room temperature for 1 hour, concentrated to dryness in vacuum. The resultant oil was dissolved in chloroform (40 mL) and (2-aminoethyl)dimethylamine (0.6 g, 6.83 mmol) was added. The reaction mixture was stirred at room temperature overnight, washed with potassium hydroxide solution (0.42 g, 7.52 mmol) and brine, dried over sodium sulfate, filtered and concentrated. The product was chromatographed (methanol:dichloromethane 1:20) to give the title compound (1.71 g, 82%). ¹H NMR (300 MHz, CDCl₃) δ6.06 (br s, 1H), 3.29 (q, J=5.7 Hz, 2H), 3.19 (s, 2H), 2.46 (m, 2H), 2.38-2.36 (m, 3H), 2.29-2.27 (m, 4H), 2.23-2.16 (m, 8H), 1.73 (m, 2H), 1.73-1.63 (m, 6H). ¹³C NMR (75 MHz, CDCl₃) δ197.7, 170.8, 62.6, 57.7, 45.0 40.5, 39.0, 36.5, 34.0, 33.7, 32.8, 31.7, 27.1, 27.0. LRMS (APIMS) m/z 339 (MH⁺).

43b. N-(2-(Dimethylbenzylammonium)ethyl)-2-(2-sulfanyladamantan-2-yl)acetamide chloride

To the product of Example 43a (222.7 mg, 0.66 mmol) in dichloromethane (1 mL) was added benzyl chloride (972 mg, 7.68 mmol). The reaction mixture was stirred at room temperature overnight. The solid was collected by filtration, washed with dichloromethane and dried to give a white solid (298.8 mg). This white solid (278.7 mg) in methanol was saturated with ammonia at 0° C. The flask was capped tightly. The reaction mixture was stirred at room temperature for 2 hours and at open air for 10 minutes and concentrated to dryness. The product was chromatographed (methanol:dichloromethane:ammonium hydroxide 15:85:1) to give the title compound. ¹H NMR (300 MHz, CD₃OD) δ7.62-7.49 (m, 5H), 4.63 (s, 2H), 3.52-3.47 (m, 2H), 3.34-3.30 (m, 2H), 3.13 (s, 6H), 2.88 (s, 2H), 2.52-2.49 (m, 2H), 2.24-2.17 (m, 2H), 1.96-1.94 (m, 2H), 1.83-1.28 (m, 8H). ¹³C NMR (75 MHz, CD₃OD) δ174.2, 134.3, 132.0, 130.3, 128.7, 69.7, 63.5, 55.4, 50.72, 50.67, 50.62, 48.0, 40.2, 39.5, 34.9, 34.33, 34.27, 29.1, 28.4. LRMS (APIMS) m/z 387 (M⁺-Cl).

43c. N-(2-(Dimethylbenzylammonium)ethyl)-2-(2-(nitrosothio)adamantan-2-yl)acetamide chloride

To the product of Example 43b (133 mg, 0.32 mmol) in methanol (1 mL) was added dichloromethane (2 mL) and tert-butyl nitrite (120 mL, 106 mg, 1.03 mmol). The solution was stirred at room temperature for 30 minutes in the dark, concentrated to dryness, and chromatographed (methanol:dichloromethane 15:85) to give the title compound (121 mg, 85%), which was further crystallized from chloroform. ¹H NMR (CDCl₃) δ8.1 (br s, 1H), 7.5 (m, 5H), 4.5 (s, 2H), 3.65 (s, 2H), 3.57 (m, 2H), 3.3 (m, 2H), 3.0 (s, 6H), 2.8 (m, 2H), 2.5 (m, 2H), 2.1-1.6 (m, 10H). ¹³C NMR (CDCl₃) δ171.2, 133.0, 130.6, 129.1, 126.9, 68.1, 67.1, 62.9, 49.8, 43.4, 38.7, 35.4, 33.7, 33.0, 27.04, 26.98. LRMS (APIMS) m/z 416 (M⁺-Cl).

### EXAMPLE 44

2-(2-(Nitrosothio)adamantan-2-yl)-N-(2-(trimethylammonium)ethyl)-acetamide chloride

44a. 2-(2-Acetylthioadamantan-2-yl)-N-(2-(trimethylammonium)ethyl)acetamide iodide

To the product of Example 43a (301 mg, 0.89 mmol) in dichloromethane (5 mL) was added iodomethane (1 mL, 2.28 g, 16.1 mmol). The solution was stirred at room temperature for 30 minutes and the precipitate collected by filtration, washed with dichloromethane and dried in vacuum to give the title compound (416 mg, 97%). ¹H NMR (CDCl₃) δ4.82 (s, 2H), 3.6 (m, 2H), 3.5 (m, 2H), 3.2 (s, 9H), 2.5-2.1 (m, 9H), 1.9-1.6 (m, 8H). ¹³C NMR (CDCl₃) δ198.0, 173.8, 65.6, 63.3, 54.23, 54.18, 54.13, 41.5, 40.1, 35.3, 34.73, 34.66, 33.8, 32.0, 28.7, 28.6.

44b. 2-(2—Sulfanyladamantan-2-yl)-N-(2(trimethylammonium)ethyl)acetamide chloride

To the product of Example 44a (1.52 g, 3.16 mmol) in methanol was added a silver nitrate solution (600 mg in water). The solution was stirred for 3 seconds and brine (2.5 mL) was added. The precipitate was removed and washed with methanol. The filtrate in an ice-water bath was saturated with ammonia gas and the flask sealed. The solution was stirred at room temperature for 2 hours, concentrated to dryness, and chromatographed (methanol:dichloromethane 1:4 to methanol:dichloromethane 1:1) to give the title compound (698 mg, 64%). ¹H NMR (CD₃OD) δ3.68 (m, 2H), 3.52 (m, 2H), 3.23 (m, 9H), 2.91 (s, 2H), 2.52 (m, 2H), 2.21 (m, 2H), 1.98 (m, 2H), 1.8-1.6 (m, 8H). ¹³C NMR (CD₃OD) δ174.2, 65.8 (t, J=2.8 Hz), 55.3, 54.1 (t, J=3.8 Hz), 48.0, 40.2, 39.5, 34.9, 34.6, 34.3, 29.1, 28.4. LRMS (APIMS) m/z 311 (M⁺-Cl).

44c. 2-(2-(Nitrosothio)adamantan-2-yl)-N-(2-(trimethylammonium)ethyl)-acetamide chloride

To the product of Example 44b (201 mg, 0.58 mmol) in methanol (20 mL) was added tert-butyl nitrite (220 μL, 194 mg, 1.88 mmol). The reaction mixture stirred at room temperature in the dark for 15 minutes and concentrated to dryness. The solid was dissolved in methanol, concentrated to a viscous oil, treated with chloroform (1 mL) and stored at 4° C. to give crystals which were collected by filtration and dried in vacuum to give the title compound (194 mg, 88 %). ¹H NMR (CD₃OD): δ3.66 (s, 2H), 3.49 (m, 2H), 3.31 (m, 2H), 3.12 (s, 9H), 2.87 (m, 2H), 2.53 (m, 2H), 2.1-1.8 (m, 10H). ¹³C NMR (CD₃OD) δ173.2, 68.1, 65.5 (t, J=2.9 Hz), J=3.8 Hz), 44.7, 40.0, 37.2, 36.5, 34.8, 34.5, 34.1, 28.8. LRMS (APIMS) m/z 340 (M⁺-Cl).

### EXAMPLE 45

2(1-Nitrosomercaptocyclohex-1-yl)-1,3-dioxolane

45a. 2(1-Mercaptocyclohex-1-yl)-1,3-dioxolane

A mixture of the product of Example 33a (2 g, 7.3 mmol), ethylene glycol (1.7 g, 28.1 mmol), p-toluenesulfonic acid (0.16 g) and anhydrous magnesium sulfate (2 g) in benzene (50 mL) was refluxed for 16 hours. The white solid was removed by filtration and the filtrate was washed with water, dried over sodium sulfate and concentrated in vacuo. The residue was chromatographed on silica gel, eluting with 1:5 ethyl acetate:hexane to give 1-mercaptocyclohexane-1-carboxaldehyde diuslfide mono-1,3-dioxlane (1.8 g, 80%) as an oil. To a stirred solution of this (1.8 g) in dry THF (20 mL) was added dropwise a solution of lithium aluminum hydride (8.5 mL of 1M solution in tetrahydrofuran, 8.5 mmol) at 0° C. under nitrogen and the resulting solution was stirred at room temperature for 30 minutes. The excess lithium aluminum hydride was destroyed carefully by the addition of sodium sulfate decahydrate and the granular white precipitate was filtered and washed with ethyl acetate. The filtrate was dried over sodium sulfate and concentrated in vacuo. The residue was chromatographed on silica gel eluting with 1:5 ethyl acetate:hexane to give the title compound (0.42 g, 40%). ¹H NMR (300 MHz, CDCl₃) δ4.73 (s, 1H), 3.88-4.08 (m, 4H), 1.54-1.77 (m, 1H), 1.10-1.27 (m, 1H). ¹³C NMR (75 MHz, CDCl₃) δ109.4, 65.8, 52.6, 33.6, 26.1, 21.5. mass spectrum (API-TIS) m/z 189 (MH⁺).

45b. 2(1-Nitrosomercaptocyclohex-1-yl)-1,3-dioxolane

A solution of the product of Example 45a (0.4 g, 2.12 mmol) in dichloromethane (2 mL) was added dropwise to a solution of tert-butyl nitrite (0.69 g, 6.72 mmol) in dichloromethane (1 mL) at room temperature. The resulting solution was stirred for 30 minutes at room temperature in the dark. The residue after evaporation of the solvent was chromatographed on silica gel, eluting with 1:99 ethyl acetate:hexane to give the title compound (0.24 g, 52%) as a low melting solid. Mp 37-39° C. ¹H NMR (300 MHz, CDCl₃) δ5.30 (s, 1H), 3.83-3.93 (m, 4H), 2.61-2.65 (m, 2H), 2.05-2.15 (m, 2H), 1.55-1.67 (m, 6H). ¹³C NMR (75 MHz, CDCl₃) δ108.6, 65.8, 64.6, 30.7, 25.8, 21.6. mass spectrum (API-TIS) m/z 218 (MH⁺). Anal. Calcd for C₉H₁₅NO₃S: C, 49.75; H, 6.96; N, 6.45; S, 14.75. Found: C, 49.42; H, 6.89; N, 5.87; S, 14.32.

### EXAMPLE 46

2-(1-Nitrosomercaptocyclohex-1-yl)-1,3-dioxane

46a. 2-(1-Mercaptocyclohex-1-yl)-1,3-dioxane

A mixture of the product of Example 33a (5 g, 17.6 mmol), 1,3-propanediol (12.6 mL, 13.3 g, 175 mmol), p-toluenesulfonic acid (0.4 g) and anhydrous magnesium sulfate (10 g) in benzene (75 mL) was heated at 60° C. for 2 days. The white solid was removed by filtration and the filtrate was washed with water, dried over sodium sulfate, filtered and evaporated to give the product (5.42 g) as a mixture of 2-mercaptocyclohexane carboxaldehyde disulfide bis-1,3-dioxane and 2-mercaptocyclohexanecarboxaldehyde disulfide mono-1,3-dioxane which was used directly in the next step without further purification. To a stirred solution of this mixture (5.42 g) in dry THF (30 mL) was added dropwise a solution of lithium aluminum hydride (35 mL of 1M solution in THF, 35 mmol) at 0° C. under nitrogen. The resulting solution was stirred at room temperature for 1 hour and then refluxed for 2 hours and after cooling to room temperature, the excess lithium aluminum hydride was destroyed carefully by the addition of sodium sulfate decahydrate. The granular white precipitate was filtered and washed with ethyl acetate. The filtrate was dried over anhydrous Na₂SO₄, filtered and concentrated in vacuo and the residue was chromatographed on silica gel eluting with 1:19 ethyl acetate:hexane to give the title compound (1.23 g, 23%). ¹H NMR (300 MHz, CDCl₃. δ4.60 (s, 1H), 4.03-4.26 (m, 2H), 3.77-3.84 (m, 2H), 2.02-2.18 (m, 1H), 1.92 (s, 1H), 1.44-1.74 (m, 9H), 1.17-1.37 (m, 2H). ¹³C NMR (75 MHz, CDCl₃) δ106.6, 67.2, 52.1, 34.0, 26.1, 25.9, 21.7. mass spectrum (API-TIS) m/z 203 (MH⁺). Anal. Calcd for C₁₀H₁₈O₂S: C, 59.37; H, 8.97; S, 15.85. Found: C, 59.53; H, 8.96; S, 15.76.

46b. 2-(1-Nitrosomercaptocyclohex-1-yl)-1,3-dioxane

The title compound (0.57 g, 74%) was prepared from the product of Example 46a (0.77 g, 3.8 mmol) in dichloromethane (2 mL) and tert-butyl nitrite (0.78 g, 7.6 mmol) in dichloromethane (2 mL) by following the procedure of Example 45b. Mp 34-36° C. ¹H NMR (300 MHz, CDCl₃) δ5.00 (s, 1H), 4.12-4.18 (m, 2H), 3.76-3.84 (m, 2H), 2.56-2.61 (m, 2H), 2.19-2.28 (m, 2H), 1.98-2.12 (m, 1H), 1.32-1.72 (m, 7H). ¹³C NMR (75 MHz, CDCl₃) δ188.4, 105.3, 67.4, 64.3, 30.7, 25.8, 21.9; mass spectrum (API-TIS) m/z 232 (MH⁺). Anal. Calcd for C₁₀H₁₇NO₃S: C, 51.93; H, 7.41; N, 6.06; S, 13.86. Found: C, 51.68; H, 7.45; N, 5.87; S, 13.78.

### EXAMPLE 47

Dimethyl (2,2-dicyclopropyl-2-(nitrosothio)ethyl)phosphonate

47a. Dimethyl (2,2-dicyclopropyl-2-mercaptoethyl)phosphonate

n-Butyl lithium (2.5 M/hexane, 4 mL, 10.0 mmol) was added to a solution of dimethyl methylphosphonate in THF (20 mL) at −78° C. A solution of dicyclopropylmethanethione (1.03 g, 8.2 mmol) was added to the reaction mixture and the temperature was warm up to −50° C. (about 45 minutes), and then stirred at room temperature for 15 minutes. The reaction mixture was quenched with 1N HCl (10 mL) and extracted with dichloromethane (50 mL×3). The combined organic extracts were dried over sodium sulfate, filtered, concentrated and dried under vacuum. The product was chromatographed on silica get eluting with ethyl acetate/hexane (4:1, Rf=0.25) to give the title compound as a clear oil (1.44 g, 70%). ¹H NMR (CDCl₃) δ3.75 (d, J_{PH}=11.0 Hz, 6 H), 2.29 (d, J_{PH}=18.6 Hz, 2 H), 2.20 (d, J_{PH}=1.5 Hz, 1 H), 1.2-1.1 (m, 2H), 0.6-0.4 (m, 8H). ¹³C NMR (CDCl₃) δ52.0 (d, J_{PC}=6.8 Hz), 48.5 (d, J_{PC}=2.3 Hz), 39.9 (d, J_{PC}=138.8 Hz), 20.5 (d, J_{PC}=9.3 Hz), 2.7, 0.9. Analysis calcd. for C₁₀H₁₉O₃PS: C, 47.99; H, 7.65; Found: C, 48.17; H, 7.42.

47b. Dimethyl (2,2-dicyclopropyl-2-(nitrosothio)ethyl)phosphonate

tert-Butyl nitrite (14 mL, 1.2 mmol) was added to an ice-cold mixture of the product of Example 47a (0.25 g, 0.96 mmol) and 1N HCl (2 mL). The reaction mixture was stirred in an ice-bath for 1 hour and at room temperature for 1.5 hours. Water (30 mL) was added and the mixture was extracted with dichloromethane (30 mL×2). The combined organic extracts were dried over Na₂SO₄, filtered, concentrated and dried under vacuum. The product was chromatographed on silica gel, eluting with ethyl acetate to give the title compound as green oil (0.23 g, 86%). ¹H NMR (CDCl₃) δ3.74 (d, J_{PH}=11.0 Hz, 6 H), 3.11 (d, J_{PH}=19.3, 2 H), 1.8-1.7 (m, 2H), 0.7-0.5 (m, 8H). ¹³C NMR (CDCl₃) δ60.6, 52.1 (d, J_{PC}=6.6 Hz), 36.7 (d, J_{PC}=139.1 Hz), 18.4 (d, J_{PC}=6.5 Hz), 2.8, 1.1.

### EXAMPLE 48

Dimethoxy ((2-(nitrosothio)adamantan-2-yl)methyl)phosphino-1-one

48a. Dimethoxyphosphino((2-sulfanyladamantan-2-yl)methyl)-1-one

Methyl dimethyl phosphonate (9.03 g, 0.073 mol) was dissolved in dry THF (100 mL) and cooled to −78° C. n-BuLi (0.069 mol, 27.7 mL of a 2.5M solution in hexanes) was added over a period of 10 minutes to give a pale yellow solution that was maintained at −78° C. for 75 minutes. Adamantane thione (9.04 g, 0.054 mole), in dry THF (20 mL) was added over a 15 minute period and the resulting mixture was stirred for 1 hour at −78° C. and then warmed to ambient temperature for 30 minutes. The reaction was quenched by the addition of saturated aqueous NH₄Cl (15 mL), extracted with ethyl acetate and the organic extract was dried over sodium sulfate, filtered and the solvent removed in vacuo to give a yellow oil. The oil was chromatographed on silica gel, eluting with ethyl acetate/hexanes (1:9, 3:7, and 7:3) to give the title compound (4.0 g, 25.5%) as a white solid. Mp 44-45° C.; ¹H NMR (CDCl₃) δ3.69 (d, J=12.3 Hz, 6H), 3.15 (s, 1H), 2.53 (d, J=19.6 Hz, 2H), 2.46 (m, 2H), 2.01 (m, 2H), 1.91 (m, 2H), 1.79 (m, 2H), 1.77-1.54 (m, 6H); LRMS (APIMS) m/z 308 (MNH₄⁺).

48b. Dimethoxy ((2-(nitrosothio)adamantan-2-yl)methyl)phosphino-1-one

To the product of Example 48a (135 mg, 0.46 mmol) in dicloromethane (2.5 mL) was added tert-butyl nitrite (58 mg, 0.56 mmol, 66 μL). The reaction mixture was stirred at ambient temp for 15 minutes. The reaction mixture was directly applied to TLC plates and eluted with ethyl acetate/hexanes (2×1:1). Extraction into ethyl acetate, filtration and removal of solvent in vacuo gave the title compound (110 mg, 75%) as a viscous dark green oil: ¹H NMR (CDCl₃) δ3.52 (d, J=11.0 Hz, 6H), 3.27 (d, J=19.8 Hz, 2 H), 2.74 (m, 2H), 2.34 (m, 2H), 2.01 (m, 3H), 1.86 (m, 3H), 1.77 (m, 2H), 1.67 (m, 2H); LRMS (APIMS) m/z 337 (MNH₄⁺).

### EXAMPLE 49

((2-(Nitrosothio)adaman-2-yl)methylphosphonic acid

49a. 2-(Phosphinomethyl)adamantan-2-thiol

To the product of Example 48a (328 mg, 1.13 mmol) in dicloromethane (10 mL) at 0° C. under argon was added boron tribromide (1.70 g, 6.78 mmol). The reaction mixture was stirred at 0° C. for 1 hour and then slowly warmed to ambient temperature overnight. The reaction mixture was then cooled back to 0° C. and MeOH (2 mL) was added cautiously. After the addition was complete, the reaction mixture was warmed to ambient temperature for 1 hour. The solvent was removed in vacuo to give the title compound (146 mg, 49.3%) as an off-white solid. Mp 160° C. (dec.). ¹H NMR (CDCl₃) δ9.01 (vbs, 3H), 2.80 (d, J=20.8 Hz, 2H), 2.50 (m, 2H), 2.09 (m, 2H), 1.98 (m, 2H), 1.87 (m, 2H), 1.75-1.63 (m, 6H); LRMS (APIMS) m/z 280 (MNH₄⁺).

49b. ((2-(Nitrosothio)adaman-2-yl)methylphosphonic acid

To the product of Example 49a (133 mg, 0.5 mmole) in methanol (4 mL) was added tert-butyl nitrite (55 mg, 0.53 mmol, 70 μL) at ambient temperature. The reaction mixture was stirred at ambient temperature for 2 hours. The solvent was removed in vacuo to give the title compound (130 mg, 88%) as a pale green/yellow foam: ¹H NMR (CDCl₃) δ8.96 (vbs, 2H), 3.32 (d, J=20.4 Hz, 2H), 2.70 (m, 2H), 2.40 (m, 2H), 2.10 (m, 3H), 1.84 (m, 3H), 1.78-1.68 (m, 4H); LRMS (APIMS) m/z 309 (MNH₄⁺).

### EXAMPLE 50

Suppression of Proliferation of Human Coronary Artery Smooth Muscle Cells (CASMC)

Vascular Smooth Muscle Cell (SMC) Antiproliferation Assay

The cells used in this assay were-human coronary artery smooth-muscle cells (CASMC) supplied by Clonetics Corp. (San Diego, Calif.). They were maintained in SmGM-2 growth medium (Clonetics Corp.), which consisted of modified MCDB 131 medium supplemented with 5% (v/v) fetal bovine serum (FBS), 0.5 ng/mL human recombinant epidermal growth factor (EGF), 2 ng/mL human recombinant fibroblast growth factor (FGF), 5 μg/mL bovine insulin, 50 μg/mL gentamicin sulfate, and 50 ng/mL amphotericin B under humidified 95% air-5% CO₂ at 37° C. Cells were used for experiments up to about 17 cumulative population doublings (i.e., passage 9); at this age they still stained positive for smooth muscle actin, a protein marker for smooth muscle cells.

For the SMC antiproliferation assay, the cells were seeded at 3×104 viable cells in 2 mL of SmGM-2 medium per well of a Coming 24 tissue culture well plate (Corning, N.Y.). Stock solutions of the test compounds were prepared just prior to addition to the cells by dissolving in ethanol at a concentration of 1000 times the highest concentration to be assayed. This stock solution was diluted, as required, with ethanol to lower concentrations. On the same day the cells were seeded, but after they had attached and spread out (about 3 hr), each test compound in varying concentrations (2 μL of the diluted stock solutions) was added to four replicate wells (n=4) for each concentration. Control cultures received 2 μL of ethanol per well (n=4). On the following morning, the cultures were examined microscopically and their condition recorded. On the third day after test compound addition (˜68 hr), the cultures were examined microscopically again and the viable cells counted with an hemacytometer following trypsinization with 0.25% trypsin-1 mM EDTA. Trypan Blue dye exclusion was used to discriminate between viable and dead cells. The results were usually presented as % of the control viable cell count (mean±SEM) and were used to determine the IC₅₀ for the inhibition of proliferation of vascular smooth muscle cells. The IC₅₀ for some the nitric oxide donors is given in Table 1.

TABLE 1

| | |
|---|---|
| Non-nitrosylated Compound | Nitrosylated Compound |

| Example # | IC 50 μM | Example # | IC 50 μM |
|---|---|---|---|
| 11a | 80 | 11b | 16 |
| 12c | slight inhibition | 12d | 12 |
| 18a | slight inhibition | 18b | 26 |
| 19a | >200 | 19b | 50 |
| 20c | slight inhibition | 20d | 28 |
| 21b | no inhibition | 21c | 12 |
| 25b | 65 | 25c | 33 |
| 26d | no inhibition | 26e | 33 |
| 27a | 78 | 27b | 34 |
| 12a | 25 | 29 | 11 |
| 34c | no inhibition | 34d | 23 |
| 36b | no inhibition | 36c | 22 |
| not prepared | not tested | 37 | 5 |
| not prepared | not tested | 38a | 40-60 |
| not prepared | not tested | 38b | 50 |
| 43b | no inhibition | 43c | 27 |
| 44b | no inhibition | 44c | 33.5 |
| 45a | slight inhibition | 45b | 42 |
| 46a | no inhibition | 46b | 47 |
| 47a | slight inhibition | 47b | 41 |

Table 1 shows that the nitrosylated (i.e. nitrosothiol) compound inhibits the proliferation of vascular smooth muscle cells.while the correspond non-nitrosylated (i.e. sulfhydryl) derivative either had no inhibition, slight inhibition or had a much higher IC₅₀ for the inhibition of the proliferation of vascular smooth muscle cells. These results indicate that the inhibition of the proliferation of vascular smooth muscle cells was attributable to the presence of the NO moiety.

The disclosure of each patent, patent application and publication cited or described in the specification is hereby incorporated by reference herein in its entirety.

Although the invention has been set forth in detail, one skilled in the art will appreciate that numerous changes and modifications may be made without departing from the spirit and scope of the invention.

What is claimed is:

1. A compound of Formula (I) or Formula (II) or a pharmaceutically acceptable salt thereof:
wherein the compound of Formula (I) is: 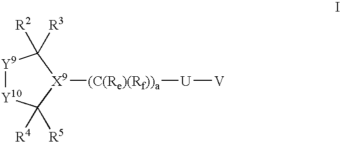
wherein:
X⁹ is CR¹⁰ or nitrogen;
Y⁹ is CR⁶R⁷, NRᵢ, NR²⁵, NRᵢ—CR⁶R⁷, CR⁶R⁷—NRᵢ, CR²R³—CR⁶R⁷ or CR⁶R⁷—CR²R³;
y¹⁰ is CR⁸R⁹ or CR⁸R⁹CR¹⁷R¹⁸;
R², R³, R⁴, R⁵, R⁶, R⁷, R⁸, R⁹, R¹⁷ and R¹⁸ are each independently a hydrogen or an alkyl group; or
R² and R³, R⁴ and R⁵, R⁶ and R⁷ or R⁸ and R⁹ each independently taken together are an oxo; or
R⁴ and R⁷ taken together with the carbon atomss to which they are attached are a cycloalkyl group; or
R⁶ and R⁹ taken together with the carbon atoms to which they are attached are a cycloalkyl group, a bridged cycloalkyl, a heterocyclic ring or an aryl group with the proviso that R⁷ and R³ are not present;
R⁴ and R²⁵ taken together with the carbon and nitrogen atoms to which they are attached are a heterocyclic ring;
R¹⁰ is:
(a) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— U—V;
(b) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— R₃; or
(c) —(C(Rₑ)(R_{f}))ₚ—E_{c}—(C(Rₑ)(R_{f}))ₓ—W_{d}—(C(Rₑ)(R_{f}))_{y}—Wᵢ—E;
a, c, d, g, i and j are each independently an integer from 0 to 3;
p, x, y and z are each independently an integer from 0 to 10;
W at each occurrence is independently —C(O), —C(S), —T, —(C(Rₑ)(R_{f}))ₕ, an alkyl group, an aryl group, a heterocyclic ring, an arylheterocyclic ring, —(CH₂CH₂O)_{q}, a cycloalkyl or a bridged cycloalkyl;
E at each occurrence is independently —T—, an alkyl group, an aryl group, —(C(Rₑ)(R_{f}))ₕ, a heterocyclic ring, an arylheterocyclic ring, —(CH₂CH₂O)_{q}, a carboxylic acid, a carboxylic ester, a nitrile, an amino, a hydroxy or a phosphoryl;
h is an integer form 1 to 10;
q is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, an alkylsulfonyl, an alkylsulfonyloxy, an arylsulfonyl, arylsulphonyloxy, a sulfonic ester, a urea, a nitro, Wₕ, —U—V , or —(C(R_{c})(R_{f}))ₖ—U—V, a phosphoryl; or Rₑ and R_{f} taken together with the carbon atom to which they are attached form a heterocyclic ring, a cycloalkyl group or a bridged cycloalkyl group; or Rₑ and R_{f} taken together are an oxo or a thial;
k is an integer from 1 to 2;
T at each occurrence is independently a covalent bond, a carbonyl, an oxygen, —S(O)ₒ or —N(Rₐ)Rᵢ;
o is an integer from 0 to 2;
U is an oxygen atom, a sulfur atom or —N(Rₐ)(Rᵢ)—;
V is —NO or —NO₂;
Rₐ is a lone pair of electrons, a hydrogen, an alkyl group or an arylalkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an amino alkyl, an amino aryl, —CH₂—C(T-Q)(Rₑ)(R_{f}), a bond to an adjacent atom creating a double bond to that atom, —(N₂O₂—)⁻•M⁺, wherein M⁺ is an organic or inorganic cation;
wherein the compound of Formula (II) is: 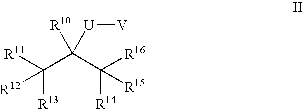
wherein:
R¹¹, R¹², R¹³, R¹⁴, R¹⁵, and R¹⁶ are each independently a hydrogen atom or an alkyl group; or
R¹¹ and R¹² taken together with the carbon atom to which they are attached are a cycloalkyl group or a heterocyclic ring; or
R¹³ and R¹⁴ taken together with the carbon atoms to which they are attached are a cycloalkyl group or a heterocyclic ring; or
R¹⁴ and R¹⁵ taken together with the carbon atom to which they are attached are a cycloalkyl group or a heterocyclic ring; or
R¹¹, R¹² and R¹³ taken together with the carbon atom to which they are attached are a bridged cycloalkyl group; or
R¹⁴, R¹⁵ and R¹⁶ taken together with the carbon atom to which they are attached are a bridged cycloalkyl group; or
R¹¹, R¹², R¹³, R¹⁴, R¹⁵, and R¹⁶ taken together with the carbon atoms to which they are attached are a bridged cycloalkyl group;
R¹⁰, U, and V are as defined herein; and
with the proviso that the compounds of Formulas (I) and (II) do not include 4-aza-4-(2-methyl-2-(nitrosothio)propyl)tricyclo(5.2.1.0<2,6>)dec-8-ene-3,5-dione and the compounds of ACS registry numbers 15459-95-7; 291518-72-4; 159982-34-0; 364590-42-1; 364056-36-0; 364590-41-0; 159982-39-5; 260268-00-6; 364056-69-9; 364057-09-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1, 251369-33-2; and
with the further proviso that the compounds of Formulas (I) and (II) do not contain the following fragments as part of their structure:

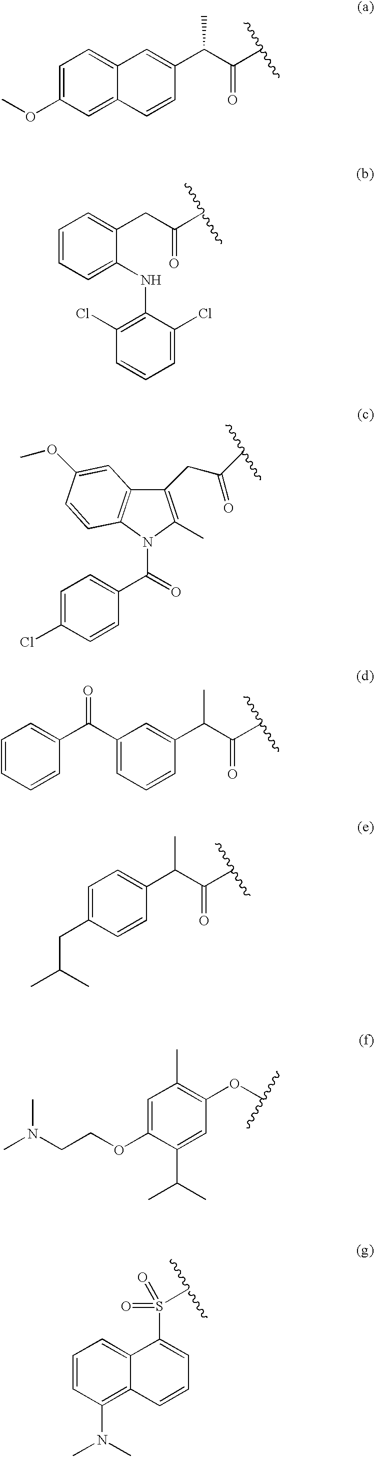

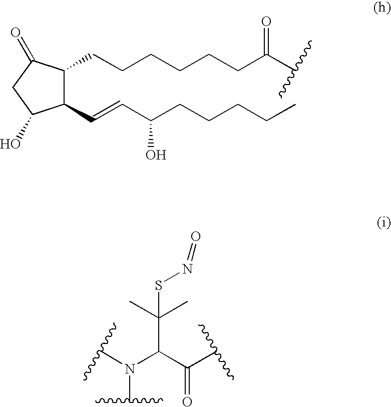

2 The compound of claim 1, wherein the compound of Formula (I) or Formula (II) is:
nitroso(1,1,3,3-tetramethyl-2-prop-2-enylindan-2-yl)thio,
2-(1,1,3,3-tetramethyl-2-(nitrosothio)indan-2-yl)ethan-1-ol,
2-(1,1,3,3-tetramethyl-2-(nitrosothio)indan-2-yl)acetic acid,
2-(1,1,3,3-tetramethyl-2-(nitrosothio)indan-2-yl)ethanenitrile,
2-((N-(2-tethyl-2-(nitrosothio)propyl)carbamoyl)methylthio)acetic acid,
nitrosothio(1,3,3-trimethyl-2-prop-2-enylbicyclo(2.2.1)hept-2-yl,
2-(1,3,3-trimethyl-2-(nitrosothio)bicyclo(2.2.1)hept-2-yl)ethan-1-ol,
2-(1,3,3-trimethyl-2-(nitrosothio)bicyclo(2.2.1)hept-2-yl)ethanenitrile,
(4-methoxyphenyl)-N-(2-(1,3,3-trimethyl-2-(nitrosothio)bicyclo(2.2.1)hept-2-yl)
ethyl)carboxamide,
nitrosothio(1,7,7-trimethyl-2-prop-2-enylbicyclo(2.2.1)hept-2-yl,
2-(2-(nitrosothio)adamantan-2-yl)acetamide,
(1,1-bis(tert-butyl)but-3-enyl)nitrosothio,
4-(tert-butyl)-5,5-dimethyl-4-(nitrosothio)hexan-1-ol,
3-(tert-butyl)-4,4-dimethyl-3-(nitrosothio)pentanenitrile,
(1,1-diadamantanylbut-3-enyl)nitrosothio,
3-(N-(2-methyl-2-(nitrosothio)propyl)carbamoyl)pyrazine-2-carboxylic acid,
(2-methyl-2-(nitrosothio)propyl)(2-methylthiopyrirnidin-4-yl)amine,
4-(N-(2-methyl-2-(nitrosothio)propyl)carbamoyl)butanoic acid,
N-(2-methyl-2(nitrosothio)propyl)((2-methyl-2-(nitrosothio)propyl)amino) carboxamide,
1-(2-methyl-2-(nitrosothio)propyl)imidazolidine-2,4,5-trione,
3-(5-(1-methyl-1-(nitrosothio)ethyl)-3,6-dioxopiperizin-2-yl)propanoic acid,
2-(acetylamino)-N-((2-(nitrosothio)adamantan-2-yl)methyl)acetamide, adamantanylnitrosothio,
(2-methyladamantan-2-yl)nitrosothio,
phenylmethyl 4-(hydroxymethyl)-4-(nitrosothio)piperidinecarboxylate,
4-methyl-4-(N-(2-methyl-2-(nitrosothio)propyl)carbamoyl)pentanoic acid,
N,N-dimethyl-2-(2-(nitrosothio)adamantan-2-yl)acetanide,
tert-butyl 2-(2-(nitrosothio)adamantan-2-yl)acetate,
1,1-dimethyl-2-(4-(2-pyridyl)piperazinyl)ethyl)nitrosothiol,
2-(2-(nitrosothio)adamantan-2-yl)ethyl 4-methoxybenzoate,
(1,1-dimethyl-2-(2-1,2,3,4-tetrahydroisoquinolyl)ethyl)nitrosothio,
4-(N-(((nitrosothiocyclohexyl)methyl)carbamoyl)butanoic acid,
N-(2-hydroxyethyl)-2-(2-(nitrosthio)adamantan-2-yl)acetamide,
N-(2-(2-(nitrosothio)adamantan-2-yl)ethyl)acetamide,
(3-methylquinudidin-3-yl)nitrosothio hydrochloride,
2,2-bis((nitrooxy)methyl)-3-(nitrooxy)propyl 2-(2-(nitrosothio)adamantan-2-yl)acetate,
2,2-dimethyl-N-(2-methyl-2-(nitrosothio)propyl)-3-(nitrooxy)propanamide,
N-(2-methyl-2-(nitrosothio)propyl)benzamide,
2-(2-methyl-2-(nitrosothio)propyl)isoindoline-1,3-dione,
2-(N-(2-methyl-2-(nitrosothio)propyl)carbamoyl)benzoic acid,
4-(⁴-(2-methyl-2-(nitrosothio)propyl)piperazinyl)benzcarbonitrile,
N-(2-(dimethylbenzylammonium)ethyl)-2-(2-(nitrosothio)adamantan-2-yl)acetanide chloride,
2-(2-(nitrosothio)adamantan-2-yl)-N-(2-(trimethylammonium)ethyl)-acetanide chloride,
2(1-nitrosomercaptocyclohex-1-yl)-1,3-dioxolane,
2-(1-nitrosomercaptocyclohex-1-yl)-1,3-dioxane,
dimethyl (2,2-dicyclopropyl-2-(nitrosothio)ethyl)phosphonate,
dimethoxy ((2-(nitrosothio)adamantan-2-yl)methyl)phosphino-1-one,
((2-(ditrosothio)adaman-2-yl)methylphosphonic acid,
3-(N-(2-methyl2-(nitrosothio)propyl)carbamoyl)propanoic acid,
3-(N-(2-ethyl-2-(nitrosothio)butyl)carbamoyl)propanoic acid,
3,3-dimethyl-4-(N-(2-methyl-2-(nitrosothio)propyl)carbamoyl)butanoic acid,
3-(N-(2-methyl-2-(nitrosothio)propyl)-N-benzylcarbamoyl)propanoic acid,
2-(((N-(2-methyl-2-(nitrosothio)propyl)carbamoyl)methyl)cyclopentyl)acetic acid,
(1S,2R)-2-(N-(2-methyl-2-(nitrosothio)propyl)carbamoyl)cyclohexanecarboxylic acid,
(1R,2R)-2-(N-(2-methyl-2-(nitrosothio)propyl)carbamoyl)cyclohexanecarboxylic acid,
3-(N-(2-methyl-2-(nitrosothio)propyl)carbamoyl)-7-oxabicyclo(2.2.1)hept-5-ene-2-carboxylic acid,
3-(N-methyl-N-(2-methyl-2—(nitrosothio)propyl)carbamoyl)propanoic acid,
(tert-butoxy)-N-(2-hydroxy-1-(1-methyl-1-(nitrosothil)ethyl)ethyl)carboamide,
3-(N-(2,2-dimethylpropyl)-N-(2-methyl-2-(nitrosothio)propyl)carbamoyl)propanoic acid or
3-(tert-butyl)-4,4-dimethyl-3-(nitrosthio)pentanenitrile.

3. A composition comprising the compound of claim 1 and a pharmaceutically acceptable carrier.

4. A method for treating a cardiovascular disease or disorder in a patient in need thereof comprising administering a therapeutically effective amount of the composition of claim 3.

5. The method of claim 4, wherein the cardiovascular disease or disorder is restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or non-vascular complication associated with the use of a medical device, a wound associated with the use of a medical device, vascular or non-vascular wall damage, peripheral vascular disease or neoinitimal hyperplasia following percutaneous transluminal coronary angiograph.

6. The method of claim 5, wherein the cardiovascular disease or disorder is restenosis or atherosclerosis.

7. A method for treating a pathological condition resulting from abnormal cell proliferation, a transplant rejection, an autoimmune, inflammatory, proliferative, hyperproliferative or vascular disease, for reducing scar tissue or for inhibiting wound contraction in a patient in need thereof comprising administering a therapeutically effective amount of the composition of claim 3.

8. The method of claim 7, wherein the pathological condition resulting from abnormal cell proliferation is a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia or an acute or chronic granulocytic lymphoma.

9. The method of claim 7, wherein the autoimmune, inflammatory, proliferative, hyperproliferative or vascular disease is rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis, an inflammatory skin disease, an inflammatory lung disease, an inflammatory bowel disease, an inflammatory disease that affects or causees obstruction of a body passageway, an inflammation of the eye, nose or throat, a fungal infection or a food related allergy.

10. The method of claim 4 or 7, wherein the composition is administered intravenously, orally, bucally, parenterally, by an inhalation spray, by topical application or transdermally.

11. The method of claim 4 or 7, wherein the composition is administered via local administration.

12. The method of claim 11, wherein the local administration of the composition is via a suture, a vascular implant, a stent, a heart valve, a drug pump, a drug delivery catheter, an infusion catheter, a drug delivery guidewire or an implantable medical device.

13. A method for direct delivery of nitric oxide to a targeted site in a patient in need thereof comprising administering the composition of claim 3 directly to the targeted site in the patient.

14. The method of claim 13, wherein the composition provides sustained delivery of nitric oxide to the targeted site in the patient.

15. The composition of claim 3, further comprising at least one therapeutic agent or a pharmaceutically acceptable salt thereof.

16. The composition of claim 15, wherein the therapeutic agent is a antithrombogenic agent, a thrombolytic agent, a fibrinolytic agent, a vasospasm inhibitor, a potassium channel activator, a calcium channel blocker, an antihypertensive agent, an antimicrobial agent, an antibiotic, an antiplatelet agent, an antimitotic agent, an antiproliferative agent, a microtubule inhibitor, an antisecretory agent, a remodelling inhibitor, an antisense nucleotide, an anti-cancer chemotherapeutic agent, a steroid, a non-steroidal antiinflammatory agent, a selective COX-2 inhibitor, a 5-lipoxygenase inhibitor, a leukotriene B₄ receptor antagonist, a leukotriene A₄ hydrolase inhibitor, a 5-HT agonist, a HMG-CoA inhibitor, a H₂ receptor antagonist, an antineoplastic agent, a thromboxane inhibitor, a decongestant, a diuretic, a sedating or non-sedating anti-histamine, an inducible nitric oxide synthase inhibitor, an opioid, an analgesic, a Helicobacter pylori inhibitor, a proton pump inhibitor, an isoprostane inhibitor, a vasoactive agent, a β-agonist, an anticholinergic, a mast cell stabilizer, an immunosuppressive agent, a growth factor antagonist or antibody, a dopamine agonist, a radiotherapeutic agent, a heavy metal functioning as a radiopaque agent, a biologic agent, an angiotensin converting enzyme inhibitor, an angiotensin II receptor antagonist, a renin inhibitor, a free radical scavenger, an iron chelator, an antioxidant, a sex hormone, an antipolymerase, an antiviral agent, a photodynamic therapy agent, an antibody targeted therapy agent, a gene therapy agent, or a mixture of two or more thereof.

17. The composition of claim 15, wherein the therapeutic agent has at least one NO group, at least one NO₂ group or at least one NO and NO₂ group, wherein the at least one NO group, at least one NO₂ group or at least one NO and NO₂ group, is linked to the therapeutic agent through an oxygen atom, a nitrogen atom or a sulfur atom.

18. The composition of claim 15, wherein the therapeutic agent is an antiproliferative agent, a steroid, a non-steroidal antiinflammatory agent, an immunosuppressive agent or a mixture of two or more thereof.

19. A method for treating a cardiovascular disease or disorder in a patient in need thereof comprising administering a therapeutically effective amount of the composition of claim 15.

20. The method of claim 19, wherein the cardiovascular disease or disorder is restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or non-vascular complication associated with the use of a medical device, a wound associated with the use of a medical device, vascular or non-vascular wall damage, peripheral vascular disease or neoinitimal hyperplasia following percutaneous transluminal coronary angiograph.

21. The method of claim 20, wherein the cardiovascular disease or disorder is restenosis or atherosclerosis.

22. A method for treating a pathological condition resulting from abnormal cell proliferation, a transplant rejection, an autoimmune, inflammatory, proliferative, hyperproliferative or vascular disease, for reducing scar tissue or for inhibiting wound contraction in a patient in need thereof comprising administering a therapeutically effective amount of the composition of claim 15.

23. The method of claim 22, wherein the pathological condition resulting from abnormal cell proliferation is a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia or an acute or chronic granulocytic lymphoma.

24. The method of claim 22, wherein the autoimmune, inflammatory, proliferative, hyperproliferative or vascular disease is rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis, an inflammatory skin disease, an inflammatory lung disease, an inflammatory bowel disease, an inflammatory disease that affects or causees obstruction of a body passageway, an inflammation of the eye, nose or throat, a fungal infection or a food related allergy.

25. The method of claim 19 or 22, wherein the composition is administered intravenously, orally, bucally, parenterally, by an inhalation spray, by topical application or transdermally.

26. The method of claim 19 or 22, wherein the composition is administered via local administration.

27. The method of claim 26, wherein the local administration of the composition is via a suture, a vascular implant, a stent, a heart valve, a drug pump, a drug delivery catheter, an infusion catheter, a drug delivery guidewire or an implantable medical device.

28. A method for direct delivery of nitric oxide to a targeted site in a patient in need thereof comprising administering the composition of claim 15 directly to the targeted site in the patient.

29. The method of claim 28, wherein the composition provides sustained delivery of nitric oxide to the targeted site in the patient.

30. A composition comprising at least one compound of Formula (I) or Formula (II) or a pharmaceutically acceptable salt thereof, bound to a matrix;
wherein the matrix is a natural polymer, a synthetic polymer, a natural fiber, a synthetic fiber, or a mixture of two or more thereof; and
wherein the compound of Formula (I) is: 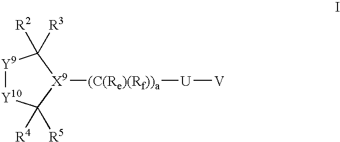
wherein:
X⁹ is CR¹⁰ or nitrogen;
Y⁹ is CR⁶R⁷, NRᵢ, NR²⁵, NRᵢ—CR⁶R⁷, CR⁶R⁷—NRᵢ, CR²R³—CR⁶R⁷ or CR⁶R⁷—CR²R³;
Y¹⁰ is CR⁸R⁹ or CR⁸R⁹CR¹⁷R¹⁸;
R², R³, R⁴, R⁵, R⁶, R⁷, R⁸, R⁹, R¹⁷ and R¹⁸ are each independently a hydrogen or an alkyl group; or
R² and R³, R⁴ and R⁵, R⁶ and R⁷ or R⁸ and R⁹ each independently taken together are an oxo; or
R⁴ and R⁷ taken together with the carbon atoms to which they are attached are a cycloalkyl group; or
R⁶ and R⁹ taken together with the carbon atoms to which they are attached are a cycloalkyl group, a bridged cycloalkyl, a heterocyclic ring or an aryl group with the proviso that R⁷ and R⁸ are not present;
R⁴ and R²⁵ taken together with the carbon and nitrogen atoms to which they are attached are a heterocyclic ring;
R¹⁰ is:
(a) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— U—V;
(b) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— R₃; or
(c) —(C(Rₑ)(R_{f}))ₚ—E_{c}—(C(Rₑ)(R_{f}))ₓ—W_{d}—(C(Rₑ)(R_{f}))_{y}—Wᵢ—E;
a, c, d, g, i and j are each independently an integer from 0 to 3;
p, x, y and z are each independently an integer from 0 to 10;
W at each occurrence is independently —C(O), —C(S), —T, —(C(Rₑ)(R_{f}))ₕ, an alkyl group, an aryl group, a heterocyclic ring, an arylheterocyclic ring, —(CH₂CH₂O)_{q}, a cycloalkyl or a bridged cycloalkyl;
E at each occurrence is independently —T—, an alkyl group, an aryl group, —(C(Rₑ)(R_{f}))ₕ, a heterocyclic ring, an arylheterocyclic ring, —(CH₂CH₂O)_{q}, a carboxylic acid, a carboxylic ester, a nitrile, an amino, a hydroxy or a phosphoryl;
h is an integer form 1 to 10;
q is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylaamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, an alkylsulfonyl, an alkylsulfonyloxy, an arylsulfonyl, arylsulphonyloxy, a sulfonic ester, a urea, a nitro, Wₕ, —U—V , or —(C(Rₑ)(R_{f}))ₖ—U—V, a phosphoryl; or Rₑ and R_{f} taken together with the carbon atom to which they are attached form a heterocyclic ring, a cycloalkyl group or a bridged cycloalkyl group; or Rₑ and R_{f} taken together are an oxo or a thial;
k is an integer from 1 to 2;
T at each occurrence is independently a covalent bond, a carbonyl, an oxygen, —S(O)ₒ or —N(Rₐ)Rᵢ;
o is an integer from 0 to 2;
U is an oxygen atom, a sulfur atom or —N(Rₐ)(Rᵢ)—;
V is —NO or —NO₂;
Rₐ is a lone pair of electrons, a hydrogen, an alkyl group or an arylalkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an amino alkyl, an amino aryl, —CH₂—C(T-Q)(Rₑ)(R_{f}), a bond to an adjacent atom creating a double bond to that atom, —(N₂O₂—)⁻•M⁺, wherein M⁺ is an organic or inorganic cation;
wherein the compound of Formula (II) is: 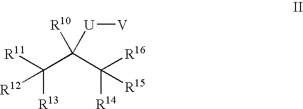
wherein:
R¹¹, R¹², R¹³, R¹⁴, R¹⁵, and R¹⁶ are each independently a hydrogen atom or an alkyl group; or
R¹¹ and R¹² taken together with the carbon atom to which they are attached are a cycloalkyl group or a heterocyclic ring; or
R¹³ and R¹⁴ taken together with the carbon atoms to which they are attached are a cycloalkyl group or a heterocyclic ring; or
R¹⁴ and R¹⁵ taken together with the carbon atom to which they are attached are a cycloalkyl group or a heterocyclic ring; or
R¹¹, R¹² and R¹³ taken together with the carbon atom to which they are attached are a bridged cycloalkyl group; or
R¹⁴, R¹⁵ and R¹⁶ taken together with the carbon atom to which they are attached are a bridged cycloalkyl group; or
R¹¹, R¹², R¹³, R¹⁴, R¹⁵, and R¹⁶ taken together with the carbon atoms to which they are attached are a bridged cycloalkyl group;
R¹⁰, U, and V are as defined herein; and
with the proviso that the compounds of Formulas (I) and (II) do not contain the following fragments as part of their structure:

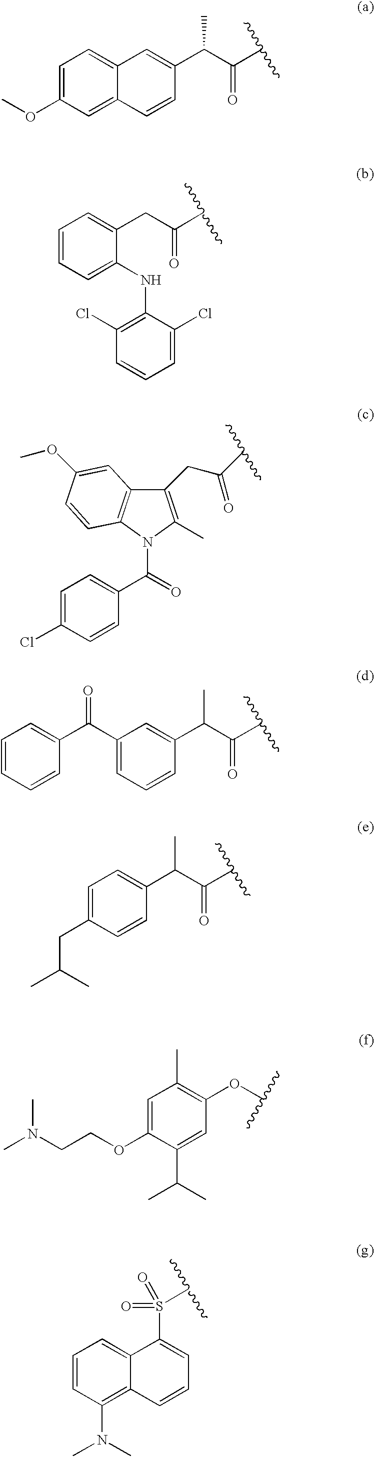

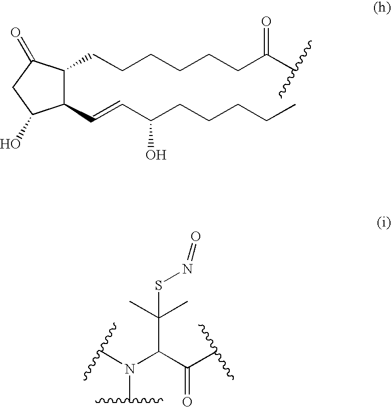

31. The composition of claim 30, wherein the polymer is a polyolefin, a polyethyleneimine derivative, a polyether, a polyester, a polyanhydride, a polyhydroxybutyrate, a polyamide, a polyurethane, a polyurethane copolymer, a polyacrylate, a fluoro substituted polymer, a biopolymer, a starburst dendrimer, or a mixture of two or more thereof.

32. The composition of claim 30, further comprising at least one therapeutic agent or a pharmaceutically acceptable salt thereof.

33. The composition of claim 32, wherein the therapeutic agent has at least one NO group, at least one NO₂ group or at least one NO and NO₂ group, wherein the at least one NO group, at least one NO₂ group or at least one NO and NO₂ group, is linked to the therapeutic agent through an oxygen atom, a nitrogen atom or a sulfur atom.

34. A method for direct delivery of nitric oxide to a targeted site in a patient in need thereof comprising administering the composition of claim 30 or 32 directly to the targeted site in the patient.

35. The method of claim 34, wherein the composition provides sustained delivery of nitric oxide to the targeted site in the patient.

36. A medical device comprising the composition of claim 30 or 32.

37. The medical device of claim 36, wherein the composition coats all or a portion of the surface of the medical device.

38. The medical device of claim 36, wherein the composition forms all or part of the medical device.

39. The medical device of claim 36, wherein the medical device is an intravascular or extravascular medical device, a balloon, a catheter tip, a prosthetic heart valve, a suture, a surgical staple, a synthetic vessel graft, a stent a vascular or non-vascular graft, a shunt, an aneurysm filler, an intraluminal paving system, a guide wire, an embolic agent, a filter, a drug pump, an arteriovenous shunt, an artificial heart valve, an artificial implant, a foreign body introduced surgically into the blood vessels or at a vascular or non-vascular site, a lead, a pacemaker, an implantable pulse generator, an implantable cardiac defibrillator, a cardioverter defibrillator, a defibrillator, a spinal stimulator, a brain stimulator, a sacral nerve stimulator, a chemical sensor, a breast implant, an interventional cardiology device, a catheter, plastic tubing, a dialysis bag or membrane, a bandage or an external device applied directed to the skin.

40. A method for inhibiting platelet aggregation and platelet adhesion caused by the exposure of blood to a medical device comprising incorporating at least one composition of claim 30 or 32 or a pharmaceutically acceptable salt thereof, into or on the medical device.

41. The method of claim 40, wherein the medical device is an intravascular or extravascular medical device, a balloon, a catheter tip, a prosthetic heart valve, a suture, a surgical staple, a synthetic vessel graft, a stent a vascular or non-vascular graft, a shunt, an aneurysm filler, an intraluminal paving system, a guide wire, an embolic agent, a filter, a drug pump, an arteriovenous shunt, an artificial heart valve, an artificial implant, a foreign body introduced surgically into the blood vessels or at a vascular or non-vascular site, a lead, a pacemaker, an implantable pulse generator, an implantable cardiac defibrillator, a cardioverter defibrillator, a defibrillator, a spinal stimulator, a brain stimulator, a sacral nerve stimulator, a chemical sensor, a breast implant, an interventional cardiology device, a catheter, plastic tubing, a dialysis bag or membrane, a bandage or an external device applied directed to the skin.

42. The method of claim 40, wherein the blood is a blood product or a blood component.

43. A method for treating an injured tissue in a patient in need thereof comprising administering at least one composition of claim 30 or 32 or a pharmaceutically acceptable salt thereof, to the site of the injured tissue in the patient.

44. The method of claim 43, wherein the injured tissue is a blood vessel.

45. The method of claim 43, wherein the composition is administered to the site of the injured tissue via at least one of an intravascular or extravascular medical device, a balloon, a catheter tip, a prosthetic heart valve, a suture, a surgical staple, a synthetic vessel graft, a stent a vascular or non-vascular graft, a shunt, an aneurysm filler, an intraluminal paving system, a guide wire, an embolic agent, a filter, a drug pump, an arteriovenous shunt, an artificial heart valve, an artificial implant, a foreign body introduced surgically into the blood vessels or at a vascular or non-vascular site, a lead, a pacemaker, an implantable pulse generator, an implantable cardiac defibrillator, a cardioverter defibrillator, a defibrillator, a spinal stimulator, a brain stimulator, a sacral nerve stimulator, a chemical sensor, a breast implant, an interventional cardiology device, a catheter, plastic tubing, a dialysis bag or membrane, a bandage or an external device applied directed to the skin.

46. A kit comprising at least one compound of claim 1.

47. The kit of claim 46, further comprising at least one therapeutic agent as a separate component in the kit or in the form of a composition in the kit.

48. The kit of claim 47, wherein the therapeutic agent has at least one NO group, at least one NO₂ group or at least one NO and NO₂ group, wherein the at least one NO group, at least one NO₂ group or at least one NO and NO₂ group, is linked to the therapeutic agent through an oxygen atom, a nitrogen atom or a sulfur atom.

49. A method for treating for treating inflammation, pain, and fever; for decreasing for treating gastrointestinal, renal, respiratory and other toxicities resulting from the use of a drug, for a treating gastrointestinal disorder, for treating an inflammatory disease state or disorder; for treating an ophthalmic disease or disorder; for treating and/or improving a gastrointestinal property of a COX-2 inhibitor; for treating a disorder resulting from elevated levels of cyclooxygenase-2; for improving a cardiovascular properties of a COX-2 inhibitor; for decreasing the recurrence of an ulcer, for improving a gastroprotective property, anti-Helicobacter pylori property or an antacid property of a proton pump inhibitor, for treating a Helicobacter pylori and viral infection, for improving a gastroprotective property of a H₂ receptor antagonist, for treating a microbial infection, a multiple sclerosis, a viral infection, for treating a benign prostatic hyperplasia, hypertension, a congestive heart failure, a variant (Printzmetal) angina, a glaucoma, a neurodegenerative disorder, a vasospastic disease, a cognitive disorder, an urge incontinence or an overactive bladder; for reversing the state of an anesthesia; for treating a disease induced by the increased metabolism of cyclic guanosine 3′,5′-monophosphate (cGMP) and for treating a respiratory disorder in a patient in need thereof comprising administering a therapeutically effective amount of at least compound of Formula (I) or Formula (II) or a pharmaceutically acceptable salt thereof:
wherein the compound of Formula (I) is: 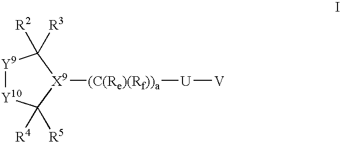
wherein:
X⁹ is CR¹⁰ or nitrogen;
Y⁹ is CR⁶R⁷, NRᵢ, NR²⁵, NRᵢ—CR⁶R⁷, CR⁶R⁷—NRᵢ, CR²R³—CR⁶R⁷ or CR⁶R⁷—CR²R³;
Y¹⁰ is CR⁸R⁹ or CR⁸R⁹CR¹⁷R¹⁸;
R², R³, R⁴, R⁵, R⁶, R⁷, R⁸, R⁹, R¹⁷ and R¹⁸ are each independently a hydrogen or an alkyl group; or
R² and R³, R⁴ and R⁵, R⁶ and R⁷ or R⁸ and R⁹ each independently taken together are an oxo; or
R⁴ and R⁷ taken together with the carbon atoms to which they are attached are a cycloalkyl group; or
R⁶ and R⁹ taken together with the carbon atoms to which they are attached are a cycloalkyl group, a bridged cycloalkyl, a heterocyclic ring or an aryl group with the proviso that R⁷ and R⁸ are not present;
R⁴ and R²⁵ taken together with the carbon and nitrogen atoms to which they are attached are a heterocyclic ring;
R¹⁰ is:
(a) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— U—V;
(b) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}—Rₑ; or
(c) —(C(Rₑ)(R_{f}))ₚ—E_{c}—(C(Rₑ)(R_{f}))ₓ—W_{d}—(C(Rₑ)(R_{f}))_{y}—Wᵢ—E;
a, c, d, g, i and j are each independently an integer from 0 to 3;
p, x, y and z are each independently an integer from 0 to 10;
W at each occurrence is independently —C(O), —C(S), —T, —(C(Rₑ)(R_{f}))ₕ, an alkyl group, an aryl group, a heterocyclic ring, an arylheterocyclic ring, —(CH₂CH₂O)_{q}, a cycloalkyl or a bridged cycloalkyl;
E at each occurrence is independently —T—, an alkyl group, an aryl group, —(C(Rₑ)(R_{f}))ₕ, a heterocyclic ring, an arylheterocyclic ring, —(CH₂CH₂O)_{q}, a carboxylic acid, a carboxylic ester, a nitrile, an amino, a hydroxy or a phosphoryl;
h is an integer form 1 to 10;
q is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, an alkylsulfonyl, an alkylsulfonyloxy, an arylsulfonyl, arylsulphonyloxy, a sulfonic ester, a urea, a nitro, Wₕ, —U—V , or —(C(Rₑ)(R_{f}))ₖ—U—V, a phosphoryl; or Rₑ and R_{f} taken together with the carbon atom to which they are attached form a heterocyclic ring, a cycloalkyl group or a bridged cycloalkyl group; or Rₑ and R_{f} taken together are an oxo or a thial;
k is an integer from 1 to 2;
T at each occurrence is independently a covalent bond, a carbonyl, an oxygen, —S(O)ₒ or —N(Rₐ)Rᵢ;
o is an integer from 0 to 2;
U is an oxygen atom, a sulfur atom or —N(Rₐ)(Rᵢ)—;
V is —NO or —NO₂;
Rₐ is a lone pair of electrons, a hydrogen, an alkyl group or an arylalkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido; a carboxamido, a carboxylic ester, an amino alkyl, an amino aryl, —CH₂—C(T-Q)(Rₑ)(R_{f}), a bond to an adjacent atom creating a double bond to that atom, —(N₂O₂—)⁻•M⁺, wherein M⁺ is an organic or inorganic cation;
wherein the compound of Formula (II) is: 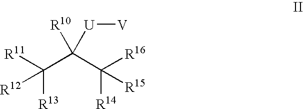
wherein:
R¹¹, R¹², R¹³, R¹⁴, R¹⁵, and R¹⁶ are each independently a hydrogen atom or an alkyl group; or
R¹¹ and R¹² taken together with the carbon atom to which they are attached are a cycloalkyl group or a heterocyclic ring; or
R¹³ and R¹⁴ taken together with the carbon atoms to which they are attached are a cycloalkyl group or a heterocyclic ring; or
R¹⁴ and R¹⁵ taken together with the carbon atom to which they are attached are a cycloalkyl group or a heterocyclic ring; or
R¹¹, R¹² and R¹³ taken together with the carbon atom to which they are attached are a bridged cycloalkyl group; or
R¹⁴, R¹⁵ and R¹⁶ taken together with the carbon atom to which they are attached are a bridged cycloalkyl group; or
R¹¹, R¹², R¹³, R¹⁴, R¹⁵, and R¹⁶ taken together with the carbon atoms to which they are attached are a bridged cycloalkyl group;
R¹⁰, U, and V are as defined herein; and
with the proviso that the compounds of Formulas (I) and (II) do not contain the following fragments as part of their structure:

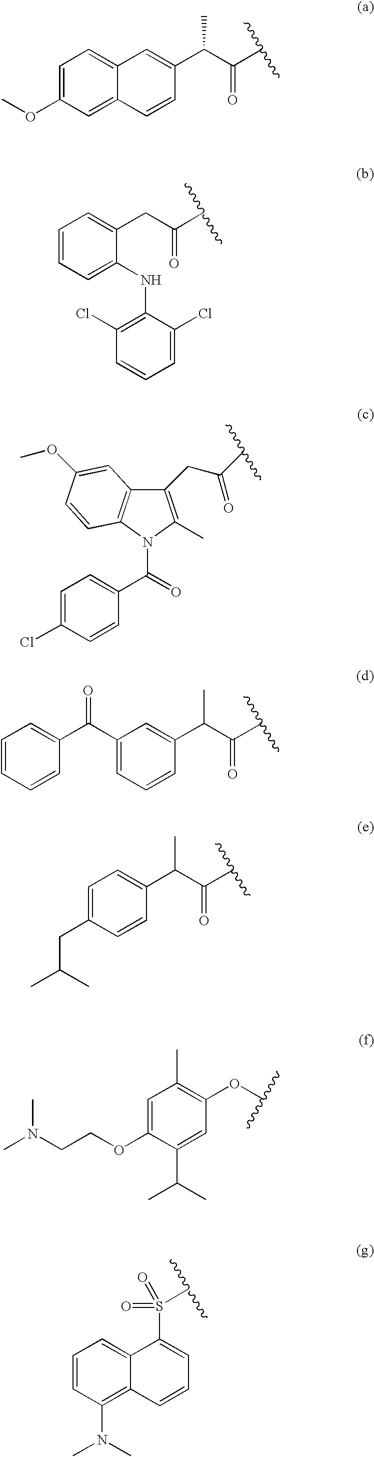

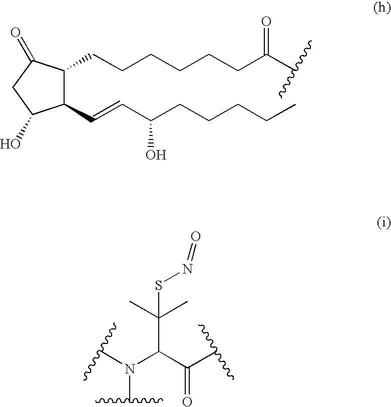

50. A method for treating a sexual dysfunction in a male or female, for enhancing a sexual responses in a male or female patient in need thereof comprising administering a therapeutically effective amount of effective amount of at least one compound of Formula (I) and Formula (II) or a pharmaceutically acceptable salt thereof:
wherein the compound of Formula (I) is: 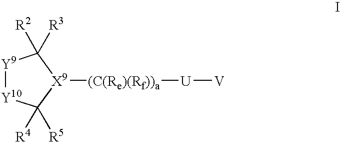
wherein:
X⁹ is CR¹⁰ or nitrogen;
Y⁹ is CR⁶R⁷, NRᵢ, NR²⁵, NRᵢ—CR⁶R⁷, CR⁶R⁷—NRᵢ, CR²R³—CR⁶R⁷ or CR⁶R⁷—CR²R³;
Y¹⁰ is CR⁸R⁹ or CR⁸R⁹CR¹⁷R¹⁸;
R², R³, R⁴, R⁵, R⁶, R⁷, R⁸, R⁹, R¹⁷ and R¹⁸ are each independently a hydrogen or an alkyl group; or
R² and R³, R⁴ and R⁵, R⁶ and R⁷ or R⁸ and R⁹ each independently taken together are an oxo; or
R⁴ and R⁷ taken together with the carbon atoms to which they are attached are a cycloalkyl group; or
R⁶ and R⁹ taken together with the carbon atoms to which they are attached are a cycloalkyl group, a bridged cycloalkyl, a heterocyclic ring or an aryl group with the proviso that R⁷ and R⁸ are not present;
R⁴ and R²⁵ taken together with the carbon and nitrogen atoms to which they are attached are a heterocyclic ring;
R¹⁰ is:
(a) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— U—V;
(b) —(C(Rₑ)(R_{f}))ₚ—E_{c}—(C(Rₑ)(R_{f}))ₓ—W_{d}—(C(Rₑ)(R_{f}))_{y}—Wᵢ—Eⱼ—W_{g}—(C(Rₑ)(R_{f}))_{z}— R₃; or
(c) —(C(Rₑ)(R_{f}))ₚ—E_{c}—(C(Rₑ)(R_{f}))ₓ—W_{d}—(C(Rₑ)(R_{f}))_{y}—Wᵢ—E;
a, c, d, g, i and j are each independently an integer from 0 to 3;
p, x, y and z are each independently an integer from 0 to 10;
W at each occurrence is independently —C(O), —C(S), —T, —(C(Rₑ)(R_{f}))ₕ, an alkyl group, an aryl group, a heterocyclic ring, an arylheterocyclic ring, —(CH₂CH₂O)_{q}, a cycloalkyl or a bridged cycloalkyl;
E at each occurrence is independently —T—, an alkyl group, an aryl group, —(C(Rₑ)(R_{f}))ₕ, a heterocyclic ring, an arylheterocyclic ring, —(CH₂CH₂O)_{q}, a carboxylic acid, a carboxylic ester, a nitrile, an amino, a hydroxy or a phosphoryl;
h is an integer form 1 to 10;
q is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a haloalkoxy, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a haloalkoxy, a sulfonamido, an alkylsulfonamido, an arylsulfonamido, an alkylsulfonyl, an alkylsulfonyloxy, an arylsulfonyl, arylsulphonyloxy, a sulfonic ester, a urea, a nitro, Wₕ, —U—V , or —(C(Rₑ)(R_{f}))ₖ—U—V, a phosphoryl; or Rₑ and R_{f} taken together with the carbon atom to which they are attached form a heterocyclic ring, a cycloalkyl group or a bridged cycloalkyl group; or Rₑ and R_{f} taken together are an oxo or a thial;
k is an integer from 1 to 2;
T at each occurrence is independently a covalent bond, a carbonyl, an oxygen, —S(O)ₒ or —N(Rₐ)Rᵢ;
o is an integer from 0 to 2;
U is an oxygen atom, a sulfur atom or —N(Rₐ)(Rᵢ)—;
V is —NO or —NO₂;
Rₐ is a lone pair of electrons, a hydrogen, an alkyl group or an arylalkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an amino alkyl, an amino aryl, —CH₂—C(T-Q)(Rₑ)(R_{f}), a bond to an adjacent atom creating a double bond to that atom, —(N₂O₂—)⁻•M⁺, wherein M⁺ is an organic or inorganic cation;
wherein the compound of Formula (II) is: 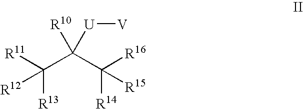
wherein:
R¹¹, R¹², R¹³, R¹⁴, R¹⁵, and R¹⁶ are each independently a hydrogen atom or an alkyl group; or
R¹¹ and R¹² taken together with the carbon atom to which they are attached are a cycloalkyl group or a heterocyclic ring; or
R¹³ and R¹⁴ taken together with the carbon atoms to which they are attached are a cycloalkyl group or a heterocyclic ring; or
R¹⁴ and R¹⁵ taken together with the carbon atom to which they are attached are a cycloalkyl group or a heterocyclic ring; or
R¹¹, R¹² and R¹³ taken together with the carbon atom to which they are attached are a bridged cycloalkyl group; or
R¹⁴, R¹⁵ and R¹⁶ taken together with the carbon atom to which they are attached are a bridged cycloalkyl group; or
R¹¹, R¹², R¹³, R¹⁴, R¹⁵, and R¹⁶ taken together with the carbon atoms to which they are attached are a bridged cycloalkyl group;
R¹⁰, U, and V are as defined herein; and
with the proviso that the compounds of Formulas (I) and (II) do not include 4-aza-4-(2-methyl-2-(nitrosothio)propyl)tricyclo(5.2.1.0<2,6>)dec-8-ene-3,5-dione; and
with the further proviso that the compounds of Formulas (I) and (II) do not contain the following fragments as part of their structure:

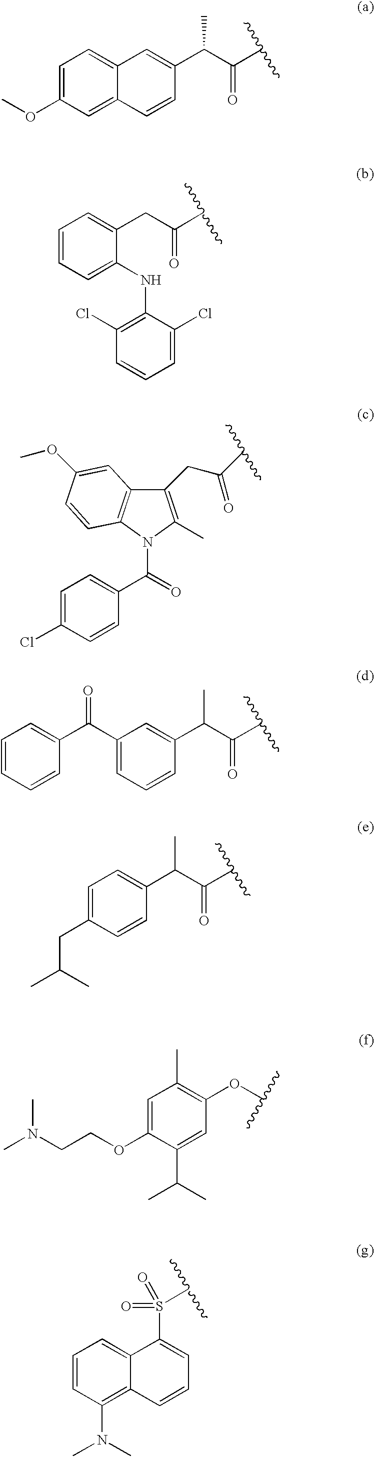

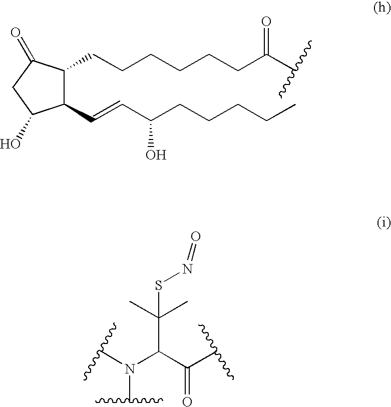

51. The method of claim 49 or 50, further comprising at least one therapeutic agent or a pharmaceutically acceptable salt thereof.

52. The method of claim 51, wherein the therapeutic agent has at least one NO group, at least one NO₂ group or at least one NO and NO₂ group, wherein the at least one NO group, at least one NO₂ group or at least one NO and NO₂ group, is linked to the therapeutic agent through an oxygen atom, a nitrogen atom or a sulfur atom.

53. A method for treating a cardiovascular disease or disorder in a patient in need thereof comprising administering a therapeutically effective amount of a composition comprising at least one of 4-aza-4-(2-methyl-2-(nitrosothio)propyl)tricyclo(5.2. 1.0<2,6>)dec-8-ene-3,5-dione, the compounds of ACS registry numbers 15459-95-7; 291518-72-4; 159982-34-0; 364590-42-1; 364056-36-0; 364590-41-0; 159982-39-5; 260268-00-6; 364056-69-9; 364057-09-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1 and 251369-33-2.

54. The method of claim 53, wherein the cardiovascular disease or disorder is restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or non-vascular complication associated with the use of a medical device, a wound associated with the use of a medical device, vascular or non-vascular wall damage, peripheral vascular disease or neoinitimal hyperplasia following percutaneous transluminal coronary angiograph.

55. The method of claim 55, wherein the cardiovascular disease or disorder is restenosis or atherosclerosis.

56. A method for treating a pathological condition resulting from abnormal cell proliferation, a transplant rejection, an autoimmune, inflammatory, proliferative, hyperproliferative or vascular disease, for reducing scar tissue or for inhibiting wound contraction in a patient in need thereof comprising administering a therapeutically effective amount of the composition of claim 53.

57. The method of claim 56, wherein the pathological condition resulting from abnormal cell proliferation is a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia or an acute or chronic granulocytic lymphoma.

58. The method of claim 56, wherein the autoimmune, inflammatory, proliferative, hyperproliferative or vascular disease is rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis, an inflammatory skin disease, an inflammatory lung disease, an inflammatory bowel disease, an inflammatory disease that affects or causees obstruction of a body passageway, an inflammation of the eye, nose or throat, a fungal infection or a food related allergy.

59. The method of claim 53 or 56, wherein the composition is administered intravenously, orally, bucally, parenterally, by an inhalation spray, by topical application or transdermally.

60. The method of claim 53 or 56, wherein the composition is administered via local administration.

61. The method of claim 60, wherein the local administration of the composition is via a suture, a vascular implant, a stent, a heart valve, a drug pump, a drug delivery catheter, an infusion catheter, a drug delivery guidewire or an implantable medical device.

62. A method for direct delivery of nitric oxide to a targeted site in a patient in need thereof comprising administering the composition of claim 53 directly to the targeted site in the patient.

63. The method of claim 62, wherein the composition provides sustained delivery of nitric oxide to the targeted site in the patient.

64. A composition comprising at least one of 4-aza-4-(2-methyl-2-(nitrosothio)propyl)tricycle (5.2.1.0<2,6>)dec-8-ene-3,5-dione, the compounds of ACS registry numbers 15459-95-7; 291518-72-4; 159982-34-0; 364590-42-1; 364056-36-0; 364590-41-0; 159982-39-5; 260268-00-6; 364056-69-9; 364057-09-0; 72604-09-2; 375371-24-7; 346684-08-0; 346684-04-6; 159982-36-2; 159982-35-1; 159982-37-3; 159982-38-4; 364056-68-8; 72604-10-5; 364590-32-9; 173776-77-7; 364590-39-6; 346683-91-8; 364056-30-4; 364590-35-2; 343271-37-4; 306776-33-0; 306776-44-3; 364056-57-5; 306776-45-4; 306776-46-5; 306776-47-6; 364056-59-7; 306776-52-3; 364056-76-8; 260268-12-0; 260268-15-3; 15459-97-9; 287402-83-9; 287402-85-1; 364057-28-3; 364057-22-7; 204438-82-4; 173776-76-6; 260268-08-4; 260268-05-1; 270248-15-2; 270574-61-3; 287402-87-3; 287402-88-4; 307492-58-6; 364590-45-4; 306776-51-2; 290291-79-1; 364056-34-8; 270248-14-0; 270248-12-9; 364590-98-7; 346683-85-0; 291518-68-8; 364057-32-9; 207607-75-8; 428520-29-0; 251369-34-3; 194597-06-3; 346683-80-5; 346683-72-5; 346683-71-4; 428520-28-9; 260268-21-1 and 251369-33-2 and at least one therapeutic agent or a pharmaceutically acceptable salt thereof.

65. The composition of claim 64, wherein the therapeutic agent is a antithrombogenic agent, a thrombolytic agent, a fibrinolytic agent, a vasospasm inhibitor, a potassium channel activator, a calcium channel blocker, an antihypertensive agent, an antimicrobial agent, an antibiotic, an antiplatelet agent, an antimitotic agent, an antiproliferative agent, a microtubule inhibitor, an antisecretory agent, a remodelling inhibitor, an antisense nucleotide, an anti-cancer chemotherapeutic agent, a steroid, a non-steroidal antiinflammatory agent, a selective COX-2 inhibitor, a 5-lipoxygenase inhibitor, a leukotriene B₄ receptor antagonist, a leukotriene A₄ hydrolase inhibitor, a 5-HT agonist, a HMG-CoA inhibitor, a H₂ receptor antagonist, an antineoplastic agent, a thromboxane inhibitor, a decongestant, a diuretic, a sedating or non-sedating anti-histamine, an inducible nitric oxide synthase inhibitor, an opioid, an analgesic, a Helicobacter pylori inhibitor, a proton pump inhibitor, an isoprostane inhibitor, a vasoactive agent, a α-agonist, an anticholinergic, a mast cell stabilizer, an immunosuppressive agent, a growth factor antagonist or antibody, a dopamine agonist, a radiotherapeutic agent, a heavy metal functioning as a radiopaque agent, a biologic agent, an angiotensin converting enzyme inhibitor, an angiotensin II receptor antagonist, a renin inhibitor, a free radical scavenger, an iron chelator, an antioxidant, a sex hormone, an antipolymerase, an antiviral agent, a photodynamic therapy agent, an antibody targeted therapy agent, a gene therapy agent, or a mixture of two or more thereof.

66. The composition of claim 64, wherein the therapeutic agent has at least one NO group, at least one NO₂ group or at least one NO and NO₂ group, wherein the at least one NO group, at least one NO₂ group or at least one NO and NO₂ group, is linked to the therapeutic agent through an oxygen atom, a nitrogen atom or a sulfur atom.

67. The composition of claim 64, wherein the therapeutic agent is an antiproliferative agent, a steroid, a non-steroidal antiinflammatory agent, an immunosuppressive agent or a mixture of two or more thereof.

68. A method for treating a cardiovascular disease or disorder in a patient in need thereof comprising administering a therapeutically effective amount of the composition of claim 64.

69. The method of claim 68, wherein the cardiovascular disease or disorder is restenosis, coronary artery disease, atherosclerosis, atherogenesis, cerebrovascular disease, angina, ischemic disease, congestive heart failure, pulmonary edema associated with acute myocardial infarction, thrombosis, high or elevated blood pressure in hypertension, platelet aggregation, platelet adhesion, smooth muscle cell proliferation, a vascular or non-vascular complication associated with the use of a medical device, a wound associated with the use of a medical device, vascular or non-vascular wall damage, peripheral vascular disease or neoinitimal hyperplasia following percutaneous transluminal coronary angiograph.

70. The method of claim 69, wherein the cardiovascular disease or disorder is restenosis or atherosclerosis.

71. A method for treating a pathological condition resulting from abnormal cell proliferation, a transplant rejection, an autoimmune, inflammatory, proliferative, hyperproliferative or vascular disease, for reducing scar tissue or for inhibiting wound contraction in a patient in need thereof comprising administering a therapeutically effective amount of the composition of claim 64.

72. The method of claim 71, wherein the pathological condition resulting from abnormal cell proliferation is a cancer, a Karposi's sarcoma, a cholangiocarcinoma, a choriocarcinoma, a neoblastoma, a Wilm's tumor, Hodgkin's disease, a melanoma, multiple myelomas, a chronic lymphocytic leukemia or an acute or chronic granulocytic lymphoma.

73. The method of claim 71, wherein the autoimmune, inflammatory, proliferative, hyperproliferative or vascular diseases is rheumatoid arthritis, restenosis, lupus erythematosus, systemic lupus erythematosus, Hashimotos thyroiditis, myasthenia gravis, diabetes mellitus, uveitis, nephritic syndrome, multiple sclerosis, an inflammatory skin disease, an inflammatory lung disease, an inflammatory bowel disease, an inflammatory disease that affects or causees obstruction of a body passageway, an inflammation of the eye, nose or throat, a fungal infection or a food related allergy.

74. The method of claim 68 or 71, wherein the composition is administered intravenously, orally, bucally, parenterally, by an inhalation spray, by topical application or transdermally.

75. The method of claim 68 or 71, wherein the composition is administered via local administration.

76. The method of claim 75, wherein the local administration of the composition is via a suture, a vascular implant, a stent, a heart valve, a drug pump, a drug delivery catheter, an infusion catheter, a drug delivery guidewire or an implantable medical device.

77. A method for direct delivery of nitric oxide to a targeted site in a patient in need thereof comprising administering the composition of claim 64 directly to the targeted site in the patient.

78. The method of claim 77, wherein the composition provides sustained delivery of nitric oxide to the targeted site in the patient.
